SEQUENCE LISTING (49 sequences)

SEQ ID NO: 1 - DNA, 326 bp, Homo sapiens
tttttcttacacaggcattgtaaaggatgttcatgaagattcaataacagttgcatttgaaaacaagtaagtgtctcgttatattattttaatgatgaggttctttaatattttatgctaattctattcttcattttttaaaaattcaagtccagtttgagtgcttttcaggaatggatcttcatgttactgactgagaagtttctgaacaactcagtattaaactaatggaatgactgtttctgctaatgtcctggaggtcccttattgtatggtattgatccttacgtcttaattcccttgaatgtgaagaaagaaaccagaga

SEQ ID NO: 2 - DNA, 3556 bp, Homo sapiens
agtaagtgtctcgttatataattttaatgatgaggttctttaatattttatgctaattctattcttcattttttaaaaattcaagtccagtttgagtgcttttcaggaatggatcttcatgttactgactgagaagtttctgaacaactcagtattaaactaatggaatgactgtttctgctaatgtcctggaggtcccttattgtatggtattgatccttacgtcttaattcccttgaatgtgaagaaagaaaccagagagtcttgtgtattagtaactggaagttcgcataggagtatttgtaaattttaaaaagatagtaatgaaaagattcatgggtattgctaaaaagttgtatgtgagttctttaacattaaatctaaaactgatggttttagttgtctagtatattttatggaaacagtcatgtttacctattatctgattttttttaatgtttcatatgttcagcaatagcagtagctgatcttttctgttctcttttgctagatgaagaaataaaaacagtcataggcctaggaatattaactgtatgaaagcatcaatagtatagatgttaacattttattggagaacacaagtctcttgactaaatgttttgaatgctaataaaggctattttcagggtagctgttggtaagattgtaaagtacatataaactccttagtcaaagtgtagatgtggctatgatcttaggattttactaaactctgatggatggttaacagttatcatttttttggctcttatataccaagaaaattaataatatatcaaaagcaggctgcaaatctatagagacagaaagtagattagtgattgcttgtgctggggctggtggggagaacatagctaaagagtacaggatttttgtgtgtgtggcaatgaaaatgttgtaaaattgactggtaattattgcacataactgtaaaccttctataaaccattgaattgtacatttaaattgatgaattataatgatatgtggattatgtctgctaaaatgtagtatgtgacccaggcatactgagcaataaatacacacatcttgcttctaaagtttaaaaccaggcctgtgtttcagtgtctagaaaatatatttcataattgaacttagcagattcttttggctagcaacaggacaaagtagtatccttagatgcctgaaacacgtgtagggggacagagataaaaggattaaaagcatctaaccctatttctggctctggactaataatcgcagaccaaaatacaagtttgaatagtagaatacctttccctgaatacataaagccaaaagttttagtatttgtctcttcgctgtttcatatatccatggctatgcaatagacaacagcatcacaccaacagaggaatctttgataacttggagggtatttttttcttacaattatacactttatctacttacttacatcactaggtaaatgctgtttcccataaagggagagttgtctttatttgctactcttttagtttttcctgtagtctataatctaaagtgttaagcaaacactcatttcctctatgcatttaagaatttgtctagtttatacattgatttattagatcacttctttttaattttcaatcagatttctaccataaacttaaaattatttcatctttggaccttccaattatttatctcctgtaccatatctgcatgaaagtttctcttcaagctctttgtcatcctgggagtttgaaatagatcattgattttcccttgtgttcatggagtatgtttgcaatacaacactgggtttgagcacatcaataggatttaataggtctccaccaaagtaatctctttgcccttccttgtgtgtttcctttttggttttctgttagaaacaaaacgaaaacagtgttttgtaaaacaccagattgtatttaccctttctttcctttcaatcaggaagaagctcctgttactgatagcagattgttttaggtgctttgtttttaggtgcttcctaaaccttccataaagcataaaggtgttttcatatttcacattttttaataaggaatttgaatttttatctgtatacacttaaaatcattattttttccttggaaccattgagttatatattattatatgctttaaaatcaaatttagcaatgaatgtataacatctattaggtgtgtataataactcagaggagggttcagttaatttaggccctaatcagatttccacaaattctgacttaatatttgcccgcttatataacagctcttctttaacaaaaacaagtacttttctcaatagaattttactaagaaagctctttagtaaaacatcgacattatacatacaacatatctcagtatctgctgatgaagaacaccaaaaagaacccagatgtgactgctccggaagttgaatcctcagtatttttgcaaagtttgtctttcagtattttatttgtgtgtgtgtgtgtgtgtgtgtgtgtgtgtgtctatatatatatatatttttttttttttaaagacaggatctcactctgtcacctaggctggagtgcagtggcatgatcatggctcactgtaaccttgaactcctgagcttgagctatcctcccacctcagcctcccgagtagctgggactataggcacataccactgcacctaatttttttttttttttaataatttgttgtaaagatcaggtcttaccttgttgcccaggctgctcttgaagtcctggcctgaagcagtgctcccacctcagcctcccaaagctctgggattataggcttgagccaccgcatcctaatattttatatttttatggatataaaaaataatttggtatctttcagagttgtttaatatcattttaaatttaaaaacataggcaacttaaactcctataggctgtctccatcgggtttctgtggtttaggagaccccaccatcccagtgcatgctgataacgtcatactgatcagcatccagctacccacagcaagaattgaccacctcgtgggatctaaaatttaaagggggaaaagtgagttgtgaattgctaatgtgctgatagccccattttgcttgggaattagagggcagtttttgtggtccttggaatgtggttaaaattcttctgcaagtggaagcatatttatattactaacaattactggtactaatattcaaatattgaaggaaatttctgttgtggacttatgtttaaagctcttagaagttgaaattattggaaagaagacttgttttgaaaatcataatgttgctgtattgtgtttagagaaatattccaaacgggagtaggctgctgtgctgcatgcagactttgctgaaatgttactatattgccgttatgtcccactcagcaaaaactgatgattttaaagcttttgcttcttgaaactagaaaaagaggggtcagccttaaccaaaagttgatggcagagtggtcattatttcagttaaacatgaaaagcatgttaaataattgtatgtttgcttatttacagc

SEQ ID NO: 3 - DNA, 9703 bp, Homo sapiens
gtacttggctctagggcaggccccatcttcgcccttccttccctcccttttcttcttggtgtcggcgggaggcaggcccggggccctcttcccgagcaccgcgcctgggtgccagggcacgctcggcgggatgttgttgggagggaaggactggacttggggcctgttggaagcccctctccgactccgagaggccctagcgcctatcgaaatgagagaccagcgaggagagggttctctttcggcgccgagccccgccggggtgagctggggatgggcgagggccggcggcaggtactagagccgggcgggaagggccgaaatcggcgctaagtgacggcgatggcttattccccctttcctaaacatcatctcccagcgggatccgggcctgtcgtgtgggtagttgtggaggagcggggggcgcttcagccgggccgcctcctgcagcgccaagagggcttcaggtctcctttggcttctcttttccggtctagcattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttcctttgaatttgtggtgttgcagtggactgaattgttgaggctttatataggcattcatgggtttactgtgctttttaaagttacaccattgcagatcaactaacacctttcagttttaaaaggaagatttacaaatttgatgtagcagtagtgcgtttgttggtatgtaggtgctgtataaattcatctataaattctcatttccttttgaatgtctataacctctttcaataatatcccaccttactacagtattttggcaatagaaggtgcgtgtggaaggaaggctggaaaatagctattagcagtgtccaacacaattcttaaatgtattgtagaatggcttgaatgtttcagacaggacacgtttggctataggaaaataaacaattgactttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctgcagtcaaaattttaatttcattgattttattgatccataatttcttctggtgagtttgcgtagaatcgttcacggtcctagattagtggttttggtcactagatttctggcactaataactataatacatatacatatatatgtgtgagtaacggctaatggttaggcaagattttgattgacctgtgatataaacttagattggatgccactaaagtttgcttatcacagagggcaagtagcacattatggccttgaagtacttattgttctcttccagcaacttatgatttgctccagtgattttgcttgcacactgactggaatataagaaatgccttctatttttgctattaattccctccttttttgttttgttttgtaacgaagttgtttaacttgaaggtgaatgaagaataggttggttgccccttagttccctgaggagaaatgttaatacttgaacaagtgtgtgtcagacaaattgctgttatgtttatttaattaagtttgatttctaagaaaatctcaaatggtctgcactgatggaagaacagtttctgtaacaaaaaagcttgaaatttttatatgacttataatactgctgtgagttttaaaagtaaagcaaaagtaaactgagttgcttgtccagtgggatggacaggaaagatgtgaaataaaaaccaatgaaaaatgaactgctgtggagaagtgttacatttatggaaaaagaaataggaaccttgttcatcaaattgatagaaaagcttttaaaactaaacaaatcaaacaacttgagtataatggaattcagactttgatttgcctaacataaccaccatatttgcaaggacagctctctatcttctggtgtttattcttaaaaacttaaaagttagatttagcgatcaccagagccactacttttatgcttaggtatttgtttgacttagaaaaaattggtcacgtgtaccactttatagtgccctgcaggtgttaagatatgaaggcactttgacttacacctcataaaatctttacaaagtattttctaaatgaataatgatgaaataaagtctttattctaggtgcatctgccccacataatttgttttctttggactagaagttttgatgtgttgagaatggtaatgaattaactccattttaaatgtagaatgcgtatcactccaatatgaatgccctaatgaatcctaagatttgtaggttttgtgtactagtatgaaaattactaaagatggaaaaatcacatgttggagacataagatacaaacctttttgttttctgaaaatacaacctctgatttctgattccttgttgtaatatggtgtaattatactagattgtaattttgttgttagattatacttttttaagttcagtgtttgaggacagactttcatttggttagtagtattatggcagctagcagctaaatatgataaagtgtacaatcaaaaggatatttttaatgaagatattagtggtctaacatgtcatttcagatacatagctgaaatgtagtaaaatcagttttactacaaataaacttgcataaggtttataaatttataagtttataaatcaacttgggtaaagtgtaaataaacttgcactcgtggtttctctgaagtctcctgagctaactttgcataaaggtgttattctgtacttcgaggaagtgaattattggggtcaaccacattttttttccttcctacagtctgattgccctttttagtttttaggatctttgtggctgcatcatttttccccttttgaactgtgcattttctaaccccatacttaaatattctcataacctccaaattattaattagatgcaacattcagtggtatattactggagtttctgatttctgcccactataggaatgtgcttcctgagaagattgggatcgtgattataataatagttaacaggggatgagtactttctaggtgccaggcactgttctctctgatactttatttgatgtattgttgttattcccattctttaaatgatgcacagagaggttaggtaagtgacttactaccaagtgtcagggccattaagggtcaggattctgaattcctgaaatgatgaaatttagcttgaagaaattggtttgatttcctgcttagttttcaatttcatggtggtctttgattgtattttgtgctataacactgccttagcatcctataactatagttacagtgttatattaccattttttattgttaatacaaagccatcatgaaataattcagtttatgtgccagctttttttgttactaattcttgaaccttggcgctgtacttcttcatgtggatgcctgttaaggaaagataaagttagaaatctttgaccctgctaggaaatttgtctttgttatattgggagctcataaaactgaagtattcaaaagttagaatacatacacacaagaaaaattagtaactaatttaataatgttttctttgcacatgtctctgttgtcttttggtcagagtgaagctaaatgtgtttttcacataatttgtagcctatatgaagtcctggacatgtggtatggttggaaggactgttgatgaggtttattgtctctctttattcttttatgttgttagtgtccccatacaacggggcggggggagtggggacaaaatgataacttgctttatatatgaagccttgggtttgaatcatactgttatcacatttcctatgtccctatcctgtctcctgcaggttttctttcttggttttcttttttggttttctttcttcctttcttcctttcttttcctttctttcctttcttccttttttctttccttttcttttttcctttctttccttcatttctctcctttgctcctttagtttgtttctttgtttctttggtttgtttctttgtttctttggtttgtttctttgtttctttagtttgtttctttgtttctttgtttctttcaacaggtctcactgtcgcccaggttggagtgcagtggtgggatctcagctcactgcaacctccacctctcgggttcaagccagtctcatgcttcagccttccgggtagctgagattacaggcatgtgccaccattcctggctattttttgcatttttagtagagatgaggtttcgccatgttggccaggctgatctcaagttatctgcctgcctcagccctccaaagtgctggaattacaggcatgagacaccgggtccagcttccagcaggttttctttaggagggatattttacaatgctgtaagtttttcctaacgagaattatcatagcactacatgttctgtcttcagtaagtgatacaagcttactgatgatgttgtagttatgttcattggtggtcgggtgtacattgaaactttaacacataatagcctctcctgtgagcagtggttcctgttggtaagatactttactaagggaaggaatgtgaggtgtcgctggggagagtttacccaaataaggatggactttctgtctttgtttcatcagtcctggtaatagaatgtttgaatagatagctctaggcattacatactttcataaatatgattattgtaattacctctttggcccagttgctagtaaattagggaccccttaatgatttatttcctgtttattcaccctgatgaagaacttgtatctcttttaaactgtactttatcgcctttctcaaattccaagattctcatcacattttttttcttcccaaactctaaataaccttttaatattaagtatctttgtggaaacattgttttctttttctatcccaatttttaaagcttttttaaaaaaaagagtgcttttgttgggatgtacattttccaaatgcaaaaacatttatgattctgtgtctcttataaaatatgacactctctacttttctctcatttatttagtgccacctatgtgtgtaatttcattacccacagcagtcttaggaggctggtcgagttccttatttgcagatgaggaatctgaggtccagagatcacttcttggtgagagtctcacagctattaagtattagagccaagattttgaacgtaggtctgattcacagcaaaaccgttaaccactaagtacactgactccagtaagagccctagtcctcacccaatacactttaattcccctgtgcattcattcaaattcattgaatttgctgcctttggaaacctctcaggaacctcctcaacctctcttctctacagacatcagctttgcctgataggtagggatcatagcaaaacacagttttccaaggtggtgataggtggagtgatagtgctctggagatggccaaagaaggaaggtatgagtgtatctgtgggtgggtgagtggtggataaggggaaggacagagccaaaagcgacggctattggaaaaactatgatgagaaacaggaagatggaaccttgttggaattagtgaaagaccttagaattcacaggaggtatttgtccttcacgcgagtgtagaccaaacgtaacctatgagtttcttttattccacttattaaagcagcaaccaaaggtattatataccttctgtattcacttaaaatgactgattttgaaaaagtcatgcaaacatccatttacagataagcctcattaactcaaaggcagtggccctgttgggctctgatgattactcaaaccatttctgacactctgacactacatccggaaatcatccggaaatgcattcagagcctgcaaaagctttttttttttttttttttttttttgaatagctacagcacttgcagatcttcctcctttgagagaatatttgattttaggaaataagcaaatgtagataagtatgattcaactgggcaataacttttaggcaaagagaaaaaaacaaaatatagcaatgtagtaataaggctgatattatagtgcttttatgatgagtctgaaaacagtctccaacatttgtaaatgttttaaatcgggtctgtctactacagtagcccttaagccatatgtagctattgagcacttgacatgttgcaagagtgaattggagactgattttaattttatttaattttaatgtaagtagtcgcacatgacccgtggctactatggtagatggcatagttttagacaagggcagtagtcttgggtagatacttgatttacttagaacatttctttacctagctgtaacaaggttctaatagctgattaaaggacaacatttttagcatgtaatatacagtaaggaactaatgttaattactgccaagatgtataaacattatgaaaccttaaacaaggatgaacacagaagcagtggcccttctttgtaaataaggggtcagttacttcctaataggtgtcttagttttagttaataatctaatagcacccccaaaaagcaaactgttaatttgttattagccatcctgtaaagacaagggagaaatcggggccaggagagtcctctttcccattctccataatttttacgacctatagaaataagagcctaagagtgaccagttcctgcaacctaactcaaaattaggcctctctgttgaagtaatagtgaggcaaacaatcccttgaagcactggggcatgcacccttatgaaatctaataattctaaaatatcattggcagtattaagctagagttctcaaaatatgctgtagaaaatattggtcatttaaatatagctgagtaaaaataagcaagatcaaaatgataagaaaatgttgatttctccccttttgaaccagtaactaactataagggtatatacccatgcttaacttaaaaataattatttagccaccttgggtatagcacaacatatggatgccattatagtccaccttgatcttacaaggaagctttcttttagcgtagctttacttttatttaagcattattgaagaagcttggtatctctgtttaagttgcttctgtatcagtgttttccagggccctgtctcagcttccatagttttccttaagagcagctaattaatgcttcactccatatgctttaatttgatattttgggaagtttcattttttaaaataatctttttatatcataggcctttgaagacatatttgtttacaaagacatctgcattgaaccatttgtttttaaaaacatgtatcgggcatgtcctatataccagaaactatgccatttactgaggacaccgaagttagagaatggtcttaatccgaactagttattgtctcactatccaatatggatatcaaacattcagcgtcatgaggatttactactgtgttttctcccaagagttttataattttagctcttatatttaggtctttgatgcatattcattgatttttttttgtatgcagtatgaggtaaaggttgaaacgtattctttttgcatgtgggtgtccacttgtcccagcttcattttttgaaaacactatactttccccattgaattgtcttggcttctttgttgacaaaggatttattatttctgccacaacccagctgtttctcactgtctaaacctttatgcctgctgccccgtaggcttagtggaatgcctctccatcctccactctcttttgtttagctagtacttccttatcctttaaggggtcagtaggaacctcaccttcttcagtatgttttcctttgtcttatcaacctaccaccatcacccacacacataccttttccctaaggctgggataggaacctcttaggcaatgatctatttgataagtactcactgagttcatactagacactctgcagtatatccgtaaatgaagtaaaacctgacaccaccctcagaaaacttactgttagtgagaaaatgggctataataatagagtggggtaagtttattgtagggataagcataggatgctatgagaatgcagaggacaggaatttaacctagacttctcagttctcccgttgaagttgacatctgaactgaaatctggaagaccagtaagagatagctatgtaaaaagaggggaaggacaataggaaagaaggaatggagagaggcccagaaactacagagtatggcacaagtagtttagcattgttgggtcacaaattctaaggatatgaaagataaagttgaagaagtggtggggctggggaggacagctcctgtagagccttttacagcgggagaaggagtttggctatggtcctgcaggcagggagaaaccactgaaggggagaaacatacagaataaatttgagtaagaagcttaaatctccccaagcccttttaaaataaattaagatataaagcctttgggtttctttatgctttgtcctattcttctaattgtccaaaacaaaacaaaaaccttccttttctgtacctattaaaaggttaattttataaagttacagacagcatgctgattaaagaattcttgataattagctattttgtctgtctttgtgtagattactaacaattgtgtcatcagatttaaaagatctaaacctctggactttatatattttttctacaagtactgtgagattgagaacttaattcaactctagtaactggactttttagtgttgtttgcagcatatgattgttaaagaaagtttagtatatttgtgtgtgcgtatatatatatatatagatagatagatagatttgaggttatatttaatcattgactattggtattctaagattttaaggagagaaaggtaggttacaatatatggctgatatatggttctggaacaattttcttagttttcagtaattctacctaaaatgtacgtccagctttgcactcactaccacattgtaatttccagtatacttgtctatttttcgagatgttgaaatctgtggagaattttttctcatgtttagtgttttagctatgtttatctctagggtatagcacatacaaggtggcaatatagatgtgcttaatgaataaaatgatcttcaaaaactgaccttcatttgaagttctattttattcataaacacataaaacgtttggtatcactgtaaaatttaactaaaaacaaaaactatctttaagctcacaagttaatttaacgttttttcttacacagg

SEQ ID NO: 4 - DNA, 497 bp, Homo sapiens
tgggcctgttggaagcccctctccgactccgagaggccctagcgcctatcgaaatgagagaccagcgaggagaggttctctttcggcgccgagtccccgccggggtgagctgggatgggcgagggccggcggcaggtactagagccgggcgggaagggccgaaatcggcgctaagtgacggcgatggcttattccccctttcctaaacatcatctcccagcgggatccgggcctgtcgtgtgggtagttgtggaggagcggggggcgcttcagccgggccgcctcctgcagcgccaagagggcttcaggtctcctttggcttctcttttccggtctagcattgggacttcggagagctccactgttctgggcgagggctgtgaagaaagagtagtaagaagcggtagtcggcaccaaatcacaatggcaactgatttttagtggcttctctttgtggatttcggaggagattttagatccaaagtttcaggaagacc

SEQ ID NO: 5 - DNA, 303 bp, Homo sapiens
caagagggcttcaggtctcctttggcttctcttttcctggtctagcattgggacttcggagagctccactgttctgggcgagggctgtgaagaaagagtagtaagaagcggtagtcggcaccaaatcacaatggcaactgatttttagtagcttctctttgtggatttcagagaagattttagatccaaaagtttcagaaagaccctaacatggcccagcagtgcattgaagaagttgatcatcgtgaatattcgcgtccccctttttgttaaacggggaaattcaggaatgcacattgcttc

SEQ ID NO: 6 - DNA, 35 bp, Homo sapiens
aggaagagagggtttttttgaaatttttggattta

SEQ ID NO: 7 - DNA, 56 bp, Homo sapiens
cagtaatacgactcactatagggagaaggcttaaaacctattaaaaacccctctcc

SEQ ID NO: 8 - DNA, 35 bp, Homo sapiens
aggaagagagtaagagggttttaggttttttttgg

SEQ ID NO: 9 - DNA, 59 bp, Homo sapiens
cagtaatacgactcactatagggagaaggctaaaacatatacattcctaaatttacccc

SEQ ID NO: 10 - DNA, 40 bp, Homo sapiens
aggaagagagttttttttatataggtatttgtaaaggatg

SEQ ID NO: 11 - DNA, 59 bp, Homo sapiens
cagtaatacgactcactatagggagaaggcttctctaatttctttcttcacattcaaaa

SEQ ID NO: 12 - DNA, 10 bp, Homo sapiens
ggcgatggct

SEQ ID NO: 13 - DNA, 10 bp, Homo sapiens
tgaatattcg

SEQ ID NO: 14 - DNA, 13 bp, Homo sapiens
aagtttccaaaga

SEQ ID NO: 15 - DNA, 10 bp, Homo sapiens
tattattatt

SEQ ID NO: 16 - DNA, 6 bp, Homo sapiens
accaca

SEQ ID NO: 17 - DNA, 7 bp, Homo sapiens
tgtggtg

SEQ ID NO: 18 - DNA, 7 bp, Homo sapiens
tataaat

SEQ ID NO: 19 - DNA, 7 bp, Homo sapiens
tataaat

SEQ ID NO: 20 - DNA, 8 bp, Homo sapiens
aataatat

SEQ ID NO: 21 - DNA, 11 bp, Homo sapiens
aaataaacaat

SEQ ID NO: 22 - DNA, 7 bp, Homo sapiens
cataaat

SEQ ID NO: 23 - DNA, 7 bp, Homo sapiens
tttgttt

SEQ ID NO: 24 - DNA, 7 bp, Homo sapiens
tttgttt

SEQ ID NO: 25 - DNA, 7 bp, Homo sapiens
ttgttta

SEQ ID NO: 26 - DNA, 16 bp, Homo sapiens
tttatttaattaagtt

SEQ ID NO: 27 - DNA, 7 bp, Homo sapiens
aaacaaa

SEQ ID NO: 28 - DNA, 7 bp, Homo sapiens
tataatt

SEQ ID NO: 29 - DNA, 12 bp, Homo sapiens
tttatgctaatt

SEQ ID NO: 30 - DNA, 1520 bp, Homo sapiens
taacatggcccagcagtgcattgaagaagttgatcatcgtgaatattcgcgtccccctttttgtaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttccttgaattgtggtgttgcagtggactgaattgttgaggctttatataggcattcatgggtttactgtgctttttaaagttacaccattgcagatcaactaacacctttcagttttaaaaggaagatttacaaatttgatgtagcagtagtgcgtttgttggtatgtaggtgctgtataaatcatctataaattctcatttccttttgaatgtctataacctcttcaataatatcccaccttactacagtattttggcaatagaaggtgcgtgtggaaggaaggctggaaaatagctattagcagtgtccaacacaattcttaaatgtattgtagaatggcttgaatgtttcagacaggacacgtttggctataggaaaataaacaattgacttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctcagtcaaaattttaatttcattgattttattgatccataatttcttctggtgagtttgcgtagaatcgttcacggtcctagattagtggttttggtcactagatttctggcactaataactataatacatatacatatatatgtgtgagtaacggctaatggttaggcaagatttgattgacctgtgatataaacttagattggatgccactaaagtttgcttatcacagagggcagtagcacattatggccttgaagtacttattgttctcttccagcaacttatgatttgctccagtgattttgcttgcacactgactggaatataagaaatgccttctatttttgctattaattccctccttttttgttttgttttgtaacgaagttgtttaacttgaaggtgaatgaagaataggttggttgccccttagttccctgaggagaatgttaatacttgaacaagtgtgtgtcagacaaattgctgttatgtttatttaattaagtttgatttctaagaaaatctcaaatggtctgcactgatggaagaacagtttctgtaacaaaaaagcttgaaatttttatatgacttataatactgctgtgagttttaaaagtaaagcaaaagtaaactgagttgcttgtccagtgggatggacagg

SEQ ID NO: 31 - DNA, 237 bp, Homo sapiens
tcgagatgttgaaatctgtggagaatttttctcatgtttagtgttttagctatgtttatctctagggtatagcacatacaaggtggcaatatagatgtgcttaatgaataaaatgatcttcaaaactgaccttcatttgagttctattttattcataaacacataaaacgtttggtatcactgtaaaatttaactaaaaacaaaaactatctttaagctcacaagttaatttaacgt

SEQ ID NO: 32 - DNA, 30 bp, Homo sapiens
gctcagctccgtttcggtttcacttccggt

SEQ ID NO: 33 - DNA, 30 bp, Homo sapiens
agccccgcacttccaccaccagctcctcca

SEQ ID NO: 34 - DNA, 17 bp, Homo sapiens
ccgcggaatcccagaga

SEQ ID NO: 35 - DNA, 19 bp, Homo sapiens
cagtggcgtgggaaatcaa

SEQ ID NO: 36 - DNA, 17 bp, Homo sapiens
tgggataaccggatgca

SEQ ID NO: 37 - DNA, 20 bp, Homo sapiens
acaccctgtgccctttaagg

SEQ ID NO: 38 - DNA, 22 bp, Homo sapiens
tcaaagctgggtctgaggaaag

SEQ ID NO: 39 - DNA, 17 bp, Homo sapiens
tcgggatctcaaaatgt

SEQ ID NO: 40 - DNA, 21 bp, Homo sapiens
ccccagaatgagaggatgttg

SEQ ID NO: 41 - DNA, 21 bp, Homo sapiens
gccctagatccaccgctttaa

SEQ ID NO: 42 - DNA, 14 bp, Homo sapiens
tgctggtggaactc

SEQ ID NO: 43 - DNA, 242 bp, Homo sapiens
ctgaagagaagatggaggagctggtggtggaagtgcggggctccaatggcgctttctacaaggtacttggctctagggcaggccccatcttcgcccttccttccctcccttttcttcttggtgtcggcgggaggcaggcccggggccctcttcccgagcaccgcgcctgggtgccagggcacgctcggcgggatgttgttgggagggaaggactggacttggggcctgttggaagcccctct

SEQ ID NO: 44 - DNA, 454 bp, Homo sapiens
acttggggcctgttggaagcccctctccgactccgagaggccctagcgcctatcgaaatgagagaccagcgaggagagggttctctttcggcgccgagccccgccggggtgagctggggatgggcgagggccggcggcaggtactagagccgggcgggaagggccgaaatcggcgctaagtgacggcgatggcttattccccctttcctaaacatcatctcccagcgggatccgggcctgtcgtgtgggtagttgtggaggagcggggggcgcttcagccgggccgcctcctgcagcgccaagagggcttcaggtctcctttggcttctcttttccggtctagcattgggacttcggagagctccactgttctgggcgagggctgtgaagaaagagtagtaagaagcggtagtcggcaccaaatcacaatggcaactgatttttagtggcttct

SEQ ID NO: 45 - DNA, 302 bp, Homo sapiens
caagagggcttcaggtctcctttggcttctcttttccggtctagcattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttc

SEQ ID NO: 46 - DNA, 373 bp, Homo sapiens
aaaagtttcaggaagacctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttcctttgaatttgtggtgttgcagtggactgaattgttgaggctttatataggcattcatgggtttactgtgcttttt

SEQ ID NO: 47 - DNA, 261 bp, Homo sapiens
aaagttacaccattgcagatcaactaacacctttcagttttaaaaggaagatttacaaatttgatgtagcagtagtgcgtttgttggtatgtaggtgctgtataaattcatctataaattctcatttccttttgaatgtctataacctctttcaataatatcccaccttactacagtattttggcaatagaaggtgcgtgtggaaggaaggctggaaaatagctattagcagtgtccaacacaattcttaaatgtattgta

SEQ ID NO: 48 - DNA, 360 bp, Homo sapiens
gaatggcttgaatgtttcagacaggacacgtttggctataggaaaataaacaattgactttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctgcagtcaaaattttaatttcattgattttattgatccataatttcttctggtgagtttgcgtagaatcgttcacggtcctagattagtggttttggtcactagatttctggc

SEQ ID NO: 49 - DNA, 327 bp, Homo sapiens
tttttcttacacaggcatttgtaaaggatgttcatgaagattcaataacagttgcatttgaaaacaagtaagtgtctcgttatataattttaatgatgaggttctttaatattttatgctaattctattcttcattttttaaaaattcaagtccagtttgagtgcttttcaggaatggatcttcatgttactgactgagaagtttctgaacaactcagtattaaactaatggaatgactgtttctgctaatgtcctggaggtcccttattgtatggtattgatccttacgtcttaattcccttgaatgtgaagaaagaaaccagaga

(19) United States
(12) Patent Application Publication
(10) Pub. No.: US 2014/0212873 A1
(43) Pub. Date: Jul. 31, 2014

(54) TREATMENT AND DIAGNOSIS OF EPIGENETIC DISORDERS AND CONDITIONS

(71) Applicant: David Eugeny Godler, Hughesdale (AU)

(72) Inventor: David Eugeny Godler, Hughesdale (AU)

(73) Assignees: LA TROBE UNIVERSITY, Bundoora, VICTORIA (AU); MURDOCH CHILDRENS RESEARCH INSTITUTE, Parkville, VICTORIA (AU)

(21) Appl. No.: 14/128,319

(22) PCT Filed: Jun. 22, 2012
(86) PCT No.: PCT/AU2012/000731

§ 371 Date: Apr. 3, 2014

(30) Foreign Application Priority Data
Jun. 24, 2011 (AU) 2011902500

(51) Int. Cl. C12Q 1/68; G06F 19/22

(52) U.S. Cl. CPC C12Q 1/6881; G06F 19/22
USPC 435/6.11; 702/19

(57) ABSTRACT

The present disclosure relates generally to the field of epigenetics and in particular epigenetic profiles associated with a pathological condition. The present specification teaches screening of individuals and populations for epigenetic profiles associated with a pathological condition. Epigenetic profiles are disclosed from the following sites in the FMR1 gene: FREE3, intron 2, an intron, intron/exon boundary and/or splicing region downstream of intron 2, and a site within the FREE2 portion of intron 1 in combination with a FM. Epigenetic profiles are also disclosed from a region in the FMR genetic locus selected from an intron, intron/exon boundary, a splicing region or an intragenic region in combination with an expansion mutation. Kits and diagnostic assays are also taught herein as are computer programs to monitor changes in epigenetic patterns and profiles. Further enabled herein is a method for screening for agents which can reduce or mask the adverse effects of epigenetic modification and the use of these agents in therapy and prophylaxis.

### FILING DATA

This application is associated with and claims priority from Australian Provisional Patent Application No. 2011902500, filed on 24 Jun., 2011, entitled “Treatment and diagnosis of epigenetic disorders and conditions”, the entire contents of which, are incorporated herein by reference.

### FIELD

The present disclosure relates generally to the field of epigenetics and in particular epigenetic profiles associated with a pathological condition. The present specification teaches screening of individuals and populations for epigenetic profiles associated with a pathological condition. Kits and diagnostic assays are also taught herein as are computer programs to monitor changes in epigenetic patterns and profiles. Further enabled herein are methods for screening for agents which reduce or mask the adverse effects of epigenetic modification and the use of these agents in therapy and prophylaxis.

### BACKGROUND

Bibliographic details of the publications referred to in this specification are also collected at the end of the description.

Reference to any prior art in this specification is not, and should not be taken as, an acknowledgment or any form of suggestion that this prior art forms part of the common general knowledge in any country.

It is apparent that DNA methylation and other epigenetic modifications play a role in the regulation of gene expression in higher organisms. The importance of epigenetic modification has been highlighted by its involvement in several human diseases. Methylation, for example, of cytosine at the 5′ position is the only known methylation modification of genomic DNA. In particular, methylation of CpG islands within regulatory regions of the genome appears to be highly tissue specific. Methylation of cytosines distal to the islands is also important. These regions are called “shores” or “island shores” (Irizarry et al. (2009) Nature Genetics 41(2):178-186). Epigenetic modifications include histone modification, changes in acetylation, methylation, obiquitylation, phosphorylation, sumoylation, activation or deactivation, chromatin altered transcription factor levels and the like.

Another genetic condition which can affect gene expression arises from expansion or increase in the number of repeats in a specific tandem repeat array. Such nucleotide expansion can result in repeat expansion disease conditions. A critical threshold of repeat expansion determines the level of pathologenicity (Orr and Zoghbi (2007) Ann Rev Neurosci 30:575-621). Many diseases arise from expansion of a repeat located in an open reading frame resulting in a protein with a long polyQ² tract that is toxic to neurons (Orr and Zoghbi, 2007 supra). Other expansion disease conditions such as Fragile X syndrome (FXS), Fragile XE mental retardation (FRAXE), Fragile type, folic acid type, rare 12 (FRA12A), mental retardation (MR), Friedrich's ataxia (FRDA) and myotonic dystrophy (DM), arise from transcription of the repeats which are not translated.

A particular type of expansion disorder is referred to as a trinucleotide repeat disorder (also known as trinucleotide repeat expansion disorder, triplet repeat expansion disorder and codon reiteration disorder) and results from trinucleotide repeats in certain genetic loci. An example occurs in the Fragile X Mental Retardation genetic locus (“FMR genetic locus”).

The FMR genetic locus includes the FMR1 gene which is composed of 17 exons, spanning 38 Kb, and encodes Fragile X Mental Retardation Protein (FMRP), essential for normal neurodevelopment (Verkerk et al. (1991) Cell 65(5): 905-914; Terracciano et al, (2005) Am J Med Genet C Semin Med Genet 137C(1):32-37). A CGG repeat segment is located within the 5′ untranslated region (UTR) of the gene. Its normal range is <40 repeats. When expanded, these repeats have been implicated in a number of pathologies, including the Fragile X syndrome (FXS), Fragile X-associated Tremor Ataxia Syndrome (FXTAS) and Fragile X-associated primary ovarian insufficiency (FXPOI; formerly referred to as Premature Ovarian Failure [POF]). FXS is neurodevelopmental in nature with a frequency of 1/1400 males and 1/8000 females, associated with a Fragile site at the Xq27.3 locus (Jin and Warren (2000) Hum. Mol. Genet 9(6):901-908).

This syndrome is caused by a CGG expansion to “full mutation” (FM) which comprises >200 repeats, leading to a gross deficit of FMRP and subsequent synaptic abnormalities (Pieretti et al. (1991) Cell 66(4):817-822; Irwin et al. (2000) Cereb Cortex 10(10):1038-1044). The FXS clinical phenotype ranges from learning disabilities to severe mental retardation and can be accompanied by a variety of physical and behavioral characteristics. FXTAS is prevalent in ˜30% of premutation individuals (PM), comprising −55 to 199 repeats (Nolin et al. (2003) Am J Hum Genet 72(2):454-464) and is a progressive neurodegenerative late-onset disorder with a frequency of 1/3000 males in the general population (Jacquemont et al. (2004) Am J Ment Retard 109(2):154-164), manifesting as tremor, imbalance and distinct MRI and histological changes (Hagerman et al. (2001) Neurology 57(1):127-130; Jacquemont et al. (2005) J Med Genet 42(2):e14; Loesch et al. (2005) Clin Genet 67(5):412-417). It is often associated with ‘toxicity’ of elevated FMR1 mRNA, which has been linked to the intranuclear inclusions and cell death observed during neurodegeneration (Jin et al. (2003) Neuron 39(5):739-747.

FXTAS can occur in females carrying PM, but with much lower frequency as can be expected from X-linked inheritance. The intermediate or Gray Zone (GZ) alleles comprising 41 to 54 repeats (Bodega et al. (2006) Hum Reprod 21(4):952-957) are the most common form of the expansion, 1 in 30 males and 1 in 15 females. As with PM alleles, increased levels of FMR1 mRNA have been reported in the GZ individuals, proportional to the size of CGG expansion (Kenneson et al. (2001) Hum Mol Genet 1004):14491454; Mitchell et al. (2005) Clin Genet 67(1):38-46; Loesch et al. (2007) J Med Genet 44(3):200-204). Female carriers of both PM and GZ allelic types have an increased risk of developing POF (Allingham-Hawkins et al. (1999) Am J Med Genet 83(4):322-325; Sullivan et al. (2005) Hum Reprod 20(2):402-412) which has incidence of approximately 1% in the general population, and often unknown etiology (Coulam (1982) Fertil Steril 38(6):645-655).

Expansion related abnormalities in FMR1 are involved in pathologies with a wide spectrum of patho-mechanisms all pointing to involvement of multiple factors at the Xq27.3 locus in addition to FMR1. A number of antisense transcripts have been described embedded within the FMR1 sequence, ASFMR1 (Ladd et al. (2007) Hum Mol Genet 16(24):3174-3187) and FMR4 (Khalil et al. (2008) PLoS ONE 3(1):e1486). The ASFMR1 and FMR4 transcripts have been suggested to share the bi-directional promoter with FMR1, which is heavily regulated by the state of the surrounding chromatin environment (Pietrobono et al. (2002) Nucleic Acids Res 30(14):3278-3285; Chiurazzi et al. (1998) Hum Mol Genet 7(1):109113).

Transcription of ASFMR1 is also regulated by another promoter located in the exon 2 of FMR1, with the resulting transcript spanning the CGG repeat in the antisense direction (Ladd et al. (2007) supra), and an open reading frame (ORF) with the CGG encoding a polyproline peptide (Ladd et al. (2007) supra). FMR4, however, is a long non-coding RNA, involved in regulation of apoptosis (Khalil et al. (2008) supra).

The length of the CGG repeat has been reported to effect transcription of all three genes FMR1, FMR4 and ASFMR1 (Ladd et al. (2007) supra; Khalil et al. (2008) supra). However, although it is well documented that FMR1 transcription is promoter methylation dependent, linked to the CGG expansion size, the relationship between FMR4- and ASFMR1 transcription and methylation remains elusive.

The FM alleles occur with approximate frequencies of 1 in 4000 males and 1 in 8000 females (Loesch et al. (2003) Am J Med Genet A 118A(2):127-134; Turner et al. (1996) Am J Med Genet 64(1):196-197). However, only 25% of all FM human females can be classified as intellectually disabled (Boyle and Kaufmann (2010) Am J Med Genet C Semin Med Genet 154C(4):469-476). Between 30% and 50% of FM human females carry the abnormal allele predominantly on the inactive X chromosome and have a normal IQ (de Vries et al. (1996) Am J Hum Genet 58(5):1025-1032; Taylor et al. (1994) Jama 271(7):507-514). Up to 25% of all FM carriers are either unmethylated/partially methylated FM carriers or CGG repeat mosaics. These individuals may have a milder FXS phenotype if the smaller PM allele is predominant or if the FM allele is unmethylated or partially methylated (Devys et al. (1992) Am J Med Genet 83(4):208-216; de Vries et al. (1996) supra; Taylor et al. (1994) supra; Hagerman et al. (1994) Am J Med Genet 51(4):298-308). Thus, it is the methylation state of the FMR1 promoter and FMRP expression that is more reflective of the FMR1 function and clinical outcome than just the CGG size.

Methylation sensitive Southern blot analysis combined with PCR is the current ‘gold standard’ for molecular diagnosis of the Fragile X Syndrome (FXS), providing information on the size of the CGG expansion, as well as the methylation status of the FMR1 promoter (Pieretti et al. (1991) supra). For females carrying expanded alleles, the FMR1 activation ratio, which is the proportion of the normal size allele on the active X chromosome, can be also determined using this approach (de Vries et al. (1996) supra). The methylation status of the expanded alleles and the activation ratio in females has been previously shown to be correlated with the levels of FMRP and cognitive status assessed using the Wechsler Adult Intelligence Scale (de Vries et al. (1996) supra; Kaufmann et al. (1999) Am J Med Genet 83(4):286-295).

High-throughput tests for Fragile X syndrome have also been developed, which are either based on expansion size determination using PCR (Filipovic-Sadic et al. (2010) Clin Chem 56:399-408; Hantash et al. (2010) Genet Med 12(3):162-173; Lyon et al. (2010) J Mol Diagn 12(4):505-511), or analysis of methylation using PCR and MLPA within close proximity to the CGG expansion within the FMR1 exon 1 (Hornstra et al. (1993) Hum Mol Genet 2(10):1659-1665; Boyd et al. (2006) Anal Biochem 354(2):266-273; Nygren et al. (2008) J Mol Diagn 10(6):496-501; Zhou et al. (2006) Clin Chem 52(8):1492-1500; Dahl et al. (2007) Clin Chem 53(4):790-793; Dahl and Guldberg (2007) Clin Chem 53(11):1877-1878; Dalh and Guldberg (2007) Nucleic Acids Res 35(21):e144; Weisenberger et al. (2005) Nucleic Acids Res 33(21):6823-6836; Weinhausel and Haas (2001) Hum Genet 108(6):450-458; Coffee et al. (2009) Am J Hum Genet 85(4):503-514; Stoger et al. (1997) Hum Mol Genet 6(11):1791-1801; Rosales-Reynoso et al. (2007) Genet Test 11(2):153-159; Zhou et al. (2004) J Med Genet 41(4):e45). The main limitation of these methylation based tests is that the assays do not reliably detect the type and severity of FXS phenotype, particularly in FM human females (Coffee et al. (2009) supra).

Furthermore, there are issues associated with the CGG sizing tests, as these do not provide information on the methylation status of the FMR1 promoter and, if considered in the context of newborn screening, they can identify PM or GZ mutations that are potential markers of late onset diseases (Hagerman et al. (2001) supra; Greco et al. (2002) Brain 125(Pt 8):1760-1771; Allingham-Hawkins et al. (1999) supra; Sullivan et al. (2005) supra; Loesch et al. (2011) Genet Med: [in press]). Therefore, there is justified concern for use of CGG sizing for presymptomatic screening in children or newborns due to widely held ethical views against performing predictive testing for non-preventable adult-onset disorders in persons under 18 years (Anido et al. (2007) I Genet Couns. 16(1):97-104; Bailey et al. (2009) J Pediatr Psychol 34(6):648-661; Kemper and Bailey (2009) Acad Pediatr 9(2):114-117; Coffee (2010) Genet Med 12(7):411-412; Godler et al. (2010) Genet Med 12(9):595).

Despite the availability of a range of methylation and nucleotide expansion assays (see, for example, Rein et al. (1998) Nucleic Acids Res. 26:2255 in relation to methylation assays), selection of regions to amplify and screen is an important aspect of determining an epigenetic profile characteristic of a disease condition. There is a need to identify crucial regions which are associated with epigenetic change linked to a pathological condition to assay and/or therapeutically target.

### SUMMARY

Throughout this specification, unless the context requires otherwise, the word “comprise” or variations such as “comprises” or “comprising”, will be understood to imply the inclusion of a stated element or integer or method step or group of elements or integers or method steps but not the exclusion of any element or integer or method step or group of elements or integers or method steps.

Nucleotide and amino acid sequences are referred to by a sequence identifier number (SEQ IN NO:). The SEQ ID NOs: correspond numerically to the sequence identifiers <400>1 (SEQ ID NO:1), <400>2 (SEQ ID NO:2), etc. A summary of sequence identifiers is given in Table 1.

Aspects enabled herein are predicated in part on the determination of an association between epigenetic modification of intronic regions including intron/exon boundaries and splicing regions within a genetic locus and a pathological condition. In an embodiment, the epigenetic modification occurs in:

(i) two or more of (a) an intron; (b) an intron/exon boundary; and/or (c) a splicing region;

(ii) approximately one seventh or greater of an intron including an intron/exon boundary and/or a splicing region; and/or

(iii) an intragenic site in combination with an expansion mutation;

within a genetic locus. An “intragenic site” extends to an intron including its intron/exon boundaries. An expansion mutation includes a CGG expansion mutation.

Conditions contemplated herein include pathoneurological conditions such as pathoneurodevelopmental and pathoneurodegenerative conditions as well as non-neurological conditions. Conditions and disorders contemplated herein include polyglutamine (polyQ) diseases such as Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA1), spinocerebella ataxia Type 17 (SCA17) and non-polyQ diseases such as Fragile X syndrome (FXS), Fragile X-associated tremor or ataxia (FXTAS), Fragile XE mental retardation (FRAXE), myotonic dystrophy (DM), spinocerebella ataxia (SCA8) and spinocerebella ataxias Type 12 (SCA12). Other conditions contemplated herein include premutation related disorders including but not limited to Fragile X-associated primary ovary insufficiency (FXPOI), Friedrich's ataxia (FRDA), Fragile type, folic acid type, rare 12 (FRA12A), autism (including co-morbid autism), mental retardation (MR), Klinefelter's syndrome, RNA toxicity disease, Turner's syndrome, a modified X-chromosome and cognitive impairment. Further contemplated herein are learning and behavioral problems.

It is proposed herein that epigenetic changes in an intron, intron/exon boundary and/or splicing region within particular genetic locus are associated with the development, progression and severity of a range of pathological conditions such as but not limited to those listed above. In particular, the epigenetic modification occurs in:

(i) two or more of (a) an intron; (b) an intron/exon boundary; and/or (c) a splicing region;

(ii) approximately one 7^{th} (seventh) or greater of an intron including an intron/exon boundary and/or a splicing region; and/or

(iii) an intragenic site in combination with an expansion mutation;

within a genetic locus.

By “epigenetic modification” includes epigenetic modifications such as methylation including hypermethylation, histone modification, changes in acetylation, obiquitylation, phosphorylation, sumoylation, activation or deactivation, chromatin altered transcription factor levels and the like. The epigenetic modification extends to an increase or decrease in epigenetic change relative to a normal control. In a particular embodiment, epigenetic modification includes the methylation state of CpG and CpNpG sites within an intron of a genetic locus. In one embodiment, the genetic locus is the FMR genetic locus which includes FMR1, FMR4 and ASFMR1 genes. In an embodiment, the epigenetic modification occurs in:

(a) the FMR genetic locus selected from:
(i) two or more of (a) an intron; (b) an intron/exon boundary; and/or (c) a splicing region; and/or
(ii) approximately one 7^{th} (seventh) or greater of an intron including an intron/exon boundary and/or a splicing region; and/or

(b) the FMR1 gene selected from:
(i) an intron including an intron/exon boundary and/or splicing region downstream of intron 1; and/or
(iii) in a site in Fragile X-related epigenetic element 2 (FREE2) region of intron 1 including its intron/exon boundary and in combination with an expansion mutation.

Furthermore, the epigenetic profile is also informative as to the spectrum of disease conditions associated with a particular genetic locus, such as in relation to the FMR genetic locus, whether the subject is normal or has a PM, GZ or FM pathology and/or whether the epigenetic change and/or CGG expansion is heterozygous or homozygous at the FMR allele. For example, hypermethylation of one or more sites within the FREE2 region of intron 1 in an FM subject is associated with cognitive impairment. Reference to the “FREE2 region of intron 1” means portions of FREE2 (A), FREE2 (B) and FREE2 (C) located within intron 1 of the FMR1 gene which includes an intron/exon boundary.

Accordingly, an aspect enabled herein is a method for identifying an epigenetic profile in the genome of a cell indicative of a pathological condition, the method comprising screening for a change relative to, a control in the extent of epigenetic modification within (i) two or more of (a) an intron; (b) an intron/exon boundary; and/or (c) a splicing region; and/or (ii) approximately one seventh or greater of an intron including an intron/exon boundary and/or a splicing region; and/or (c) an intragenic region of a genetic locus in combination with an expansion mutation; wherein the extent of epigenetic change relative to a control is indicative of the presence or severity of the pathological condition or a propensity to develop same.

In another embodiment, the present disclosure teaches a method for identifying an epigenetic profile in a genome of a cell indicative of a pathological condition selected from Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA1), spinocerebella ataxia Type 17 (SCA17), Fragile X-associated tremor or ataxia (FXTAS), Fragile XE mental retardation (FRAXE), myotonic dystrophy (DM), spinocerebella ataxia (SCA8) and spinocerebella ataxias Type 12 (SCA12), premutation-related disorders including FXPOI, Friedrich's ataxia (FRDA), Fragile type, folic acid type, rare 12 (FRA12A), autism (including co-morbid autism), mental retardation, Klinefelter's syndrome, RNA toxicity disease, Turner's syndrome, a modified X-chromosome and cognitive impairment, the method comprising screening for a change relative to a control in the extent of epigenetic modification within (i) two or more of (a) an intron; (b) an intron/exon boundary; and/or (c) a splicing region; and/or (ii) approximately one 7^{th} (seventh) or greater of an intron including an intron/exon boundary and/or a splicing region; and/or (iii) an intragenic region in combination with an expansion mutation; of a genetic locus associated with the pathological condition wherein the extent of epigenetic change is indicative of the presence or severity of the pathological condition or a propensity to develop same. This method further extends to learning and behavioral problems.

In an embodiment, the epigenetic change is in the FMR genetic locus (which includes the FMR1, FMR4 and ASFMR1 genes) and in particular within an intron, intron/exon boundary and/or splicing region downstream of intron 1 of the FMR1 gene, two or more introns, intron/exon boundaries and/or splicing regions of the FMR1 gene, approximately one seventh or greater of one or more introns within a gene of the FMR genetic locus including the FMR1 gene and/or within one or more sites in the FREE2 portion of intron 1 of the FMR1 gene in combination with an expansion mutation. In an embodiment, the expansion mutation is a CGG expansion mutation and in particular an FM (i.e. >200 repeats).

A further aspect taught herein is a method for identifying an epigenetic profile in the genome of a cell indicative of a pathological condition associated with the FMR genetic locus, the method comprising extracting genomic DNA from the cell and subjecting the DNA to an amplification reaction using primers selective for (i) two or more of (a) an intron; (b) an intron/exon boundary; and/or (c) a splicing region; and/or (ii) approximately one seventh or greater of an intron including an intron/exon boundary and/or a splicing region; and/or (iii) a site within the FREE2 portion of intron 1 of the FMR1 gene in combination with a CGG FM expansion mutation within the FMR genetic locus, subjecting the amplified DNA to an epigenetic assay to determine the extent of epigenetic modification of the DNA wherein a change in the extent of epigenetic modification is indicative of the presence or severity of the pathological condition or propensity to develop same.

Reference to the “FMR genetic locus” includes the FMR1, FMR4 and ASFMR1 genes and corresponds to Xq27.3. The term “FMR locus” means the “FMR genetic locus”. In an embodiment, aspects taught herein determine that the intronic region downstream of intron 1 comprises Fragile X-related Epigenetic Element 3(I) as defined by SEQ ID NO:1 or a homolog thereof or a portion or fragment thereof defined by having at least 80% nucleotide sequence identity to SEQ ID NO:1 or which hybridizes to SEQ ID NO:1 or its complementary form under medium stringency conditions; or is intron 2 as defined by SEQ ID NO:2 or a homolog thereof or a portion or fragment thereof defined by having at least 80% nucleotide sequence identity to SEQ ID NO:2 or which hybridizes to SEQ ID NO:2 or its complementary form under medium stringency conditions; or is a site within the FREE2 portion of intron 1 of the FMR1 gene. The nucleotide sequence of intron 1 of the FMR1 gene is set forth in SEQ ID NO:3 and extends to a homolog thereof or a portion or fragment thereof defined by having at least 80% nucleotide sequence identity to SEQ ID NO:3. Further contemplated herein is the amplification of all or part of an expansion mutation and/or and detecting extent of epigenetic change therein in combination with an epigenetic change in (i) two or more of (a) an intron; (b) an intron/exon boundary; and/or (c) a splicing region; and/or (ii) approximately one 7^{th} (seventh) or greater of an intron including an intron/exon boundary and/or a splicing region; and/or (iii) the portion of FREE2 in intron 1 of the FMR1 gene in the FMR genetic locus. The extent of epigenetic change in two or more introns, intron/exon boundaries and/or splicing regions or in one seventh or greater of an intron or within the FREE2 portion of intron 1 of the FMR1 gene within the FMR genetic locus may be determined alone in the case of (i) or (ii) or in combination in the case of (i), (ii) or (iii) with extent of (CGG)ₙ expansion and/or any other epigenetic change therein. The determination of epigenetic change may also be conducted in combination with an assay as contemplated by International Patent Application No. PCT/AU2010/000169 filed on 17 Feb. 2010, the contents of which are incorporated herein by reference in their entirety. In a particular embodiment, the epigenetic modification is methylation.

Another aspect of the present disclosure contemplates a method for identifying a pathological condition in a mammalian subject including a human, the method comprising screening for a change relative to a control in the extent of change in methylation or other epigenetic modification within a region selected from:

(i) Fragile X-related Epigenetic Element 3 (I) in FMR1 comprising the nucleotide sequence set forth in SEQ ID NO:1 or a homolog thereof or portion or fragment thereof defined by having at least 80% nucleotide sequence identity to SEQ ID NO:1 or which hybridizes to SEQ ID NO:1 or its complementary form under medium stringency conditions;

(ii) intron 2 of FMR1 comprising the nucleotide sequence set forth in SEQ ID NO:2 or a homolog thereof or a portion or fragment thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of FMR1 or a homolog thereof or a portion or fragment thereof;

(iv) two or more of (a) an intron; (b) an intron/exon boundary; and/or (c) a splicing region within the FMR genetic locus;

(v) approximately one seventh or greater of an intron including an intron/exon boundary and/or a splicing region within the FMR genetic locus; and

(vi) a site with the FREE2 portion of intron 1;

wherein a change in epigenetic modification relative to a control is indicative of the presence or severity of the pathological condition or a propensity to develop same.

Hence, in relation to detecting epigenetic changes in (i) two or more of (a) an intron; (b) an intron/exon boundary; and/or (c) a splicing region; and/or (ii) approximately one 7^{th} (seventh) or greater of an intron including an intron/exon boundary and/or a splicing region or FREE 3 (I) within intron 2; and/or (iii) within a site in the FREE2 portion of intron 1 of the FMR1 gene in combination with an FM, enabled herein is the diagnosis, monitoring or analyzing of a spectrum of neurodegenerative or neurodevelopmental pathologies such as Fragile X-related conditions including FXS, FXTAS, FRA12A, FXPOI, autism, mental retardation, Klinefelter's syndrome, RNA toxicity disease, Turner's syndrome, a modified X-chromosome and cognitive impairment. Other disorders are also encompassed herein including certain tri-nucleotide disorders.

A “modified” X-chromosome includes an inactivated X-chromosome or an X-chromosome having a skewed X-inactivation, or inversion, insertion, deletion, duplication or is a hybrid.

The epigenetic profile is determined in the genome of a cell of a subject. Any cell may be tested such as a cell from a post-natal or pre-natal human or embryo. More particularly, the cell is, a cultured or uncultured chorionic villi sample (CVS) cell, a lymphoblast cell, a blood cell, a buccal cell, an amniocyte or an EBV transformed lymphoblast cell line. A blood test is also contemplated such as when screening for an epigenetic change in the FREE2 portion of intron 1 in the FMR1 gene in combination with an expansion mutation (e.g. an FM) in a subject. Such an epigenetic change is proposed herein to be indicative of potential cognitive impairment.

In a particular embodiment, the epigenetic modification is methylation of CpG and/or CpNpG sites. Methylation is determined by a range of assays including bisulfite MALDI-TOF methylation assay. In an alternative embodiment, methylation is determined by use of methylation sensitive PCR, methylation specific melting curve analysis (MS-MCA) or high resolution melting (MS-HRM); quantification of methylation by MALDI-TOF MS; methylation specific MLPA; methylated-DNA precipitation and methylation-sensitive restriction enzymes (COMPARE-MS); or methylation sensitive oligonucleotide microarray; or antibodies. Other methods include NEXT generation (GEN) and DEEP sequencing or pyrosequencing. However, any assay of methylation status may be employed.

Further taught herein is a method for screening for an agent which modulates epigenetic change of (i) two or more of (a) an intron; (b) an intron/exon boundary; and/or (c) a splicing region; and/or (ii) approximately one 7^{th} seventh or greater of an intron including an intron/exon boundary and/or a splicing region; and/or (iii) an intragenic region within a genetic locus in combination with an expansion mutation, the method comprising screening for a change relative to a control in the extent of epigenetic modification within the intron in the presence or absence of an agent to be tested, wherein an agent is selected if it induces a change in the epigenetic modification.

A method is also provided for screening for an agent which modulates epigenetic change of an FMR genetic locus in a mammalian cell including a human cell, the method comprising screening for a change relative to a control in the extent of epigenetic modification within a region selected from:

(i) Fragile X-related Epigenetic Element 3 (I) (I)] in FMR1 comprising the nucleotide sequence set forth in SEQ ID NO:1 or a homolog thereof or a portion or fragment thereof defined by having at least 80% nucleotide sequence identity to SEQ ID NO:1 or which hybridizes to SEQ ID NO:1 or its complementary form under medium stringency conditions;

(ii) intron 2 of FMR1 comprising the nucleotide sequence set forth in SEQ ID NO:2 or a homolog thereof or a portion or fragment thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of FMR1 or a homolog thereof or a portion or fragment thereof;

(iv) two or more of (a) an intron; (b) an intron/exon boundary; and/or (c) a splicing region within the FMR genetic locus;

(v) approximately one seventh or greater of an intron including an intron/exon boundary and/or a splicing region within the FMR genetic locus; and

(vi) a site within the FREE2 portion of the intron 1;

in the presence or absence of an agent to be tested wherein the agent is selected if (i) it induces a change in extent of epigenetic modification and/or (ii) causes an improvement in disease phenotype based on the type and degree of epigenetic modification.

As indicated above, in a particular embodiment, the epigenetic modification is methylation. De-methylation as well as pro-methylation, agents are contemplated herein.

Furthermore, also as indicated above, “FREE2” means the portion of FREE(A), FREE(B) and FREE2 (C) located in intron 1 of the FMR1 gene.

Further enabled herein is a method for monitoring the treatment of a disease condition such as Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA7), spinocerebella ataxia Type 17 (SCA17), Fragile X-associated tremor or ataxia (FXTAS), Fragile XE mental retardation (FRAXE), Fragile type, folic acid type, rare 12 (FRA12A), Friedrich's ataxia (FRDA), FXPOI and other premutation related conditions, myotonic dystrophy (DM), spinocerebella ataxia (SCA8) and spinocerebella ataxias Type 12 (SCA12), autism, mental retardation (MR), Klinefelter's syndrome, RNA toxicity disease, Turner's syndrome, a modified X-chromosome and cognitive impairment, the method comprising screening for a change relative to the control in the extent of epigenetic modification within an intron of a genetic locus, wherein the extent of epigenetic change is indicative of the presence or severity of the pathological condition, wherein the treatment modulates the extent of epigenetic change of (i) two or more of (a) an intron; (b) an intron/exon boundary; and/or (c) a splicing region; and/or (ii) approximately one 7^{th} seventh or greater of an intron including an intron/exon boundary and/or a splicing region; and/or (iii) an intragenic region in combination with an expansion mutation within a genetic locus, the method comprising monitoring for a change relative to a control in a pre- and post-treatment sample in the extent of epigenetic modification within the intron, wherein a change in extent of epigenetic modification after or during treatment is indicative of effective treatment. This method also applies to learning and behavioral problems.

By “monitoring” in this context includes diagnosis of disease, monitoring progress of the disease before or after treatment, prognosis of the disease development or remission as well as the pharmacoresponsiveness or pharmacosensitivity of a subject or agent.

Also provided herein for the use of an epigenetic profile within (i) two or more of (a) an intron; (b) an intron/exon boundary; and/or (c) a splicing region; and/or (ii) approximately one 7^{th} seventh or greater of an intron including an intron/exon boundary and/or a splicing region; and/or (iii) an intragenic region of a genetic locus in combination with an expansion mutation in a cell in the manufacture of an assay to identify an epigenetic profile of gene associated with a pathological condition.

An embodiment herein is directed to the use of an epigenetic profile within the FMR genetic locus in a mammalian cell including a human cell, the epigenetic profile including methylation of CpG and/or CpNpG sites located in a region selected from:

(i) Fragile X-related Epigenetic Element 3 (I) [FREE3 (I)] in FMR1 comprising the nucleotide sequence set forth in SEQ ID NO:1 or a homolog thereof or portion or fragment thereof defined by having at least 80% nucleotide sequence identity to SEQ ID NO:1 or which hybridizes to SEQ ID NO:1 or its complementary form under medium stringency conditions;

(ii) intron 2 comprising the nucleotide sequence set forth in SEQ ID NO:2 or a homolog thereof or portion or fragment thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of FMR1 or a homolog thereof or portion or fragment thereof;

(iv) two or more of (a) an intron; (b) an intron/exon boundary; and/or (c) a splicing region within the FMR genetic locus;

(v) approximately one seventh or greater of an intron including an intron/exon boundary and/or a splicing region within the FMR genetic locus; and

(vi) a site within the FREE2 portion of intron 1;

in the manufacture of an assay to identify an epigenetic profile of an FMR locus-associated pathological condition.

The assay taught herein may also be used alone or in combination with assays to detect extent of a nucleotide expansion such as a (CGG)ₙ expansion, such as suing PCR and Southern blot assays. This is particularly useful in determining homozygosity, heterozygosity and mosaicism of a disease or condition. The assay herein is also useful in population studies such as epidemiological studies as well as studies based on ethnic populations. Accordingly, another aspect enabled herein provides a method of identifying epigenetic profile in populations of subjects indicative of a pathological condition associated with epigenetic modifications or changes in an intron, intron/exon boundary and/or splicing region, the method comprising screening for a change, relative to a control in a statistically significant number of subjects, in the extent of epigenetic change within (i) two or more of (a) an intron; (b) an intron/exon boundary; and/or (c) a splicing region; and/or (ii) approximately one seventh or greater of an intron including an intron/exon boundary and/or a splicing region; and/or (iii) an intragenic region of a genetic locus in combination with an expansion mutation, the epigenetic change including extent of methylation of CpG and/or CpNpG sites located within the intron, intron/exon boundary and/or splicing region wherein a change in extent of epigenetic modification is indicative of the presence or severity of the pathological condition or a propensity to develop same.

Contemplated herein is a method of identifying a methylation or other epigenetic profile in a population of subjects indicative of a pathological condition associated with the FMR locus, the method comprising screening for a change, relative to a control, in a statistically significant number of subjects in the extent of epigenetic modification including extent of change in methylation of CpG and/or CpNpG sites within a region selected from:

(i) Fragile X-related Epigenetic Element 3 (1) [FREE3 (I)] in FMR1 comprising the nucleotide sequence set forth in SEQ ID NO:1 or a homolog thereof or portion or fragment thereof defined by having at least 80% nucleotide sequence identity to SEQ ID NO:1 or which hybridizes to SEQ ID NO:1 or its complementary form under medium stringency conditions;

(ii) intron 2 comprising the nucleotide sequence set forth in SEQ ID NO:2 or a homolog thereof or portion or fragment thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of the FMR1 gene or a homolog thereof or a portion or fragment thereof;

(iv) two or more of (a) an intron; (b) an intron/exon boundary; and/or (c) a splicing region within the FMR genetic locus;

(v) approximately one 7^{th} seventh or greater of an intron including an intron/exon boundary and/or a splicing region within the FMR genetic locus; and

(vi) a site within the FREE2 portion of intron 1;

wherein a change in extent of epigenetic modification is indicative of the presence of the pathological condition or a propensity to develop same in the population.

In accordance with this method, the assay may comprise the further step of determining the extent of a nucleotide expansion such as a (CGG)n expansion such as by PCR and/or Southern blot analysis.

Aspects herein extend to the use of the epigenetic profile of an intron within a genetic locus to determine the status, prognosis or disease development or recovery and/or treatment options including responsiveness of the subject to pharmacological agents and/or behavioral intervention strategies.

Computer programs to monitor changes in epigenetic modification or profile over time that may assist in making decisions regarding treatment options including responsiveness of the subject to pharmacological agents and/or behavioral intervention strategies, are also enabled herein.

Accordingly, another aspect provides a method of allowing a user to determine the status, prognosis and/or treatment response of a subject with respect to an FMR locus-associated pathology, the method including:

(a) receiving data in the form of extent of methylation or other epigenetic modification at a site within (i) two or more of (a) an intron; (b) an intron/exon boundary; and/or (c) a splicing region; and/or (ii) approximately one 7^{th} (seventh) or greater of an intron including an intron/exon boundary and/or a splicing region; and/or (iii) an intragenic region of a genetic locus in combination with an expansion mutation associated with the pathology, wherein the extent of methylation or other epigenetic modification provides a correlation to the presence, state, classification or progression of the pathology;

(b) transferring the data from the user via a communications network;

(c) processing the subject data via multivariate or univariate analysis to provide a disease index value;

(d) determining the status of the subject in accordance with the results of the disease index value in comparison with predetermined values; and

(e) transferring an indication of the status of the subject to the user via the communications network.

In an embodiment, contemplated herein is a method of allowing a user to determine the status, prognosis and/or treatment response of a subject with respect to an FMR locus-associated pathology, the method including:

(a) receiving data in the form of extent of methylation or other epigenetic modification at a site selected from:
(i) Fragile X-related Epigenetic Element 3 (I) [FREE3 (I)] in FMR1 comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;
(ii) intron 2 of FMR1 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions; and
(iii) an intron, intron/exon boundary and/or splicing region downstream of intron 2 in the FMR1 gene or a homolog thereof or a portion or fragment thereof;
(iv) two or more of (a) an intron; (b) an intron/exon boundary; and/or (c) a splicing region within the FMR genetic locus;
(v) approximately one seventh or greater of an intron including an intron/exon boundary and/or a splicing region; and
(vi) a site within the FREE2 portion of intron 1;
   wherein the extent of methylation or other epigenetic modification provides a correlation to the presence, state, classification or progression of the pathology;

(b) transferring the data from the user via a communications network;

(c) processing the subject data via multivariate or univariate analysis to provide a disease index value;

(d) determining the status of the subject in accordance with the results of the disease index value in comparison with predetermined values; and

(e) transferring an indication of the status of the subject to the user via the communications network.

A further embodiment enabled herein is a kit comprising primers which amplify regions of the FMR genetic locus, comprising CpG and/or CpNpG sites located within a region selected from:

(i) Fragile X-related Epigenetic Element 3 (I) [FREE3 (I)] in FMR1 comprising the nucleotide sequence set forth in SEQ ID NO:1 or a homolog thereof or portion or fragment thereof defined by having at least 80% nucleotide sequence identity to SEQ ID NO:1 or which hybridizes to SEQ ID NO:1 or its complementary form under medium stringency conditions;

(ii) intron 2 of FMR1 comprising the nucleotide sequence set forth in SEQ ID NO:2 or a homolog thereof or portion or fragment thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of FMR1 or a homolog thereof or a portion or fragment thereof;

(iv) two or more of (a) an intron; (b) an intron/exon boundary; and/or (c) a splicing region within the FMR genetic locus;

(v) approximately one 7^{th} (seventh) or greater of an intron including an intron/exon boundary and/or a splicing region; and

(vi) a site within the FREE2 portion of intron 1;

in the manufacture of a diagnostic kit or device to detect epigenetic modification of the FMR locus-associated with a pathological condition.

In an embodiment, the epigenetic modification relates to extent of, or change in, methylation at CpG and/or CpNpG sits within the selected regions of the FMR genetic locus, defined as FREE 3 (I), intron 2 and an intron, intron/exon boundary and/or splicing region downstream of intron 2, when in combination with an FM, or the FREE2 portion of intron 1 of the FMR1 gene. An embodiment associates hypermethylation at a site within FREE2 portion of intron 1 of the FMR1 gene with cognitive impairment. In an embodiment, this aspect applies to female subjects having a FM.

It is taught herein that FM and PM individuals have a significant relationship between methylation of specific CpG sites within the FREE regions proximal to the 3′ and 5′ epigenetic boundaries and learning and behavioral problems including co-morbid autism and that these epigenetic changes are consistent between different tissues and over a lifetime. It is also taught herein that these epigenetic changes are related to abnormal or lack of binding of a CCCTC-binding factor (CTCF) in the proximity of epigenetic boundaries in FM and PM individuals resulting in abnormal FMR1/ASFMR1 expression. This is the first functional evidence for long range epigenetic modification specific to FM and PM alleles and enables avenues for earlier diagnosis, treatment and intervention in individuals with an abnormal FMR1 gene.

In an embodiment, the primers useful in practicing the subject assay are selected from the list consisting of SEQ ID NOs:6 through 11. Those sequences include tag sequences. The present disclosure extends to the primer only portions of SEQ ID NOs:6 through 11. SEQ ID NOs:43 through 49 represent the nucleotide sequences of FREE2(A) 5′ Intr1 Amp 5, FREE2(B) 5′ Intr 1 PP2, FREE2(C) 4′ Intr 1 PP3, FREE2(D) 5′ Intr 1 PP4, 2CpGs, FREE2(E) 5′ Intr 1 PP6 and ASFMR1.

TABLE 1
Summary of sequence identifiers

| SEQUENCE | |
|---|---|
| ID NO: | DESCRIPTION |
| 1 | Nucleotide sequence of FREE3 (I) within the FMR1 gene |
| 2 | Nucleotide sequence of intron 2 of the FMR1 gene |
| 3 | Nucleotide sequence of intron 1 of FMR1 gene |
| 4 | Nucleotide sequence of FREE2 (B) |
| 5 | Nucleotide sequence of FREE2 (C) |
| 6 | Forward primer and tag sequence for FREE2 (B) |
| 7 | Reverse primer and tag sequence for FREE2 (B) |
| 8 | FREE 2 (C) forward primer and tag |
| 9 | FREE2 (C) reverse primer and tag |
| 10 | FREE3 forward primer and tag |
| 11 | FREE3 reverse primer and tag |
| 12 | Nucleotide sequecne of regulatory motif GATA-1 |
| 13 | Nucleotide sequence of regulatory motif HSF2 |
| 14 | Nucleotide sequence of regulatory motif C/EBP |
| 15 | Nucleotide sequence of regulatory motif CdxA |
| 16 | Nucleotide sequence of regulatory motif AML-1a |
| 17 | Nucleotide sequence of regulatory motif AML-1a |
| 18 | Nucleotide sequence of regulatory motif CdxA |
| 19 | Nucleotide sequence of regulatory motif CdxA |
| 20 | Nucleotide sequence of regulatory motif CdxA |
| 21 | Nucleotide sequence of regulatory motif HFH-1/HFH-2 |
| 22 | Nucleotide sequence of regulatory motif Cdx2 |
| 23 | Nucleotide sequence of regulatory motif SRY |
| 24 | Nucleotide sequence of regulatory motif SRY |
| 25 | Nucleotide sequence of regulatory motif SRY |
| 26 | Nucleotide sequence of regulatory motif S8 |
| 27 | Nucleotide sequence of regulatory motif SRY |
| 28 | Nucleotide sequence of regulatory motif CdxA |
| 29 | Nucleotide sequence of regulatory motif Oct-1 |
| 30 | Nucleotide sequence of intron1 downstream of FREE2 (C) |
| 31 | Nucleotide sequence of exon2 upstream of FREE3 |
| 32 | Nucleotide sequence of CGG amplification primer (r) |
| 33 | Nucleotide sequence of CGG amplification primer (f) |
| 34 | ASFMR1 (−1) forward primer |
| 35 | ASFMR1 (−1) reverse primer |
| 36 | ASFMR1 (−1) probe |
| 37 | ASFMR1 (−2) forward primer |
| 38 | ASFMR1 (−2) reverse primer |
| 39 | ASFMR1 (−2) probe |
| 40 | ASFMR1 (−3) forward primer |
| 41 | ASFMR1 (−3) reverse primer |
| 42 | ASFMR1 (−3) probe |
| 43 | Nucleotide sequence of FREE2(A) 5′ Intr1 Amp 5 |
| 44 | Nucleotide sequence of FREE2(B) 5′ Intr 1 PP2 |
| 45 | Nucleotide sequence of FREE2(C) 4′ Intr 1 PP3 |
| 46 | Nucleotide sequence of FREE2(D) 5′ Intr 1 PP4 |
| 47 | Nucleotide sequence of 2CpGs |
| 48 | Nucleotide sequence of FREE2(E) 5′ Intr 1 PP6 |
| 49 | Nucleotide sequence of ASFMR1 |

A list of abbreviations used herein is provided in Table 2.

TABLE 2
Abbreviations

| ABBREVIATION | DESCRIPTION |
|---|---|
| Ab | Antibody |
| ASFMR1 | Antisense Fragile X mental retardation 1 gene |
| CCCTC | CCCTC-binding factor |
| ChIP-seq | Chromatin immunoprecipitation sequencing |
| (CGG)ₙ | CGG repeat element located within 5′ untranslated |
| | region of the FMR1 gene |
| CpG | Cytosine and guanine separated by a phosphate |
| | (C-phosphate-G), which links the two nucleosides |
| | together in DNA |
| CpNpG | Cytosine and guanine separated by a nucleotide |
| | (N) where N is any nucleotide but guanine. The |
| | cytosine and N nucleotide are phosphorylated. |
| CVS | Cultured or uncultured Chorionic Villi Sample |
| DM | Mytotonic dystrophy |
| DNA | Deoxyribonuceic acid |
| DRPLA | dentatorubropallid oluysiantrophy |
| FIQ | Full scale IQ |
| FM | Full Mutation |
| FMR | Fragile X mental retardation genetic locus |
| | comprising of FMR1 and FMR4 genes |
| FMR1 | Fragile X mental retardation 1 gene |
| FMRP | Fragile X mental retardation protein |
| FRA12A | Fragile type, folic acid type, rare 12 |
| FRAXE | Fragile X E mental retardation |
| FRDA | Friedrich's ataxia |
| FREE | Fragile X related Epigenetic Element (e.g. |
| | FREE2 and FREE3(I))) |
| FREE3 | ASMR1 promoter |
| FXPOI | Fragile X-associated primary ovarian |
| | insufficiency |
| FXS | Fragile X Syndrome |
| FXTAS | Fragile X-associated Tremor Ataxia Syndrome |
| GZ | Gray Zone |
| HD | Huntington's disease |
| HRM | Heat Resolution Melt |
| MR | Mental retardation |
| ORF | Open Reading Frame |
| PCR | Polymerase Chain Reaction |
| PIQ | Performance IQ |
| PM | Premutation |
| POF | Premature Ovarian Failure |
| PolyQ | Polyglutamine |
| SBMA | spinobullar muscular atrophy (Kennedy disease) |
| SCA1 | spinocerebellar ataxia Type 1 |
| SCA17 | spinocerebellar ataxia Type 17 |
| SCA2 | spinocerebellar ataxia Type 2 |
| SCA3 | spinocerebellar ataxia Type 3 |
| SCA6 | spinocerebellar ataxia Type 6 |
| SCA7 | spinocerebellar ataxia Type 7 |
| SCA8 | spinocerebellar ataxia Type 8 |
| VIQ | Verbal IQ |

### BRIEF, DESCRIPTION OF THE FIGURES

Some figures contain color representations or entities. Color photographs are available from the Patentee upon request or from an appropriate Patent Office. A fee may be imposed if obtained from a Patent Office.

FIG. 1A is diagrammatic representation of the DNA map of the intron and exon regions 5′ and 3′ of the FMR1 CGG expansion (sequence numbering from GenBank L29074 L38501) in relation to FMR1 and ASFMR1 transcription start sites (the broken lines indicated spliced out regions), FMR1 promoter, the Fragile X-related epigenetic elements 1 and 2 (FREE 1 and 2), the FMR1 CpG island and methylation sensitive restriction sites (NruI, EagI and BssHII) analyzed using routine Fragile X Southern blot testing. A CGG repeat is located within the 5′ (UTR) of the FMR1 gene. ASFMR1 spans the CGG expansion in the antisense direction and is regulated by two promoters, with promoter one located within the CpG island (yellow box) and promoter located in the intron 2 of FMR1 (light pink box). The FREE2 located downstream of the CGG expansion. CTCF binding sites are located on either side of the CGG expansion (indicated by purple boxes). Primers utilized for MALDI-TOF methylation analysis targeted 6 regions at the Xq27.3 locus designated as FREE1, FREE2(A) described as amplicon 5 in (Godler et al. (2010) Hum Mol Genet 19(8):1618-1632); FREE2(B) FREE2(C); FREE2(D); FREE2(E), and intragenic ASFMR1 promoter (color coded) [FREE3].

FIG. 1B is a representation of the sequences amplified by primers utilized for MALDI-TOF methylation analysis targeted 6 regions at the Xq27.3 locus designated as FREE2(A) (described as amplicon 5 in Godler et al. (2010) supra; HMG); FREE2(B); FREE2(C); FREE2(D); FREE2(E), FREE2(F) and FREE3/ASFMR1 promoter (color coded). Individual CPG sites within each region are numbered accordingly. Prominent transcription factor binding sites and methylation sensitive restriction enzyme recognition sites are indicated in capital font, and are listed/identified in Table 1. << Indicates ASFMR1 transcription start site. The red arrow indicates the FREE2 3′ Boundary located at CpG1 of FREE2(E) which is underlined in the sequence. Text highlighted in pink indicates CTCF binding sites from UCSF Chip-Seq: CTCF site #1 binds FREE2(B) CpG1 to CpG32; CTCF site #2 binds FREE2(D) CpG5-7.

FIGS. 2A through C are photographic and graphical representations showing FMR1 mRNA and FMRP expression and FMR1 intron methylation in lymphoblast cell lines from normal range controls and FXS individuals. (A) FMRP and (B) FMR1 mRNA expression and (C) FMR1 intron 1 methylation analysis using SEQUENOM mass spectrometry assays, color coded (see FIG. 1 for sequence location), were examined in the cells from the same wells. FMR1 5′ and 3′ mRNA levels and FMRP expression were assessed using real-time PCR and western blot analysis, respectively. (A) The full size and truncated FMRP were expressed in controls and absent in these FM cell lines from individuals with clinical FXS. (B) FMR1 5′ and 3′ mRNA were absent in the cell lines from FXS males with 490, 530 and 543-633CGG alleles as well as a FM female carrier of 47 and 563 alleles, all affected with clinical FXS and hypermethylation at the CpG island (assessed using Southern blot). (C) the FMR1 intron 1 sequences were hypermethylated in FXS cell lines, while the intron was hypomethylated in all sites examined up to the 3′ epigenetic boundary (red arrow).

FIG. 3 is a diagrammatic representation of the intron and exon regions at the Xq27.3 locus (sequence numbering from GenBank L29074 L38501), locations of FMR1 and ASFMR1 transcription start sites and alternative splicing events. The locations of target sequences for FMR1 and ASFMR1 real-time PCR assays used are also indicated: ASFMR1 (−1) real-time assay: detects unspliced and splice variant C (positioned −282 to −343 from FMR1 transcription start site), ASFMR1 (−2) real-time assay: detects unspliced only (positioned −588 to −663 from FMR1 transcription start site), ASFMR1 real-time assay: detects all (positioned −1299 to −1360 from FMR1 transcription start site).

FIGS. 4A through C are graphical representations of different FMR1 and ASFMR1 transcripts in RNA samples from lymphoblast lines of 6 male controls, two FXS males (samples 849 and 862) and one FXS female (865) (described in FIG. 2). The control and FXS RNA samples were either treated with TURBO DNase (A), RQ1 DNase (B), RNase A (C), or were untreated. Addition of TURBO DNase or RQ1 DNAse buffers to RNA samples without DNase were included as additional controls in A and B. The FMR1 and ASFMR1 transcripts were quantified using real-time RT-PCR relative standard curve method; normalized to mRNA levels of three internal control genes, GUS, GAPDH and B2M. FMR15′ and 3′ assays showed no signal for the FXS RNA samples, while similar levels were detected in all control samples (upper two panels in A, B and C). TURBO and RQ1 DNAse treatment caused ˜50% decrease in the FMR1 levels in most of the control samples; while RNase A treatment caused complete loss of FMR1 and ASFMR1 signals. While decrease of ASMFR1 (−1)(−2) and (−3) levels was also observed in all control samples caused by TURBO and RQ1 DNAse treatment, in FXS samples (with analogous to control ASFMR1 levels in the untreated samples) TURBO and RQ1 DNAse treatment resulted in complete loss of signal for all three ASFMR1 assays. Because DNase can only degrade RNA molecules if they form complexes with DNA, this suggests that ASFMR1 RNA forms RNA:DNA complexes more readily in FXS samples than in controls. Increase in RNA:DNA interaction of ASFMR1 in FXS may lead to methylated FMR1 promoter and adjacent regions (FIG. 1) and silencing FMR1 expression leading to loss of FMRP and the resulting FXS clinical phenotype.

FIG. 5 is a graphical representation showing full scale IQ (FIQ) and FREE2 methylation in the blood of human female subjects. (A) The difference in FREE2 methylation output ratio (y axis) between normal CGG size controls (NC), PM carriers with FIQ>70, FM carriers with FIQ>70, and FM carriers with FIQ<70 (x axis). (B) Nonparametric Spearman correlation between FREE2 methylation (y axis) and FIQ (x axis) in FM only females.

FM FIQ<70 compared to HC ***-p<0.0001; **-p<0.001; *-p<0.05;
FM IQ<70 compared to PM IQ>70 ♦♦♦-p<0.0001; ♦♦-p<0.001; ♦-p<0.05;
FM IQ<70 compared to FM IQ>70 ###-p<0.0001; ##-p<0.001; #-p<0.05
FM IQ>70 compared to HC □□□-p<0.0001; □□-p<0.001; □-p<0.05
FM IQ>70 compared to PM IQ>70 ▪▪▪-p<0.0001; ▪▪-p<0.001; ▪-p<0.05
PM IQ>70 compared to HC ⋄⋄⋄-p<0.0001; ⋄⋄-p<0.001; ⋄-p<0.05

FIG. 6 is a graphical representation showing verbal IQ (VIQ) and FREE2 methylation in the blood of human female subjects. (A) The difference in FREE2 methylation output ratio (y axis) between normal CGG size controls (NC), PM carriers with VIQ>70, FM carriers with VIQ>70, and FM carriers with VIQ<70 (x axis). (B) Nonparametric Spearman correlation between FREE2 methylation output ratio (y axis) and VIQ (x axis) in FM only females.

FM VIQ<70 compared to HC ***-p<0.0001; **-p<0.001; *-p<0.05;
FM VIQ<70 compared to PM VIQ>70 ♦♦♦-p<0.0001; ♦♦-p<0.001; ♦-p<0.05;
FM VIQ<70 compared to FM VIQ>70 ###-p<0.0001; ##-p<0.001; #-p<0.05
FM VIQ>70 compared to HC-p<0.0001; -p<0.001; -p<0.05
FM VIQ>70 compared to PM IQ>70-p<0.0001; -p<0.001; -p<0.05
PM VIQ>70 compared to HC ⋄⋄⋄-p<0.0001; ⋄⋄-p<0.001; ⋄-p<0.05

FIG. 7 is a graphical representation of performance IQ (PIQ) and FREE2 methylation in the blood of human female subjects. (A) the difference in FREE2 methylation output ratios (y axis) between normal CGG size controls (NC), PM carriers with FIQ>70, FM carriers with FIQ>70, and FM carriers with PIQ<70 (x axis). (B) Nonparametric Spearman correlation between FREE2 methylation output ratio (y axis) and PIQ (x axis) in FM only females.

FM PIQ<70 compared to HC ***-p<0.0001; **-p<0.001; *-p<0.05;
FM PIQ<70 compared to PM PIQ>70 ♦♦♦-p<0.0001; ♦♦-p<0.001; ♦-p<0.05;
FM PIQ<70 compared to FM PIQ>70 ###-p<0.0001; ##-p<0.001; #-p<0.05
FM PIQ>70 compared to HC-p<0.0001; -p<0.001; -p<0.05
FM PIQ>70 compared to PM PIQ>70-p<0.0001; -p<0.001; -p<0.05 Stats to do
PM PIQ>70 compared to HC ⋄⋄⋄-p<0.0001; ⋄⋄-p<0.001; ⋄-p<0.05

FIG. 8 is a graphical representation showing comparison of methylation at the Exon1/Intron 1 border and the 3′ epigenetic boundary in 15 control (HC), 18 premutation (PM), 4 ‘high functioning’ full mutation (IQ>70; UFM) males and 41 FM males with cognitive impairment (IQ<70; FXS). (A) CpG10 to 12 and the 3′ epigenetic boundary region (E) CpG1 to 4. Note: all comparison showed P<0.001. Note: FXS compared to HC ***; FXS compared to PM ###; FXS compared to UFM ̂̂̂; PM compared to HC >>>.

FIGS. 9A and B are graphical representations showing FREE2 pG10-12 methylation in newborn blood spots (NBS), dried blood spots (DBS) and fresh blood. (A) methylation measures in NBS and fresh blood of FXS males compared to control and PM individuals. (B) repeated methylation measures for five FXS malesin NBS at birth and in DBS made at 3, 4, 5, 35 and 48 years of age with corresponding CGG sizes indicated in brackets. Note: broken line—the cut off methylation thresholds for FM females; ♂ FXS NBS compared to ♂ FXS fresh blood DNA *-p<0.05; ♂ FXS NBS compared to ♂ control fresh blood DNA ♦♦♦-p<0.001; ♂ FXS NBS compared to ♂ PM fresh blood DNA ###-p<0.001.

### DETAILED DESCRIPTION

Taught herein is a method for identifying an epigenetic profile of an intron, intron/exon boundary and/or splicing region within a genetic locus associated, indicative, instructive or informative of a pathological condition. The epigenetic modification occurs in:

(i) two or more of (a) an intron; (b) an intron/exon boundary; and/or (c) a splicing region;

(ii) approximately one 7^{th} seventh or greater of an intron including an intron/exon boundary and/or a splicing region; and/or

(iii) an intragenic site in combination with an expansion mutation;

within a genetic locus.

By “epigenetic profile” includes epigenetic modifications such as methylation including hypermethylation, RNA/DNA interactions, expression profiles of non-coding RNA, histone modification, changes in acetylation, obiquitylation, phosphorylation, sumoylation, activation or deactivation, chromatin altered transcription factor levels and the like. Particularly, the extent of methylation, RNA/DNA interaction and non-coding RNA expression are determined as well as any changes therein.

The pathological condition may be a neurological or non-neurological condition. Insofar as the condition is neurological, it may be described as a neuropathological condition or a pathoneurological condition which encompasses neurodegenerative and neurodevelopmental disorders. Non-neurological pathologies are also contemplated herein as well as any nucleotide expansion disease or condition.

Conditions and disorders contemplated herein include polyglutamine (polyQ) diseases such as Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA7), spinocerebella ataxia Type 17 (SCA17) and non-polyQ diseases such as Fragile X syndrome (FXS), Fragile X-associated tremor or ataxia (FXTAS), Fragile XE mental retardation (FRAXE), Fragile type, folic acid type, rare 12 (FRA12A), Friedrich's ataxia (FRDA), myotonic dystrophy (DM), spinocerebella ataxia (SCA8) and spinocerebella ataxias Type 12 (SCA12). Other conditions contemplated herein include premutation-related disorders including Fragile X-associated primary ovary insufficiency (FXPOI), autism (including co-morbid autism), mental retardation (MR), Klinefelter's syndrome, RNA toxicity disease, Turner's syndrome, a modified X-chromosome and cognitive impairment. Other conditions include learning and behavioral problems. The present disclosure particularly identifies nucleotide expansion diseases and conditions and FMR genetic locus-associated pathology conditions.

In another embodiment, a method is enabled for identifying an epigenetic profile in a genome of a cell indicative of a pathological condition selected from Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA7), spinocerebella ataxia Type 17 (SCA17), Fragile X-associated tremor or ataxia (FXTAS), Fragile XE mental retardation (FRAXE), FXPOI, Friedrich's ataxia (FRDA), Fragile type, folic acid type, rare 12 (FRA12A), myotonic dystrophy (DM), spinocerebella ataxia (SCA8) and spinocerebella ataxias Type 12 (SCA12), autism (including co-morbid autism), mental retardation, Klinefelter's syndrome, RNA toxicity disease, Turner's syndrome and a modified X-chromosome and cognitive impairment, the method comprising screening for a change relative to the control in the extent of epigenetic modification within (i) two or more of (a) an intron; (b) an intron/exon boundary; and/or (c) a splicing region; and/or (ii) approximately one seventh or greater of an intron including an intron/exon boundary and/or a splicing region; and/or (iii) an intragenic region of a genetic locus in combination with an expansion mutation associated with the pathological condition wherein the extent of epigenetic change is indicative of the presence or severity of the pathological condition or a propensity to develop same. This method also applies to detecting learning and behavioral problems.

Particular conditions include Fragile X-associated tremor or ataxia (FXTAS), Fragile XE mental retardation (FRAXE), premutation-related conditions including but not limited to FXPOI, Friedrich's ataxia (FRDA), Fragile type, folic acid type, rare 12 (FRA12A), autism (including co-morbid autism), mental retardation, Klinefelter's syndrome, RNA toxicity disease, Turner's syndrome, a modified X-chromosome and cognitive impairment.

In relation to this aspect, an “intron”, “intron/exon boundary” and “splicing region”, are regarded as an intron, intron/exon boundary and splicing region within a genetic locus or a gene within a genome. The intron, intron/exon boundary and splicing region may also encode a regulatory RNA species.

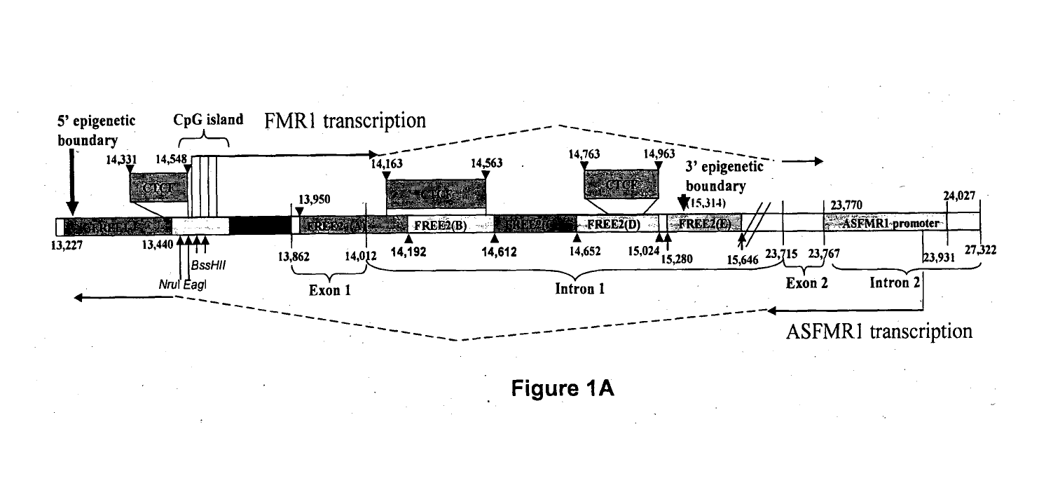

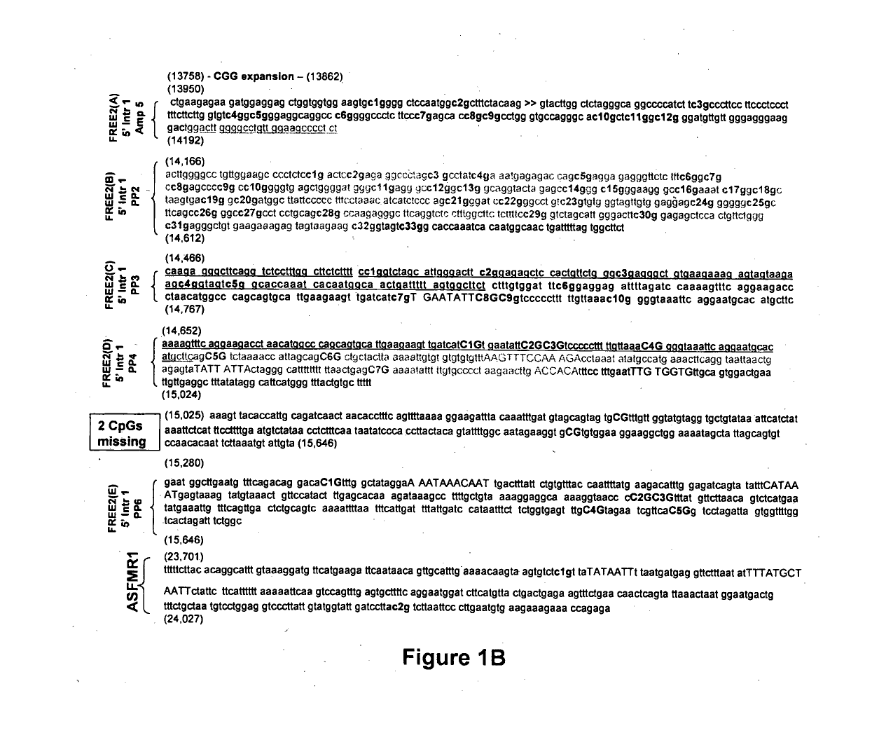

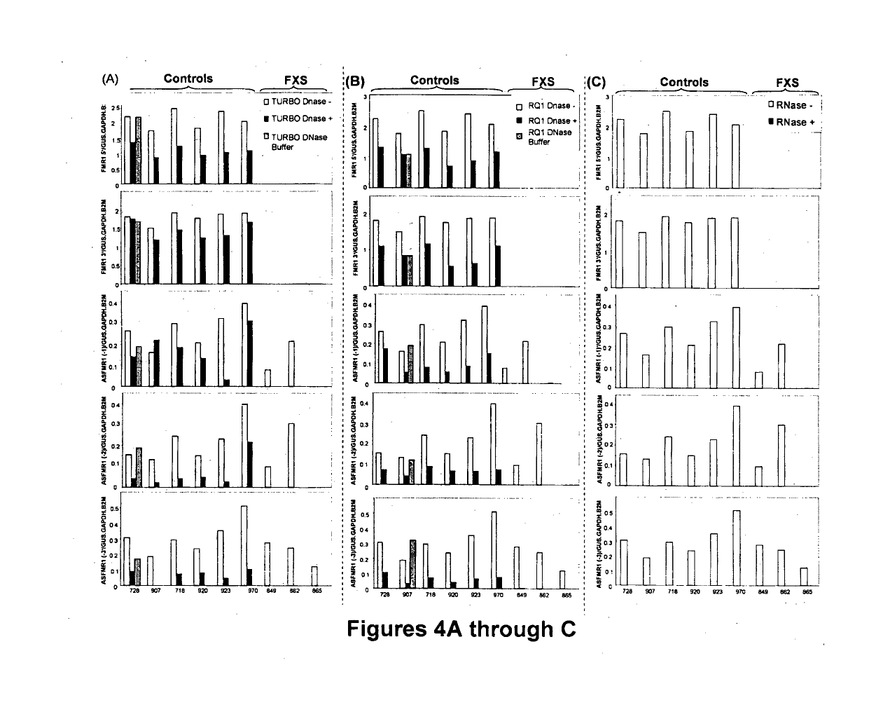

In an embodiment, the pathological condition is associated with an epigenetic profile of the FMR genetic locus. For the purposes of the present disclosure, the “FMR genetic locus” includes the FMR1, FMR4 and ASFMR1 genes as well as promoter and regulatory regions and introns and exons. The FMR genetic locus comprises a promoter region, a (CGG)ₙ region proximal to the promoter and exonic and intronic regions of the FMR1, FMR4 and ASFMR1 genes as depicted in FIGS. 1A and 4A. The promoter is generally referred to as the “FMR1 promoter”. The FMR locus includes introns, intron/exon boundaries and splicing regions wherein it is proposed herein that epigenetic changes occur within (i) two or more of (a) an intron; (b) an intron/exon boundary; and/or (c) a splicing region; and/or (ii) approximately one seventh or greater of an intron including an intron/exon boundary and/or a splicing region; and/or (iii) with the FREE2 portion of intron 1 of the FMR1 gene in combination with an FM which are indicative or diagnostic of a pathological condition or its severity involving the FMR1, FMR4 and/or ASFMR1 genes. FREE3(I) is further defined below. “FREE2” means the portion of FREE2(A), FREE2(B) and/or FREE2(C) within intron 1 of the FMR1 gene including an intron/exon boundary.

In another embodiment, a method is contemplated for identifying an epigenetic profile in a genome of a cell indicative of a pathological condition selected from Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA7), spinocerebella ataxia Type 17 (SCA17), Fragile X-associated tremor or ataxia (FXTAS), Fragile XE mental retardation (FRAXE), FXPOI, Friedrich's ataxia (FRDA), Fragile type, folic acid type, rare 12 (FRA12A), myotonic dystrophy (DM), spinocerebella ataxia (SCA8) and spinocerebella ataxias Type 12 (SCA12), autism (including co-morbid autism), mental retardation, Klinefelter's syndrome, RNA toxicity disease, Turner's syndrome, a modified X-chromosome and cognitive impairment, the method comprising screening for a change relative to the control in the extent of epigenetic modification within (i) two or more of (a) an intron; (b) an intron/exon boundary; and/or (c) a splicing region; or (ii) approximately one seventh or greater of an intron including an intron/exon boundary and/or a splicing region; and/or (iii) an intragenic region of a genetic locus in combination with an expansion mutation associated with the pathological condition wherein the extent of epigenetic change is indicative of the presence or severity of the pathological condition or a propensity to develop same.

Particular conditions include Fragile X-associated tremor or ataxia (FXTAS), Fragile XE mental retardation (FRAXE), premutation-related conditions including but not limited to FXPOI, Friedrich's ataxia (FRDA), Fragile type, folic acid type, rare 12 (FRA12A), autism (including co-morbid autism), mental retardation, Klinefelter's syndrome, RNA toxicity disease, Turner's syndrome, a modified X-chromosome and cognitive impairment.

In an embodiment, the epigenetic change is in the FMR genetic locus and in particular within an intron, intron/exon boundary and/or splicing region downstream of the FMR genetic locus including the FMR1 gene. In another embodiment, the epigenetic change is hypermethylation of a site within the FREE2 portion of intron 1 of the FMR1 gene wherein hypermethylation of one or more sites within FREE2 is associated with cognitive impairment. In an embodiment, the cognitive impairment is in a female human subject. In an embodiment, the human female subject presents with a full mutation (FM); i.e. >200 CGG repeats.

Hence, the present disclosure teaches the manufacture of an assay to identify an epigenetic profile of an FMR genetic locus-associated pathological condition.

The FMR genetic locus is depicted in part in FIGS. 1A and 4A. The present disclosure is predicated in part on a determination of the methylation or other epigenetic signature of introns, intron/exon boundaries and/or splicing signals or part within the FMR genetic locus and in particular the FMR1 gene. The nucleotide sequence of intron 1 of the FMR1 gene is set forth in SEQ ID NO:3. The present disclosure extends to homologs of a gene such as genetic loci comprising a nucleotide sequence at least 80% identical to SEQ ID NO:3 or a nucleotide sequence capable of hybridizing to SEQ ID NO:3 under medium stringency conditions.

A useful region taught herein in accordance with the present disclosure is intron 2 (SEQ ID NO:2) and a portion of intron 2 is termed herein “FREE3 (I)” and is defined by SEQ ID NO:1 within the FMR1 gene. The present disclosure extends to any intron, intron/exon boundary and splicing region within the FMR1 gene including (i) two or more of (a) an intron; (b) an intron/exon boundary; and/or (c) a splicing region; and/or (ii) approximately one seventh or greater of an intron including an intron/exon boundary and/or a splicing region as well as nucleotide sequences having at least 80% identity to any of these regions or a nucleotide sequence capable of hybridizing to these sequences or their complementary forms under medium stringency conditions. The present disclosure extends to portions and fragments of these regions. The present disclosure also teaches hypermethylation or other epigenetic changes in one or more sites of the FREE2 portion of intron 1 of the FMR1 gene in combination with an FM. The present disclosure further teaches isolated nucleotide sequences corresponding to these regions. Without limiting the present disclosure to any one theory or mode of action, epigenetic changes in these introns may affect the ability of the introns, intron/exon boundaries and/or splicing regions to transcribe regulatory RNAs which in turn have an effect on transcription capability.

It is taught herein that FM and PM individuals have a significant relationship between methylation of specific CpG sites within the FREE regions proximal to the 3′ and 5′ epigenetic boundaries and learning and behavioral problems including co-morbid autism and that these epigenetic changes are consistent between different tissues and over a lifetime. It is also taught herein that these epigenetic changes are related to abnormal or lack of binding of a CCCTC-binding factor (CTCF) in the proximity of epigenetic boundaries in FM and PM individuals resulting in abnormal FMR1/ASFMR1 expression. This is the first functional evidence for long range epigenetic modification specific to FM and PM alleles and enables avenues for earlier diagnosis, treatment and intervention in individuals with an abnormal FMR1 gene.

Hence, an aspect provides a method for identifying a pathological condition in a mammalian subject including a human, the method comprising screening for a change relative to a control in the extent of epigenetic modification within a region selected from:

(i) Fragile X-related Epigenetic Element 3 (I) [FREE3 (I)] in FMR1 comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;

(ii) intron 2 of FMR1 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of FMR1 or a homolog or a portion or fragment thereof;

(iv) two or more of (a) an intron; (b) an intron/exon boundary; and/or (c) a splicing region within the FMR genetic locus;

(v) approximately one seventh or greater of an intron including an intron/exon boundary and/or a splicing region; and

(vi) a site within the FREE2 portion of intron 1;

wherein a change in extent of genetic modification relative to a control is indicative of the presence or severity of the pathological condition or a propensity to develop same.

The present disclosure enables a method for identifying in a genome of a mammalian cell including a human cell, a pathological condition associated with methylation and other epigenetic change within the FMR locus, the method comprising extracting genomic DNA from the cell and subjecting the DNA to an amplication reaction using primers selective of a region of the FMR genetic locus comprising CpG and/or CpNpG sites, the CpG and CpNpG sites located in a region selected from:

(i) Fragile X-related Epigenetic Element 3 (I) [FREE3 (I)] of FMR1 comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;

(ii) intron 2 of FMR1 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of FMR1 or a homolog thereof or a portion or fragment thereof;

(iv) two or more of (a) an intron; (b) an intron/exon boundary; and/or (c) a splicing region within the FMR genetic locus;

(v) approximately one seventh or greater of an intron including an intron/exon boundary and/or a splicing region; and

(vi) a site within the FREE2 portion of intron 1;

and subjecting the amplified DNA to a methylation or other epigenetic assay to determine the extent of epigenetic modification of the DNA wherein a change in extent of epigenetic modification relative to a control is indicative of the presence or severity of the pathological condition or propensity to develop same.

In an embodiment, the epigenetic modification is methylation of CpG and/or CpNpG sites and the assay identifies the extent of methylation change. This change may be an elevation or increase in methylation or a decrease in methylation relative to a control. Alternatively, the epigenetic modification is extent of change in RNA/DNA interaction and/or change in profile of expression of expression of non-coding RNA. Yet in another embodiment, the epigenetic profile is a change in histone modification, changes in acetylation, obiquitylation, phosphorylation, sumoylation, activation or deactivation, chromatin altered transcription factor levels and the like. In an embodiment, the epigenetic change is hypermethylation.

An aspect of the present disclosure teaches a method for diagnosing or predicting cognitive impairment in a subject, the method comprising screening for an enhanced epigenetic profile in the FREE2 portion of intron 1 of the FMR1 gene wherein the presence of enhanced epigenesis in combination with an FM or a CGG expansion approaching an FM is indicative of cognitive impairment or a risk of developing same.

In an embodiment, the subject is a human. In an embodiment, the human is a human female. In an embodiment, the human female presents with an expanded FMR1 gene anneal or is in the process of developing an FXS or passing on the expanded FMR1 anneal to offspring. The term “enhanced epigenetic profile” includes hypermethylation.

In accordance with the present disclosure, a method is provided wherein the extent of methylation or other epigenetic modification provides a quantitative or semi-quantitative or qualitative indication of extent of change in epigenetic profile in (i) two or more of (a) an intron; (b) an intron/exon boundary; and/or (c) a splicing region; and/or (ii) approximately one seventh or greater of an intron including an intron/exon boundary and/or a splicing region; and/or (iii) the FREE2 portion of intron 1 of the FMR1 gene in combination with an FM and as such the level of epigenetic modification defines the severity of the pathological condition alone or in combination with the extent of (CGG)ₙ expansion. The number of repeats indicate whether a subject is a healthy control or has a Gray Zone (GZ) pathology, premutation (PM) pathology or full mutation (FM) pathology. The method of the present disclosure may also be used in conjunction with other assays such as Southern blot or PCR to measure (CGG)ₙ expansion. An “intragenic region” includes the FREE2 portion of intron 1 of FMR1. Reference to “a site in an intragenic region” or a “site in FREE2” includes a single or multiple sites. Reference to “FREE2” includes FREE2(A), FREE2(B) and/or FREE2(C) insofar as they are located in intron 1 of the FMR1 gene.

The present disclosure is not limited to the FMR genetic locus and pathological conditions only associated therewith. Rather, the present disclosure teaches any epigenetic modification in any genetic locus selected from (i) two or more of (a) an intron; (b) an intron/exon boundary; and/or (c) a splicing region; and/or (ii) approximately one seventh greater of an intron including an intron/exon boundary and/or splicing region; and/or (iii) an intragenic region in combination with an expansion mutation and which epigenetic change is associated with a pathological condition.

By “approximately one seventh or greater” means from about 20% or greater or nucleotides capable of epigenetic change or modification have undergone a change. This includes 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 67, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% of the nucleotides.

As taught herein, a “pathological condition” or “disease condition” includes an abnormal condition including a neurodevelopmental condition or a neurodegenerative condition or a non-neurological condition as defined by objective or subjective manifestations of disease. The assay herein described is particularly useful for diagnosing nucleotide expansion diseases or conditions. The assay of enabled herein includes a genetic determination to be made to complement other symptom-based diagnoses such as based on behavioral studies or may be made in its own right. The assay may be part of a suit of diagnostic or prognostic genetic assays of embryos, pre- and post-natal subjects. A saliva test may also be conducted. A saliva test enables salival DNA to be analyzed. This also applies to a cheek sample. The terms “method”, “assay”, “system”, “test”, “determination”, “prognostic”, “diagnostic”, “report” and the like may all be used to describe the methylation assay of selected regions of the FMR genetic locus or other genetic locus. The epigenetic assay such as a methylation assay determines the epigenetic profile or extent of epigenetic change compared to a control which suggests or indicates or is instructive of a disease or condition associated with epigenetic modification of an intron within a genetic locus. The present assay is also useful in population, studies such as epidemiological studies including studies of ethnic populations.

Accordingly, the present disclosure further provides a method of identifying a methylation or other epigenetic profile in populations of subjects indicative of a pathological condition, the method comprising screening for a change relative to a control in a statistically significant number of subjects the extent of epigenetic modification in (i) two or more of (a) an intron; (b) an intron/exon boundary; and/or (c) a splicing region; and/or (ii) approximately one seventh or greater of an intron including an intron/exon boundary and/or a splicing region; and/or (iii) an intragenic region in combination with an expansion mutation; within a genetic locus wherein a change in extent of epigenetic modification is indicative of the presence or severity of the pathological condition or a propensity to develop same.

The present disclosure teaches a method of identifying a methylation or other epigenetic profile in a population of subjects indicative of a pathological condition associated with the FMR locus, the method comprising screening for a change, relative to a control, in a statistically significant number of subjects in the extent of epigenetic modification including extent of change in methylation of CpG and/or CpNpG sites within a region selected from:

(i) Fragile X-related Epigenetic Element 3 (I) [FREES (I)] in FMR1 comprising the nucleotide sequence set forth in SEQ ID NO:1 or a homolog thereof or portion or fragment thereof defined by having at least 80% nucleotide sequence identity to SEQ ID NO:1 or which hybridizes to SEQ ID NO:1 or its complementary form under medium stringency conditions;

(ii) intron 2 comprising the nucleotide sequence set forth in SEQ ID NO:2 or a homolog thereof or portion or fragment thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 of the FMR1 gene or a homolog thereof or a portion or fragment thereof;

(iv) two or more of (a) an intron; (b) an intron/exon boundary; and/or (c) a splicing region within the FMR genetic locus;

(v) approximately one 7^{th} seventh or greater of an intron including an intron/exon boundary and/or a splicing region; and

(vi) a site within the FREE2 portion of intron 1;

wherein a change in extent of epigenetic modification is indicative of the presence of the pathological condition or a propensity to develop same in the population.

In accordance with this method the assay may comprise the further step of determining the extent of (CGG)ₙ expansion such as by PCR and/or Southern blot analysis of bisulfite converted and/or non converted DNA. Furthermore, this assay may be conducted with one or more assays contemplated and described in International Patent Application NO. PCT/AU2010/000169 filed on 17 Feb. 2010, the contents of which are incorporated by reference in their entirety. For example, a subject may first be screened to ascertain if the subject has a FM, PM or GZ. Subjects, including female human subjects, with a FM may then be selected to ascertain the presence or absence of hypermethylation in one or more sites with the FREE2 portion of intron 1 of the FMR1 gene. The presence of hypermethylation within FREE2 is indicative of cognitive impairment or the potential of development of same when in combination with an FM or a CGG expansion approaching an FM.

In an embodiment, the extent of methylation or change in extent of methylation is detected and associated with the pathology condition such as but not limited to an expansion disease or condition.

An epigenetic map and in particular a methylation map of introns, intron/exon boundaries and/or splicing regions within the FMR locus has thus been constructed in accordance with the present disclosure using standard techniques such as high throughput mass spectrometry in the genome of various cells. Any cell or sample type of cell may be assayed. A sample type includes fresh and dried blood samples, salvial and buccal samples. These cells or sample types include cultured or uncultured Chorionic Villi Sample (CVS) cells, lymphoblasts, blood cells, dried adult or newborn blood spots, buccal cells, an amniocyte, EBV transformed lymphoblast cell lines and DNA from a salival swab or cheek sample from male and female subjects with either no symptoms or from a spectrum of a pathological condition such as Fragile X mental retardation symptoms. In an embodiment a Fragile X-related Epigenetic Element (FREE3 (I) has been identified within intron 2 of the FMR1 gene. It is proposed that this region [FREE3 (I)] or other regions of intron 2 or other introns or parts thereof including intron/exon boundaries and splicing regions downstream of intron 2 of FMR1 or elsewhere in the FMR genetic locus are responsible for the regulation of transcription of FMR4 and ASFMR1 and FMR1 and expression of FMRP. In another embodiment, hyper-epigenetic changes at one or more sites within the FREE2 portion of intron 1 of the FMR1 gene in human female subjects with an FM are likely to have or develop cognitive impairment. The latter can conveniently be measured in cells obtained from a blood test or a saliva DNA test.

In an embodiment, the present disclosure determines that the extent of methylation in CpG and/or CpNpG sites located within the region downstream of intron 1 or part thereof such as FREE3 (I) closely corresponds to a healthy condition or a level or severity of disease within the spectrum of PM to FM including GZ subjects such a correspondence may be in a further association with other epigenetic modifications within the FMR genetic locus and/or CGG expansion. Furthermore, using the methylation assay, methylation levels of the FREE3 (I) region provide fully quantitative results, which also reflect the degree of X-chromosome modification in females. This can be more informative than methylation patterns of a promoter region only, which may be biased due to its proximity to a nucleotide expansion, and hence can only provide a qualitative assessment of methylation.

Hence, in an embodiment, the present disclosure contemplates a change in extent of methylation which includes an increase or decrease in extent of methylation. There may also be no change in the extent of methylation within an intron of a genetic locus. However, the present disclosure extends to the detection of the change in extent of any epigenetic modification. Such a change or level of methylation in an intron is proposed to be associated with a pathological condition or its severity. In this context, an “intron” includes an intron/exon boundary and/or a splicing region.

In another embodiment, the present disclosure enables identification of subjects, including human female subjects, having or likely to develop cognitive impairment, the method comprising screening for increased epigenesis (including hypermethylation) in one or more sites within the FREE2 portion of intron 1 of the FMR1 gene in subjects with an FM or who are developing an FM wherein the presence of increased epigenesis is indicative of a subject with cognitive impairment or a likelihood of developing same.

A “normal” or “control” in the assay of the present disclosure may be a control genome from a healthy individual performed at the same time or the epigenetic pattern may be compared to a statistically validated standard. In relation to a nucleotide expansion disease condition, a healthy individual includes a subject with a nucleotide repeat within the normal range with no clinically apparent pathological phenotype. For example, in relation to (CGG)ₙ expansion conditions within the FMR genetic locus, this includes when n is <40.

The present disclosure also explores the relationship between transcription and epigenetic profile of introns or parts thereof and pathological conditions. A “part” includes an intron/exon boundary and splicing region. In an embodiment, methylated CpG sites are identified within FREE3 (I) or intron 2 or a FREE2 portion of intron 1 of the FMR1 gene in subjects with Fragile X mental retardation conditions including symptoms of cognitive impairment. In another embodiment, the methylated CpG sites are identified in (i) two or more of (a) an intron; (b) an intron/exon boundary; and/or (c) a splicing region; and/or (ii) approximately one seventh or greater of an intron including an intron/exon boundary and/or a splicing region; and/or (iii) in a site in FREE2 of intron 1 of the FMR1 gene in combination with an FM.

As used herein, the terms “subject”, “patient”, “individual”, “target” and the like refer to any organism or cell of the organism on which an assay of the present disclosure is performed whether for experimental, diagnostic, prophylactic, and/or therapeutic purposes. Typical subjects include both male and female humans but the present disclosure extends to experimental animals such as non-human primates, (e.g., mammals, mice, rats, rabbits, pigs and guinea pigs/hamsters). The “subject” may also be referred to as a population since the present disclosure is useful in populations studies including epidemiological studies or assays of ethnic population. In a particular embodiment, the subject is a human. The test may be tailored to human females or human males or pre-natal humans and may also be conducted in utero. In an embodiment, in relation to epigenetic changes in the FREE2 portion of intron 1 of the FMR1 gene, the subject includes a human female as well as a human female with an FM.

The terms “Fragile X mental retardation-like condition” and FMR condition” refer to a neurological disease, disorder and/or condition characterized by one or more of the following symptoms: (1) behavioral symptoms, including but not limited to hyperactivity, stereotypy, anxiety, seizure, impaired social behavior, learning and attention problems and/or cognitive delay; (2) defective synaptic morphology, such as an abnormal number, length, and/or width of dendritic spines; and/or (3) defective synaptic function, such as enhanced long-term depression (LTD); and/or reduced long-term potentiation (LTP); and/or impaired cognitive ability and/or behavioral deficiency. The pathological condition is a disease, disorder, and/or condition caused by and/or associated with epigenetic changes within an intron or part thereof within the FMR genetic locus such as downstream of intron 1 of the FMR1 gene. Such epigenetic changes may be alone or in combination with one or more of the following: (1) a mutation in FMR1 or FMR4 or ASFMR1; (2) defective FMR1/FMR4/ASFMR1 expression; (3) increased and/or decreased levels of FMRP; (4) defective FMRP function; (5) increased and/or decreased expression of genes or genetic functions regulated by FMR1, FMRP, FMR4 transcript or ASFMR1 transcript; (6) the increased methylation of FMR locus at CpG or CpNpG sites in the region upstream of FMR1 promoter and/or the region downstream of the (CGG)ₙ portion of the FMR1 promoter but not including the (CGG)ₙ portion; (7) an increased and/or decreased function of the FMR locus via miRNAs and/or members of the miRNA pathway; (8) an increased and/or decreased ability of FMRP to interact with its known target RNAs, such as RNAs encoding Rac1, microtubule-associated protein IB, activity-regulated cytoskeleton-associated protein, and/or alpha-calcium/calmodulin-dependent protein kinase II; (9) symptoms of FXS, FXTAS, FRA12A, FXPOI, mental retardation, autism and/or autism spectrum disorders (including co-morbid autism); and/or (10) cognitive impairment in a subject with an FM and hypermethylation in a FREE2 portion of intron 1 of the FMR1 gene. Those of ordinary skill in the art will appreciate that the teachings herein are applicable to any neurodevelopmental or neurodegenerative disorders linked, associated or otherwise influenced by the function of the FMR genetic locus or genes therein such as FMR1, FMR4 and ASFMR1. Disorders also contemplated herein include some tri-nucleotide expansion disorders.

Furthermore, the present disclosure extends to a range of nucleotide expansion disorders. Conditions and disorders contemplated herein include polyglutamine (polyQ) diseases such as Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA1), spinocerebella ataxia Type 17 (SCA17) and non-polyQ diseases such as Fragile X syndrome (FXS), Fragile X-associated tremor or ataxia (FXTAS), Fragile XE mental retardation (FRAXE), Friedrich's ataxia (FRDA), FXPOI and other premutation disorders, Fragile type, folic acid type, rare 12 (FRA12A), myotonic dystrophy (DM), spinocerebella ataxia (SCA8) and spinocerebella ataxias Type 12 (SCA12). Other conditions contemplated herein include autism, mental retardation, Klinefelter's syndrome, RNA toxicity disease, Turner's syndrome, a modified X-chromosome as well as certain tri-nucleotide expansion disorders, cognitive impairment, behavioral and learning problems.

The term “genomic DNA” includes all DNA in a cell, group of cells, or in an organelle of a cell and includes exogenous DNA such a transgenes introduced into a cell.

The present disclosure teaches the determination of the presence of an FMR genetic locus-associated pathology based on extent of methylation of CpG/CpNpG sites located within (i) an intron downstream of intron 1 of the FMR1 gene or part of an intron; (ii) two or more of (a) an intron; (b) an intron/exon boundary; (c) a splicing region; and/or (iii) approximately one seventh or greater of an intron including an intron/exon boundary and/or a splicing region; and/or (iv) one or more sites in the FREE2 portion of intron 1 of the FMR1 gene in combination with an FM. The downstream FMR1 introns may extend beyond the FMR1 gene. In a particular embodiment, the extent of methylation in part of intron 2 [FREE3 (I)] is identified in the FMR1 gene. In another embodiment, the extent of methylation is determined in the FREE2 portion of intron 1 of the FMR1 gene such as in a human subject including a human female subject with an FM or a developing FM. Examples of sites in FREE2 include CpG units 6/7 and 10-12.

The present disclosure teaches a method for identifying a methylation or other epigenetic profile in the genome of a cell indicative of a pathological condition associated with the FMR genetic locus, the method comprising screening for a change relative to the control in the extent of epigenetic modification of CpG and/or CpNpG sites located within:

(i) (a) FREE3 (I); (b) intron 2; and (c) an intron downstream of intron 2 or a homolog thereof or a portion or fragment thereof within the FMR1 gene;

(ii) two or more of (a) an intron; (b) an intron/exon boundary; (c) splicing region within the FMR genetic locus;

(iii) approximately one seventh or greater of an intron including an intron/exon boundary and/or a splicing region within the FMR genetic locus; and/or

(iv) the FREE2 portion of intron 1 of the FMR1 gene in a subject with an FM or a developing FM;

wherein a change in the extent of epigenetic modification is indicative of the presence of the pathological condition or a propensity to develop same. The nucleotide sequences of FREE3 (I) and intron 2 are set forth in SEQ ID NOs: 1 and 2, respectively and the present disclosure extends to their homologs and portions and parts thereof having at least 80% identity thereto or a nucleotide sequence capable of hybridizing to these sequences or their complementary forms under medium stringency conditions. Reference to FREE3 (I) and an intron such as intron 2 includes portions, fragments, parts, regions and domains thereof. The nucleotide sequence of intron 1 of the FMR1 gene containing all or a portion of FREE2(A), FREE2(B) and FREE2(C) is as set forth in SEQ ID NO:3. The present disclosure extends to homologs and portions and parts thereof having at least 80% identity to SEQ ID NO:3 or a nucleotide sequence capable of hybridizing to this sequence or its complementary form under medium stringency conditions.

In a particular embodiment, the epigenetic modification is methylation and RNA/DNA interactions.

The present disclosure further teaches a method for identifying a pathological condition in a subject associated with methylation within the FMR locus, the method comprising extracting genomic DNA from a cell of the subject and subjecting the DNA to an amplication reaction using primers selective of a region of the FMR genetic locus comprising CpG and/or CpNpG sites, the CpG and CpNpG sites located in

(i) a region in the FMR1 gene selected from:
(a) Fragile X-related Epigenetic Element 3 (I) [FREE3 (I)] comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;
(b) intron 2 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or fragment or portion thereof;

(ii) a region in the FMR genetic locus selected from: (a) two or more introns; an intron/exon boundary and/or splicing region; or (b) approximately one seventh or greater of an intron including an intron/exon boundary and/or a splicing region; and

(iii) one or more sites within the FREE2 portion of intron 1 of the FMR1 gene in combination with an FM;

and subjecting the DNA to a methylation assay to determine the extent of methylation of the DNA wherein a change in extent of methylation relative to a control is indicative of the presence or severity of the pathological condition or propensity to develop same. In an embodiment, the sites in FREE2 include CpG units 6/7 and 10-12.

Any methylation assay may be employed such as methylation sensitive PCR, methylation specific melting curve analysis (MS-MCA) or high resolution melting. (MS-HRM) [Dahl et al. (2007) supra; Wojdacz et al. (2007) Nucleic Acids Res. 35(6):e41]; quantification of CpG methylation by MALDI-TOF MS (Tost et al. (2003) Nucleic Acids Res 31(9):e50); methylation specific MLPA (Nygren et al. (2005) Nucleic Acids Res. 33(14):e128); methylated-DNA precipitation and methylation-sensitive restriction enzymes (COMPARE-MS) [Yegnasubramanian et al. (2006) Nucleic Acids Res. 34(3):e19] or methylation sensitive oligonucleotide microarray (Gitan et al. (2002) Genome Res. 12(1):158-164), as well as via antibodies. Other assays include NEXT generation (GEN) and DEEP sequencing or pyrosequencing.

Insofar as the methylation assay may involve an amplification, an amplification methodology may be employed. Amplification methodologies contemplated herein include the polymerase chain reaction (PCR) such as disclosed in U.S. Pat. Nos. 4,683,202 and 4,683,195; the ligase chain reaction (LCR) such as disclosed in European Patent Application No. EP-A-320 308 and gap filling LCR (GLCR) or variations thereof such as disclosed in International Patent Publication No. WO 90/01069, European Patent Application EP-A-439 182, British Patent No. GB 2,225,112A and International Patent Publication No. WO 93/00447. Other amplification techniques include Qβ replicase such as described in the literature; Stand Displacement Amplification (SDA) such as described in European Patent Application Nos. EP-A-497 272 and EP-A-500 224; Self-Sustained Sequence Replication (3SR) such as described in Fahy et al. (1991) PCR Methods Appl. 1(1):25-33) and Nucleic Acid Sequence-Based Amplification (NASBA) such as described in the literature.

A PCR amplification process is particularly useful in the practice of aspects enabled herein.

In an embodiment, prior to the PCR, either essentially all cytosines in the DNA sample are selectively de-aminated, but 5-methylcytosines remain essentially unchanged or essentially all 5-methylcytosines in the DNA sample are selectively de-aminated, but cytosines remain essentially unchanged. Cytosine-guanine (CpG) dinucleotides and CpNpG trinucleotides are detected, allowing conclusions about the methylation state of cytosines in the CpG dinucleotides and CpNpG trinucleotide in the DNA sample. This delamination is generally performed using a bisulfite reagent. After bisulfite treatment, the 5-methylcytosines residues are converted to thymine (T).

The sample DNA is only amplified by chosen PCR primers if a certain methylation state is present at a specific site in the sample DNA the sequence context of which is essentially complementary to one or more of the chosen PCR primers. This can be done using primers annealing selectively to bisulfite treated DNA which contains in a certain position either a TG or a CG or CNG, depending on the methylation status in the genomic DNA. Primers are designed based on particular regions around CpG and/or CpNpG sites or other FMR1 intronic regions. Introns or parts thereof including intron/exon boundaries and splicing regions downstream of intron 2 of FMR1 or downstream of the FMR1 gene itself are also contemplated herein as are (i) two or more of (a) an intron; (b) an intron/exon boundary; and/or (c) a splicing region; and/or (ii) approximately one seventh or greater of an intron including an intron/exon boundary and/or a splicing region within the FMR genetic locus; and/or (iii) a site within the FREE2 portion of the FMR1 gene in combination with an FM.

A technology which can alternatively be employed for methylation analysis utilizes base-specific cleavage followed by MALDI-TOF mass spectrometry on DNA after bisulfite treatment, where all the 5-methylcytosines residues are converted to thymine (T) or where all unmethylated cytosines residues are not converted to thymine (T). Primers are designed based on particular regions around CpG and/or CpNpG sites or other FMR1 intronic regions or downstream thereof. Primer sequences are designed to amplify without bias both converted and unconverted sequences using the PCR amplification process under the medium to high stringency conditions. The PCR products are in vitro transcribed and subjected to base specific cleavage and fragmentation analysis using MALDI-TOF MS. The size ratio of the cleaved products provides quantitative methylation estimates for CpG sites within a target region. The shift in mass for non-methylated (NM) from methylated (M) fragments for a single CpG site is −16 daltons due to the presence of an adenosine residue in the place of a guanosine. A software is then used to calculate methylation for each fragment based on this difference in mass, where the output methylation ratios are the intensities of methylated signal/[methylated+unmethylated signal]. If the fragment size overlaps for different CpGs, their methylation output ratio is calculated based on the sum of intensities for methylated/[methylated+unmethylated signal]. To distinguish how well the methylation output ratio for multiple fragments of a similar size represented methylation of separate CpG sites, for some amplicons both cytosine and thymidine cleave reactions can be performed (that produced fragments of different size) prior to fragment analysis. Silent peaks (S)—fragments of unknown origin, should not be taken into consideration if their size does not overlap with the fragments of interest. Methylation of CpG sites that have silent peaks (S) that overlap with the fragments of interest should be included in the analysis.

Hence, a method is provided for determining the methylation profile of one or more CpG or CpNpG sites located within the genome of a eukaryotic cell or group of cells, the method comprising obtaining a sample of genomic DNA from the cell or group of cells and subjecting the genomic DNA to primer-specific amplification within an intron of a genetic locus and assaying for extent of methylation relative to a control, including a change in the extent of methylation and associating this change with a pathological condition.

A “nucleic acid” as used herein, is a covalently linked sequence of nucleotides in which the 3′ position of the phosphorylated pentose of one nucleotide is joined by a phosphodiester group to the 5′ position of the pentose of the next nucleotide and in which the nucleotide residues are linked in specific sequence; i.e. a linear order of nucleotides. A “polynucleotide” as used herein, is a nucleic acid containing a sequence that is greater than about 100 nucleotides in length. An “oligonucleotide” as used herein, is a short polynucleotide or a portion of a polynucleotide. An oligonucleotide typically contains a sequence of about two to about one hundred bases. The word “oligo” may be used in place of the word “oligonucleotide”. The term “oligo” also includes a particularly useful primer length in the practice of the present disclosure of up to about 10 nucleotides.

As used herein, the term “primer” refers to an oligonucleotide or polynucleotide that is capable of hybridizing to another nucleic acid of interest under particular stringency conditions. A primer may occur naturally as in a purified restriction digest or be produced synthetically, by recombinant means or by PCR amplification. The terms “probe” and “primers” may be used interchangeably, although to the extent that an oligonucleotide is used in a PCR or other amplification reaction, the term is generally “primer”. The ability to hybridize is dependent in part on the degree of complementarity between the nucleotide sequence of the primer and complementary sequence on the target DNA.

The terms “complementary” or “complementarity” are used in reference to nucleic acids (i.e. a sequence of nucleotides) related by the well-known base-pairing rules that A pairs with T or U and C pairs with G. For example, the sequence 5′-A-G-T-3′ is complementary to the sequence 3′-T-C-A-5′ in DNA and 3′-U-C-A-5′ in RNA. Complementarity can be “partial” in which only some of the nucleotide bases are matched according to the base pairing rules. On the other hand, there may be “complete” or “total” complementarity between the nucleic acid strands when all of the bases are matched according to base-pairing rules. The degree of complementarity between nucleic acid strands has significant effects on the efficiency and strength of hybridization between nucleic acid strands as known well in the art. This is of particular importance in detection methods that depend upon binding between nucleic acids, such as those of the disclosure. The term “substantially complementary” is used to describe any primer that can hybridize to either or both strands of the target nucleic acid sequence under conditions of low stringency as described below or, preferably, in polymerase reaction buffer heated to 95° C. and then cooled to room temperature. As used herein, when the primer is referred to as partially or totally complementary to the target nucleic acid, that refers to the 3′-terminal region of the probe (i.e. within about 10 nucleotides of the 3′-terminal nucleotide position).

Reference herein to a stringency in relation to hybridization includes and encompasses from at least about 0 to at least about 15% v/v formamide and from at least about 1 M to at least about 2 M salt for hybridization, and at least about M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt′ for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex DNA decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C. Reference to at least “80% identity” includes 80, 81, 82, 83, 84, 85, 86, 87, 88, 89, 90, 91, 92, 93, 94, 95, 96, 97, 98, 99 and 100%.

The present disclosure enables determination of a methylation or other epigenetic profile of sites within an intron, intron/exon boundary and/or splicing region of a genetic locus in a genome of a eukaryotic cell or group of cells, the method comprising obtaining a sample of genomic DNA from the cell or group of cells, subjecting the digested DNA to an amplification reaction using primers selected to amplify a region of the genetic locus selected from:

(i) two or more of (a) an intron; (b) an intron/exon boundary; and/or (c) a splicing region;

(ii) approximately one seventh or greater of an intron including an intron/exon boundary and/or a splicing region; and/or

(iii) an intragenic site in combination with an expansion mutation;

and then subjecting the amplified DNA to methylation or other epigenetic detection means to determine relative to control the extent of methylation or other epigenetic modification wherein a change in epigenetic modification or other epigenetic modification relative to the control is indicative of a pathological condition associated with the genetic locus.

In an embodiment, enabled herein is a methylation profile of the sites within the FMR locus in a genome of a eukaryotic cell or group of cells, the methylation profile comprising the extent or level of methylation within the FMR locus, the method comprising obtaining a sample of genomic DNA from the cell or group of cells, subjecting the digested DNA to an amplification reaction using primers selected to amplify a region of the FMR genetic locus selected from:

(i) (a) FREE3 (ASFMR1 promoter) (I); (b) intron 2; (c) an intron, intron/exon boundary and/or splicing region downstream of intron 2 or part thereof and (d) the FREE2 portion of intron 1 of the FMR1 gene;

(ii) two or more of (a) an intron; (b) an intron/exon boundary; (c) a splicing region;

(iii) approximately one seventh or greater of an intron including an intron/exon boundary and/or a splicing region within the FMR genetic locus; and

(iv) site in the FREE2 portion of intron 1 of the FMR1 gene in, combination with an FM;

and then subjecting the amplified DNA to methylation detection means to determine relative to control the extent of methylation wherein a change in methylation relative to the control is indicative of a pathological condition associated with the FMR genetic locus.

The present disclosure further teaches a methylation profile of the sites within the FMR locus in a genome of a eukaryotic cell or group of cells, the methylation profile comprising the extent or level of methylation within the FMR locus, the method comprising obtaining a sample of genomic DNA from the cell or group of cells, subjecting the digested DNA to an amplification reaction using primers selected to amplify FREE 3(I) within the FMR1 gene and then subjecting the amplified DNA to methylation detection means to determine relative to control the extent of methylation wherein a change in methylation relative to the control is indicative of a pathological condition associated with the FMR genetic locus.

The present disclosure further enables a methylation profile of the sites within the FMR locus in a genome of a eukaryotic cell or group of cells, the methylation profile comprising the extent or level of methylation within the FMR locus, the method comprising obtaining a sample of genomic DNA from the cell or group of cells, subjecting the digested DNA to an amplification reaction using primers selected to amplify all or part of the FREE2 portion of intron 1 of the FMR1 gene and then subjecting the amplified DNA to methylation detection means to determine relative to control the extent of methylation wherein a change in methylation relative to the control is indicative of a pathological condition associated with the FMR genetic locus. This aspect further screening for the presence of an FM wherein the combination of hypermethylation in the FREE2 region and an FM is instructive as to cognitive impairment or a likelihood of developing same.

As indicated above, the cells may be a lymphoblast, a CVS cell, a blood cell, an amniocyte or an EBV transformed lymphoblast cell line. In addition, the methylation profile may be determined or one or both alleles a genetic locus and in selected cells where mosaicism has occurred. In particular, the extent of methylation can determine homozygosity or heterozygosity or mosaicism. Reference to “mosaicism” includes the situation wherein two or more populations of cells have different genotypes or epigenetic profiles at the genetic locus.

The diagnostic assay herein can also detect heterozygosity or mosaicism where the methylation pattern is indicative of, for example, in relation to an FMR genetic locus-associated pathology, an FM. The latter may also be conducted in combination with an assay to detect (CGG)ₙ expansion.

The present disclosure also teaches kits for determining the methylation or other epigenetic profile of one or more nucleotides at one or more sites within the genome of a eukaryotic cell or group of cells. The kits may comprise many different forms but in one embodiment, the kits comprise reagents for the bisulfite methylation assay.

A further embodiment of the present disclosure is a kit for the use in the above methods comprising primers to amplify an intron within a genetic locus.

In an embodiment, the present disclosure provides a use of primers which amplify regions of the FMR genetic locus, comprising CpG and/or CpNpG sites located within:

(i), the FMR1 gene selected from:
(a) Fragile X-related Epigenetic Element 3 [FREE3 (I)] comprising the nucleotide sequence set forth in SEQ ID NO:1 or a homolog thereof or portions or parts thereof defined by having at least 80% nucleotide sequence identity to SEQ ID NO:1 or which hybridizes to SEQ ID NO:1 or its complementary form under medium stringency conditions;
(b) intron 2 comprising the nucleotide sequence set forth in SEQ ID NO:2 or a homolog thereof or portions or parts thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or a portion or fragment thereof;

(ii) the FMR genetic locus selected from: (a) two or more introns; an intron/exon boundary; and/or a splicing region;

(iii) approximately one seventh or greater of an intron including an intron/exon boundary and/or a splicing region; and

(iv) a site in the FREE2 portion of intron 1 of the FMR1 gene in combination with an FM;

in the manufacture of a diagnostic kit or device to detect methylation of the FMR locus-associated with a pathological condition.

In relation to an embodiment, a kit is provided for the use in the above methods comprising primers identified by SEQ ID NOs:6 through 11 to amplify an intronic site within the FMR1 genetic locus. Please note that the nucleotide sequences in SEQ ID NOs:6 through 11 comprise primer and tag sequences. The present number extends to SEQ ID NO:6 through 11 as well as primer only portions therein. The primers may also include primers disclosed in PCT/AU2010/000169. Primers may also be used to exemplify FREE2 or part thereof or used to identify a subject with an FM.

The kit may also comprise instructions for use.

Conveniently, the kits are adapted to contain compartments for two or more of the above-listed components. Furthermore, buffers, nucleotides and/or enzymes may be combined into a single compartment.

As stated above, instructions optionally present in such kits instruct the user on how to use the components of the kit to perform the various methods of the present disclosure. It is contemplated that these instructions include a description of the detection methods of the subject disclosure, including detection by gel electrophoresis.

The present disclosure further enables kits which contain a primer for a nucleic acid target of interest with the primer being complementary to a predetermined nucleic acid target. In another embodiment, the kit contains multiple primers or probes, each of which contains a different base at an interrogation position or which is designed to interrogate different target DNA sequences. In a contemplated embodiment, multiple probes are provided for a set of nucleic acid target sequences that give rise to analytical results which are distinguishable for the various probes. The multiple probes may be in microarray format for ease of use.

A kit may comprise a vessel containing a purified and isolated enzyme whose activity is to release one or more nucleotides from the 3′ terminus of a hybridized nucleic acid probe and a vessel containing pyrophosphate. In one embodiment, these items are combined in a single vessel. It is contemplated that the enzyme is either in solution or provided as a solid (e.g. as a lyophilized powder); the same is true for the pyrophosphate. Preferably, the enzyme is provided in solution. Some contemplated kits contain labeled nucleic acid probes. Other contemplated kits further comprise vessels containing labels and vessels containing reagents for attaching the labels. Microtiter trays are particularly useful and these may comprise from two to 100,000 wells or from about six to about 10,000 wells or from about six to about 1,000 wells.

Another important application is in the high throughput screening of agents which are capable of demethylation genomes and in particular intronic regions within genomes. This may be important, for example, in de-differentiating cells and/or treating pathological conditions.

The present disclosure enables a method for screening for an agent which modulates methylation or other epigenetic modification of a genetic locus, the method comprising screening for a change relative to a control in the extent of methylation or other epigenetic modification in an intron, intron/exon boundary and/or splicing region and/or an intragenic region within the genetic locus which is associated with a pathological condition in the presences or absence of an agent to be tested, wherein an agent is selected if it induces a change in the extent of methylation or other epigenetic change. Agents include de-methylation agents and hyper-methylation agents, global and site specific.

In an embodiment, a method is provided for screening for an agent which modulates methylation of an FMR genetic locus in a mammalian cell including a human cell, the method comprising screening for a change relative to a control in the extent of methylation in a region selected from:

(i) Fragile X-related Epigenetic Element 3 [FREE3 (I)] in FMR1 comprising the nucleotide sequence set forth in SEQ ID NO:1 or a homolog thereof or portions or parts thereof defined by having at least 80% nucleotide sequence identity to SEQ ID NO:1 or which hybridizes to SEQ ID NO:1 or its complementary form under medium stringency conditions;

(ii) intron 2 of FMR1 comprising the nucleotide sequence set forth in SEQ ID NO:2 or a homolog thereof or portions or parts thereof defined by having at least 80% nucleotide sequence identity to SEQ ID. NO:2 or which hybridizes to SEQ ID NO:2 or its complementary form under medium stringency conditions;

(iii) an intron, intron/exon boundary and/or a splicing region downstream of intron 2 of FMR1 or a homolog thereof or a portion or fragment thereof;

(iv) two or more of (a) an intron; (b) an intron/exon boundary; (c) a splicing region within the FMR genetic locus;

(v) approximately one seventh or greater of an intron including an intron/exon boundary and/or a splicing region within the FMR genetic locus; and/or

(vi) a site with the FREE2 portion of intron 1;

in the presence or absence of an agent to be tested wherein the agent is selected if it induces a change in extent of methylation.

The present disclosure further teaches a method for monitoring the treatment of a genetic locus-associated disease including a nucleotide expansion disease in which the treatment modulates the methylation of the genetic locus, the method comprising monitoring for a change relative to a control or a pre and post-treatment sample in the extent of methylation within an intron, intron/exon boundary and/or splicing region of the genetic locus.

By “monitoring” includes diagnosis, prognosis, pharmacoresponsiveness, pharmacosensitivity, level of disease progression or remission, improving or declining health of a subject and the like.

As indicated above, conditions and disorders contemplated herein include a range of nucleotide expansion diseases such as but not limited to polyglutamine (polyQ) diseases such as Huntington's disease (HD), dentatorubropallid-oluysiantrophy (DRPLA), spinobulbar muscular atrophy or Kennedy disease (SBMA), spinocerebella ataxia Type 1 (SCA1), spinocerebella ataxia Type 2 (SCA2), spinocerebella ataxia Type 3 or Machado-Joseph disease (SCA3), spinocerebella ataxia Type 6 (SCA6), spinocerebella ataxia Type 7 (SCA7), spinocerebella ataxia Type 17 (SCA17) and non-polyQ diseases such as Fragile X syndrome (FXS), Fragile X-associated tremor or ataxia (FXTAS), Fragile XE mental retardation (FRAXE), Friedrich's ataxia (FRDA), FXPOI and other premutatin conditions, Fragile type, folic acid type, rare 12 (FRA12A), myotonic dystrophy (DM), spinocerebella ataxia (SCA8) and spinocerebella ataxias Type 12 (SCA12). Other conditions contemplated herein include autism (including co-morbid autism), mental retardation, Klinefelter's syndrome, RNA toxicity disease, Turner's syndrome, a modified X-chromosome, and cognitive impairment as well as learning and behavioral problems. Reference to a “modified” X-chromosome includes skewed X-inactivation, inversions, deletions, duplications, hybrids and any modification leading to X-chromosome inactivation.

Particular conditions include Fragile X-associated tremor or ataxia (FXTAS), Fragile XE mental retardation (FRAXE), Friedrich's ataxia (FRDA), premutation-related disorders such as but not limited to FXPOI, Fragile type, folic acid type, rare 12 (FRA12A), autism (including co-morbid autism), mental retardation, Klinefelter's syndrome, RNA toxicity disease, Turner's syndrome, a modified X-chromosome and cognitive impairment.

The present disclosure further teaches the identification of genes having introns with CpG or CpNpG sites or other methylation-sensitive restriction sites. The identification of these sites permits identification of potential regulatory regions which can be targeted for agonists or antagonists of gene expression.

In cases where the gene is methylated and silenced in affected individuals or tissues, compounds are screened in high throughput fashion in stable cell lines or individuals to identify drugs that result in demethylation and reactivation of the affected gene. Alternatively, a normal active copy of the affected gene is transfected as a transgene into cells to correct the defect. Such transgenes are introduced with modulating sequences that protect the transgene from methylation and keep it unmethylated and transcriptionally active.

In cases where the gene is unmethylated and transcriptionally active or transcriptionally over-active in affected individuals or tissues, compounds are screened in high throughput fashion in stable cell lines to identify drugs that result in methylation and silencing of the affected gene. Alternatively, a transgene encoding a double stranded RNA homologous to the affected sequences or homologs thereof, are transfected as a transgene into cells to methylate the gene, silence it and thereby correct the defect. Such double stranded RNA-encoding transgenes are introduced with modulating sequences which protect it from methylation, keep it transcriptionally active and producing double stranded RNA.

The present disclosure provides a computer program and hardware which monitors the changing state, if any, of extent of methylation over time or in response to therapeutic and/or behavioral modification. Such a computer program has important utility in monitoring disease progression, response to intervention and may guide modification of therapy or treatment. The computer program is also useful in understanding the association between increasing methylation and disease progression.

The computer program monitors in a quantitative or semi-quantitative manner one or more features including extent of methylation or other epigenetic modification in an intron of a genetic locus. In addition, the length of a nucleotide expansion may be determined or any epigenetic changes therein. In relation to a neuropathological condition, a behavioral assessment may be made using criteria associated with normal subjects or subjects considered to be suffering with a disease condition. For example, cognitive ability and/or behavioral deficiency can be measured as well as the general phenotype or clinical manifestations in subjects with a neurodevelopmental or neurodegenerative condition or other condition associated with nucleotide expansion.

Thus, in accordance with the present disclosure, values are assigned to the listed features which are stored in a machine-readable storage medium, which is capable of processing the data to provide an extent of disease progression or change in methylation or other epigenetic modification for a subject.

Thus, in a particular aspect, the disclosure teaches a computer program product for assessing progression of a pathological condition associated with the FMR locus in a subject, the product comprising:

(1) assigning a value to one or more of:
(a) change in of methylation or other epigenetic modification relative to a control in FREE3 (I) of FMR1 comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;
(b) change of methylation or other epigenetic modification relative to a control in intron 2 of FMR1 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;
(c) change of methylation in an intron, intron/exon boundary and/or splicing region downstream of intron 2 of FMR1 or a homolog thereof or a portion or fragment thereof;
(d) two or more of (i) an intron; (ii) an intron/exon boundary; (iii) a splicing region within the FMR genetic locus;
(e) approximately one seventh or greater of an intron including an intron/exon boundary and/or a splicing region within the FMR genetic locus;
(f) length of (CGG)ₙ expansion within the FMR genetic locus when considered in combination with (a) and/or (b);
(g) hypermethylation at one or more sites in a FREE2 portion on intron 1 of the FMR1 gene in combination with an FM;
(h) general phenotype or clinical manifestations in subjects with a neurodevelopmental or neurodegenerative condition;
(i) behavioral assessment criteria associated with normal subjects, PM subjects, GZ subjects and FM subjects;
(j) cognitive ability and/or behavioral deficiency;
(k) extent of transcription of genes within the FMR locus with the proviso that if any one of (d) through (f) is determined then one or more of (a) through (c) and/or (g) is also determined;

(2) means to converting the value to a code; and

(3) means to store the code in a computer readable medium and compare code to a knowledge database to determine whether the code corresponds to a pathological condition.

In a related aspect, the disclosure teaches to a computer for assessing an association between extent of methylation or other epigenetic modification within the FMR locus, the FMR locus and progression of a disease condition wherein the computer comprises:

(1) a machine-readable data storage medium comprising a data storage material encoded with machine-readable data, wherein the machine-readable data comprise index values associated with the features of one or more of:
(a) change in methylation or other epigenetic modification relative to a control in FREE3 (I) of FMR1 comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions upstream;
(b) change of methylation or other epigenetic modification relative to a control in CpG and/or CpNpG islands and island shores in intron 2 of the FMR1 gene comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;
(c) change in methylation of an intron, intron/exon boundary and/or splicing region downstream of intron 2 of FMR1 or a homolog thereof or a portion or fragment thereof;
(d) two or more (i) an intron; (ii) an intron/exon boundary; (iii) a splicing region within the FMR genetic locus;
(e) approximately one 7^{th} (seventh) or greater of an intron including an intron/exon boundary and/or a splicing region within the FMR genetic locus;
(f) length of (CGG)ₙ expansion within the FMR genetic locus when considered in combination with (a) and/or (b);
(g) hypermethylation at one or more sites in a FREE2 portion on intron 1 of the FMR1 gene in combination with an FM;
(h) general phenotype or clinical manifestations in subjects with a neurodevelopmental or neurodegenerative condition;
(i) behavioral assessment criteria associated with normal subjects, PM subjects, GZ subjects and FM subjects;
(j) cognitive ability and/or behavioral deficiency;
(k) extent of transcription of genes within the FMR locus with the proviso that if any one of (d) through (f) is determined then one or more of (a) through (c) and/or (g) is also determined;

(2) means to converting index value to a code; and

(3) means to store the code in a computer readable medium and compare code to a knowledge database to determine whether the code corresponds to a pathological condition.

The computer system of the present disclosure may also be linked to detection systems such as MALDI-TOF mass spectrometry machines.

The present disclosure further provides a web-based system where data on extent of methylation within a genetic locus (optionally together with clinical phenotype) are provided by a client server to a central processor which analyzes and compares to a control and optionally considers other information such as patient age, sex, weight and other medical conditions and then provides a report, such as, for example, a risk factor for disease severity or progression or status or response to treatment or an index of probability of a genetic locus-associated pathology in a subject.

Hence, knowledge-based computer software and hardware also form part of the present disclosure.

In an embodiment, the assays herein may be used in existing or newly developed knowledge-based architecture or platforms associated with pathology services. For example, results from the assays are transmitted via a communications network (e.g. the internet) to a processing system in which an algorithm is stored and used to generate a predicted posterior probability value which translates to the index of disease probability which is then forwarded to an end user in the form of a diagnostic or predictive report.

The assay may, therefore, be in the form of a kit or computer-based system which comprises the reagents necessary to detect the extent of methylation or other epigenetic modification within the genetic locus and includes computer hardware and/or software to facilitate determination and transmission of reports to a clinician.

The assay herein described permits integration into existing or newly developed pathology architecture or platform systems. For example, a method is provided of allowing a user to determine the status of a subject with respect to an FMR locus-associated pathology, the method including:

(a) receiving data in the form of extent of methylation or other epigenetic modification at a site within:

(A) the FMR1 gene selected from:
(i) Fragile X-related Epigenetic Element 3 (I) [FREE3 (I)] comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;
(ii) intron 2 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;
(iii) an intron, intron/exon boundary and/or splicing region downstream of intron 2 or a homolog thereof or a fragment or portion thereof; and
(iv) the FREE2 portion of intron 1 of the FMR1 gene in combination with an FM;

(B) the FMR genetic locus selected from:
(i) two or more of (a) an intron; (b) an intron/exon boundary; (c) a splicing region;
(ii) approximately one seventh or greater of an intron including an intron/exon boundary and/or a splicing region; and
(iii) an intragenic region in combination with a CGG expansion mutation;
   wherein the extent of methylation or other epigenetic modification provides a correlation to the presence, state, classification or progression of the pathology; by transferring the data from the user via a communications network;

(c) processing the subject data via multivariate or univariate analysis to provide a disease index value;

(d) determining the status of the subject in accordance with the results of the disease index value in comparison with predetermined values; and

(e) transferring an indication of the status of the subject to the user via the communications network. Reference to the multivariate or univariate analysis includes an algorithm which performs the multivariate or univariate analysis function.

Conveniently, the method generally further includes:

(a) having the user determine the data using a remote end station; and

(b) transferring the data from the end station to the base station via the communications network.

The base station can include first and second processing systems, in which case the method can include:

(a) transferring the data to the first processing system;

(b) transferring the data to the second processing system; and

(c) causing the first processing system to perform the multivariate analysis function to generate the disease index value.

The method may also include:

(a) transferring the results of the multivariate or univariate analysis function to the first processing system; and

(b) causing the first processing system to determine the status of the subject.

In this case, the method also includes at least one of:

(a) transferring the data between the communications network and the first processing system through a first firewall; and

(b) transferring the data between the first and the second processing systems through a second firewall.

The second processing system may be coupled to a database adapted to store predetermined data and/or the multivariate analysis and/or univariate analysis function, the method including:

(a) querying the database to obtain at least selected predetermined data or access to the multivariate or univariate analysis function from the database; and

(b) comparing the selected predetermined data to the subject data or generating a predicted probability index.

The second processing system can be coupled to a database, the method including storing the data in the database.

The method can also include having the user determine the data using a secure array, the secure array of elements capable of determining the extent of methylation in an intron with a genetic locus and having a number of features each located at respective position(s) on the respective code. In this case, the method typically includes causing the base station to:

(a) determine the code from the data;

(b) determine a layout indicating the position of each feature on the array; and

(c) determine the parameter values in accordance with the determined layout, and the data.

The method can also include causing the base station to:

(a) determine payment information, the payment information representing the provision of payment by the user; and

(b) perform the comparison in response to the determination of the payment information.

The present disclosure also teaches a base station for determining the status of a subject with respect to a pathology associated with a genetic locus such as the FMR locus, the base station including:

(a) a store method;

(b) a processing system, the processing system being adapted to;

(c) receive subject data from the user via a communications network, the data; including extent of methylation within the genetic locus wherein the level or methylation or epigenetic modification relative to a control provides a correlation to the presence, state, classification or progression of the pathology;

(d) performing an algorithmic function including comparing the data to predetermined data;

(e) determining the status of the subject in accordance with the results of the algorithmic function including the comparison; and

(f) output an indication of the status of the subject to the user via the communications network.

The processing system can be adapted to receive data from a remote end station adapted to determine the data.

The processing system may include:

(a) a first processing system adapted to:
(i) receive the data; and
(ii) determine the status of the subject in accordance with the results of the multivariate or univariate analysis function including comparing the data; and

(b) a second processing system adapted to:
(i) receive the data from the processing system;
(ii) perform the multivariate or univariate analysis function including the comparison; and
(iii) transfer the results to the first processing system.

The determination of the extent of methylation or other epigenetic modification within the FMR locus at a site within the FMR1 gene selected from:

(i) Fragile X-related Epigenetic Element 3 (I) [FREE3 (I)] comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;

(ii) intron 2 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;

(iii) an intron, intron/exon boundary and/or splicing region downstream of intron 2 or a homolog thereof or a portion or fragment thereof;

(iv) two or more of (a) an intron; (b) an intron/exon boundary; (c) a splicing region; or

(v) approximately one seventh or greater of an intron including an intron/exon boundary and/or a splicing region within the FMR genetic locus; and

(vi) the FREE2 portion of intron 1;

enables establishment of a diagnostic or prognostic rule based on the extent of methylation relative to controls. Alternatively, the diagnostic or prognostic rule is based on the application of a statistical and machine learning algorithm. Such an algorithm uses relationships between methylation profiles and disease status observed in training data (with known disease status) to infer relationships which are then used to predict the status of patients with unknown status. An algorithm is employed which provides an index of probability that a patient has an FMR locus-associated pathology. The algorithm performs a multivariate or univariate analysis function.

Hence, the present disclosure teaches a diagnostic rule based on the application of statistical and machine learning algorithms. Such an algorithm uses the relationships between epigenetic profile and disease status observed in training data (with known disease status) to infer relationships which are then used to predict the status of patients with unknown status. Practitioners skilled in the art of data analysis recognize that many different forms of inferring relationships in the training data may be used without materially changing the present disclosure.

The present disclosure contemplates the use of a knowledge base of training data comprising extent of methylation within a genetic locus such as the FMR genetic locus from a subject with locus-associated pathology to generate an algorithm which, upon input of a second knowledge base of data comprising levels of the same biomarkers from a patient with an unknown pathology, provides an index of probability that predicts the nature of unknown pathology or response to treatment.

The term “training data” includes knowledge of the extent of methylation relative to a control. A “control” includes a comparison to levels in a healthy subject devoid of a pathology or is cured of the condition or may be a statistically determined level based on trials.

The present disclosure contemplates, therefore, the use of the methylation, including epigenetic profile of intronic sites within the FMR genetic locus and in particular the FMR1 gene to assess or determine the status of a subject with respect to disease, to stratify a subject relative to normal controls or unhealthy subjects, to provide a prognosis of recovery or deterioration and/or to determine the pharmacoresponsiveness or pharmacosensitivity of a subject to treatment or an agent for use in treatment and/or determine applicability for other treatment options including behavioral intervention, and the like. By “intronic sites” includes intron/exon boundaries and splicing regions.

Hence, another aspect of the present disclosure provides a method of allowing a user to determine the status, prognosis and/or treatment response of a subject with respect to an FMR locus-associated pathology, the method including:

(a) receiving data in the form of extent of methylation or other epigenetic modification at a site in the FMR locus selected from:
(i) Fragile X-related Epigenetic Element 3 [FREE3 (I)] comprising the nucleotide sequence set forth in SEQ ID NO:1 or a homolog thereof or portion or part thereof defined by having at least 80% nucleotide sequence identity to SEQ ID NO:1 or which hybridizes to SEQ ID NO:1 or its complementary form under medium stringency conditions;
(ii) intron 2 comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;
(iii) an intron, intron/exon boundary and/or splicing region downstream of intron 2 or a homolog or a portion or fragment thereof;
(iv) two or more of (a) an intron; (b) an intron/exon boundary; (c) a splicing region within the FMR locus;
(v) approximately one seventh or greater of an intron including an intron/exon boundary and/or a splicing region within the FMR genetic locus; and
(vi) the FREE2 portion of intron 1;
   wherein the extent of methylation or epigenetic modification provides a correlation to the presence, state, classification or, progression of the pathology;

(b) transferring the data from the user via a communications network;

(c) processing the subject data via multivariate or univariate analysis to provide a disease index value;

(d) determining the status of the subject in accordance with the results of the disease index value in comparison with predetermined values; and

(e) transferring an indication of the status of the subject to the user via the communications network.

Aspects disclosed herein are further described by the following non-limiting Example. In these Examples, materials and methods as outlined below were employed.

### Patient Samples

FXS, premutation and healthy control EBV transformed lymphoblast cell lines were obtained from the tissue culture storage repository of the Murdoch Childrens Research Institute, Melbourne, Victoria, Australia or purchased from Coriell.

### DNA Extraction

DNA for CGG repeat size PCR and methylation analysis was obtained either from 200 μl venous blood samples anti-coagulated with EDTA or from EBV transformed lymphoblasts 1 to 5×10⁶ cells per sample and extracted using a BIO ROBOT M48 DNA Extractor, as per manufacturer's instructions (Qiagen Inc., Hilden, Germany). DNA for Southern blot or methylation analysis was extracted from 3 ml blood samples anti-coagulated with EDTA or from EBV transformed lymphoblasts 5 to 10×10⁶ cells per sample.

### CGG Repeat Size PCR Amplification

CGG repeat size for all samples was initially assessed using a fully validated PCR assay with precision of +/− one triplet repeat across the normal and GZ ranges, performed using a fragment analyzer (MegaBace, GE Healthcare), with the higher detection limit of 170 repeats, as previously described (Khaniani et al., Mol Cytogenet 1(1):5, 2008). Briefly, PCR amplifications were performed using primers:

[SEQ ID NO: 32]
r(5'- GCTCAGCTCCGTTTCGGTTTCACTTCCGGT-3);
and
[SEQ ID NO: 33]
f(5'AGCCCCGCACTTCCACCACCAGCTCCTCCA-3'),

(Fu et al., Cell 67(6):1047-1058, 1991) in a total volume of 25 μl containing 50 ng of genomic DNA, 0.75 pmol of each primer, 8 μl of 5×Q-Solution (Qiagen Inc., Hilden, Germany), 2.5 μl of 10×PCR Buffer and 1 unit of HotStarTaq Plus DNA polymerase (Qiagen Inc., Hilden, Germany) in a Gene Amp@ PCR System 9700. The PCR cycling profile was as follows: initial denaturation at 98° C. for 5 minutes; 35 cycles at 98° C. for 45 seconds, 70° C. for 45 seconds, and 72° C. for 2 minutes, and a final extension at 72° C. for 10 minutes. Alleles were sized by capillary electrophoresis using an automatic sequencer (MegaBACETM 1000—GE HealthCare Amershm) with size standards (HealthCare) and controls of lengths 10, 23, 29, 30, 52 and 74 repeats determined by sequencing in-house or obtained from Coriel Cell Repositories web site (http://www.phppo.cdc.gov/dls/genetics/qcmaterials/).

### CGG Repeat Size by Southern Blot

CGG sizes were assessed using a fully validated Southern Blot procedure with appropriate normal and abnormal controls for samples where the products could not be amplified using. PCR (Fu et al 1991, supra; Francis et al., Mol Diagn 5(3):221-225, 2000). Briefly, 5 mg of DNA was digested with Pst1 (Boehringer Mannheim, Castle Hill, Australia), separated on 1% w/v agarose gels, and analyzed by Southern blot hybridization. The FMR-1 gene was detected using Southern blot analysis with probe Fxa3 and an X chromosome control probe, pS8 (Yu et al., Science 252(5010):1179-1181, 1991). Probes were labeled using random oligonucleotide priming (Boehringer, Mannheim) with [a32-P]CTP (NEN Dupont, Boston, Mass.). Autoradiography was performed at −80° C., with intensifying screens and Kodak XAR films (Sigma-Aldrich).

### Methylation Sensitive Southern Blot Analysis

Methylation of the classical FMR1 CpG island was assessed using a fully validated methyl sensitive Southern Blot procedure with appropriate normal and abnormal controls, as previously described (Tassone et al., J. Mol. Diagn 10:43-49, 2008). Briefly, EcoRI and NruI digestion was performed on 7 to 9 μg of DNA, and separated on a 0.8% w/v agarose/Tris acetate EDTA (TAE) gel. The DNA was denatured with HCL and NaOH, transferred to a charged nylon membrane and analyzed by Southern blot hybridization. The FMR1 alleles were detected using Southern blot analysis with probe StB12.3, labeled with Dig-11-dUTP by PCR (PCR Dig Synthesis kit; Roche Diagnostics). Autoradiography was performed with intensifying screens and Fuji Medical X-Ray film (Bedford, UK) and FMR1 methylation values for the expanded alleles were calculated as preciously described (Tassone et al., 2008 supra). The FMR1 activation ratios for female samples were calculated based on the following formula: optically scanned density of the 2.8 kb band/combined densities of the 2.8 kb and 5.2 kb bands (where the 2.8 kb band represents the proportion of normal active X and the 5.2 kb band represents the proportion of normal inactive X), as preciously described (de Vries et al., 1996 supra).

### MALDI-TOF Methylation Analysis

### Bisulfite Treatment

Bisulfite treatment of genomic DNA at 0.5 μg per sample was performed using XCEED kit from MethylEasy (Human Genetic Signatures, Sydney, Australia) for sample sets of n<40. For sample sets n>40 96 well Methylamp kit from Epigentek (Brookly, N.Y., USA) was used. Duplicate bisulfite reactions were made from each sample, and six of the same control DNA samples spiked with DNA from an FXS patient cell line at 0, 33.3, 50, 66.6 or 100% were included as standards within each run, as an indicator of the inter-run variation in the degree of bisulfite related bias. Protocols were performed according to the manufacturer's instructions. Briefly, for the MethylEasy conversion, 20 μl of genomic DNA (0.5 μg total) was mixed with 2.2 μl of 3 μl NaOH, and incubated at 37° C. for 15 minutes, then denatured by 45 minute incubation at 80° C. 240 μl of the reagent #3 (XCEED kit, Human Genetic Signatures. Sydney, Australia) were then added to the mixture, which was transferred into the purification column and spun down at 10,000 g for 1 minute. The captured DNA was then washed in Reagent #4 (XCEED kit, Human Genetic Signatures, Sydney, Australia), and DNA eluted twice by placing 50 μl of the pre-warmed solution #5 (XCEED kit, Human Genetic Signatures, Sydney, Australia) onto column membrane, which was incubated for 1 minute at room temperature, and spun down at 10,000 g for 1 minute. The eluted DNA was then incubated at 95° C. for 20 minutes, with resulting final concentration at ˜20 ng/μl per sample.

For the Methylamp conversion, 7 μl of genomic DNA (0.5 μg total) was mixed with 5 μl of the CF3 (Methylamp kit, Epigentek, Brookly, N.Y., USA) solution diluted 1:10 in distilled water, in each well of the 96 well plate. The DNA was denatured by placing the plate at 65° C. for 90 minutes. It was then captured in the filter plate and washed in 150 μl of the CF5 solution (Methylamp kit, Epigentek, Brookly, N.Y., USA), then twice in 250 μl of 80% v/v ethanol. The filter plate was then incubated in the CF3/90% v/v ethanol solution, and washed twice in 90% v/v ethanol, as per manufacturer's instructions. The modified and cleaned DNA was then eluted with 40 μl of the CF6 solution (Methylamp kit, Epigentek, Brookly, N.Y., USA), with resulting converted DNA final concentration at ˜20 ng/μl per sample. For the short term storage the converted DNA was kept at −20° C., and for storage of more than 3 months it was kept at −80° C.

### PCR and In Vitro Transcription

The primers used to amplify the target regions and the annealing temperatures are listed in Tables 3 and 4. Each bisulfite converted sample was analyzed in duplicate PCR reactions, carried out in a total volume of 5 μl using 1 pmol of each primer, 40 μM dNTP, 0.2 U Hot Star Taq DNA polymerase (Qiagen Inc., Hilden, Germany), 1.5 mM MgCl₂ and buffer supplied with the enzyme (final concentration 1×). The reaction mix was pre-activated for 15 min at 95° C., followed by 45 cycles of amplification at 94° C. for 20 s, primer specific annealing for 30 s and 72° C. for 1 min followed by 72° C. for 3 min. The PCR products were run on 1.5% w/v agarose gel to confirm successful PCR amplification and efficiency. The DNA was then cleaned up and the T or C-cleavage reactions were carried out (T-cleave for Amplicons 1 to 5, C-cleave for Amplicon 5 only) as per manufacturer's instructions (SEQUENOM, San Diego, Calif.). Briefly, unincorporated dNTPs were dephosphorylated by adding 1.7 μl H₂O and 0.3 U Shrimp Alkaline Phosphatase (SAP) [SEQUENOM, San Diego] to PCR products, which were incubated at 37° C. for 20 min, and 10 min at 85° C. to heat-inactivate the SAP. The transcription was performed on 2 μl of template DNA in the 6.5 ul reaction consisting of 20 U of the T7 R&DNA polymerase (Epicentre, Madison, Wis.) to incorporate either dCTP or dTTP; Ribonucleotides at 1 nM and the dNTP substrate at 2.5 mM, with other components used as recommended (SEQUENOM, San Diego). RNase A (SEQUENOM, San Diego) was then added to the mix to cleave the in vitro transcript. The mix was diluted to 27 μl in H₂O, and 6 mg CLEAN Resin (SEQUENOM, San Diego, Calif.) was added for conditioning of the phosphate backbone prior to MALDI-TOF MS. The SEQUENOM Nanodispenser was then used to spot the samples onto a SpectroCHIP for subsequent analysis. MassARRAY mass spectrometer (Bruker-SEQUENOM) was then used to collect mass spectra, which were analyzed using the EpiTYPER software (Bruker-SEQUENOM). The calculation of the output methylation ratios for each CpG unit were based on the ratio of the signal intensities for the fragment from a methylated CpG unit/[methylated+unmethylated CpG units]. Further details are described in (Godler et at, 2010 supra).

### RNA Extractions and Quality Assessments

Total RNA was extracted and purified using the Rneasy extraction kit, as per manufacturer's instructions (Qiagen Inc., Hilden, Germany). RNA concentrations were measured in triplicate using a NanoDrop ND-1000 Spectrophotometer, with purity being determined by the A260/A280 ratio using the expected values between 1.8 and 2. Total RNA quality and the degree of DNA contamination was also assessed using capillary electrophoresis Standard Sens Kit (Bio-rad), which involved descriptive comparison of chromatographic features based on previous publications using this system (Fleige and Pfaffl (2006) Mol Aspects Med 27(2-3):126-139). Each RNA sample was then diluted to 30 ng/ul, to be used in for reverse transcription real-time PCR analysis, where mRNA quality at the Xq27.3 region was initially assessed by examining the relationship between 5′ and 3′ levels of FMR1 mRNA.

### Standard Reverse Transcription Real-Time PCR

Reverse transcription was performed one reaction per sample using the Multiscribe Reverse Transcription System, 50 units/μl (Applied Biosystems). The 7900HT Fast Real Time PCR (Applied Biosystems) was used to quantify FMR1-5′, FMR1-3′, ASFMR1 (−1), (−2), (−3), GAPDH, B2M, and GUS, using the relative standard curve method. The target gene and the internal control gene dynamic linear ranges were performed on a series of doubling dilutions of an RNA standard (160-4 ng/μl). Since, both ASFMR1 assays do not target an exon/exon boundary, to minimize the impact of potential DNA contamination on the expression results, a no reverse transcription enzyme control was included for every sample. The difference between the plus and minus no reverse transcriptase control was considered as the ASFMR1 expression value for each sample. Previously published sequences were be used for primers and probe for: FMR1-5′ and GUS (32); FMR1-3′ (41). The following ASFMR1 primers and probes were designed using Primer Express 3.0 (Applied Biosystems):

ASFMR1 (-1) - Fw Primer
[SEQ ID NO: 34]
(CCGCGGAATCCCAGAGA);
Rv Primer:
[SEQ ID NO: 35]
(CAGTGGCGTGGGAAATCAA);
Probe:
[SEQ ID NO: 36]
(FAM-TGGGATAACCGGATGCA-MGB).
ASFMR1 (-2) - Fw Primer:
[SEQ ID NO: 37]
(ACACCCTGTGCCCTTTAAGG);
Rv Primer:
[SEQ ID NO: 38]
(TCAAAGCTGGGTCTGAGGAAAG);
Probe:
[SEQ ID NO: 39]
(VIC-TCGGGATCTCAAAATGT-TAMRA).
ASFMR1 (-3) - Fw Primer:
[SEQ ID NO: 40]
(CCCCAGAATGAGAGGATGTTG);
Rv Primer:
[SEQ ID NO: 41]
(GCCCTAGATCCACCGCTTTAA);
Probe:
[SEQ ID NO: 42]
(FAM-TGCTGGTGGAACTC-MGB).

FMR1-5′, FMR1-3′, ASFMR1 primers and probes were be used at concentrations of 18 μM and 2 μM, respectively. GAPDH and B₂M primer/probe mixes will be obtained from PrimerDesign (PerfectProbe ge-PP-12-hu kit) and used at concentration of 2 μM. All of the above assays were single-plexed, with each sample assayed in duplicate 10 μl PCR reactions. The reactions consisted of 5.8 mM MgCl2, 1 μl Buffer A (Applied Biosystems), 3.35 μl Rnase-free water, 1.2 mM dNTPs, 0.01 units/μl of AmpliTaq Gold, 0.5 μl of TaqMan probe and 0.5 μl forward and reverse primers, and 1 μl of the reverse transcription (cDNA) reaction. The annealing temperature for thermal cycling protocol was 60° C. for 40 cycles. The samples were quantified in arbitrary units (au) in relation to the standard curves performed on each plate, standardized to the mean of the 3 internal control genes (GUS, GAPDH and B₂M).

### Amplicons

Amplicons were amplified using the primers and conditions shown in Table 3. Table 4 shows prominent regulatory motif locations inclusive and proximal to FREE2 in FREE3. Amplicon 5 is as described by Godler et al. (2010) Hum Mol Genet, [Epub ahead of print] doi: 10.1093/hmg/ddq1037, the contents of which are incorporated by reference.

TABLE 3
Amplicon details used for MALDI-TOF methylation analysis of the regions
greater than 0.2kb 3' of the CGG expansion at the Xq27.3 locus

| | | | Distance | | |
|---|---|---|---|---|---|
| Amplicon | | Size | 3' of CGG | Primer sequence(in capitals) | SEQ |
| No. | Annealing Temperature | (kb) | (kb): | and tag (in lower case) | ID NO: |
| FREE2(B) | (I) 94° C. 4 min; 25 | 500 | 0.207 | Fw: 5′- | 11 |
| | cycles of: touchdown | | | aggaagagagGGTTTTTTT | |
| | PCR −0.5° C. per cycle | | | GAAATTTTTGGATTTA-3′ | |
| | −94° C. 20 s; 64° C. 30 s; | | | (SEQ ID NO: 6] | |
| | 72° C. 1 min. | | | Rv: 5′- | 12 |
| | (II) 20 cycles of: 94° C. | | | cagtaatacgactcactataggga | 9 |
| | for 20 s, 59° C. for 30 s, | | | gaaggctTAAAACCTATTAA | (genomic |
| | 72° C. for 1 min. | | | AAACCCCTCTCC-3′ (SEQ ID NO: 7) | sequence) |
| | (III) 72° C. for 3 min; | | | | |
| | 4° C. forever | | | | |
| FREE2(C) | (I) 94° C. 4 min; 25 | 302 | 0.504 | Fw: 5′- | 13 |
| | cycles of: touchdown | | | aggaagagagTAAGAGGGTTTTA | |
| | PCR −0.5° C. per cycle | | | GGTTTTTTTTGG-3′ (SEQ ID NO: 8) | |
| | −94° C. 20 s; 64° C. 30 s; | | | Rv: 5′- | 14 |
| | 72° C. 1 min. | | | cagtaatacgactcactatagggagaa | 10 |
| | (II) 20 cycles of: 94° C. | | | ggctAAAACATATACATTCCTAA | (genomic |
| | for 20 s, 59° C. for 30 s, | | | ATTTACCCC-3′ (SEQ ID NO:9) | sequence) |
| | 72° C. for 1 min. | | | | |
| | (III) 72° C. for 3 min; | | | | |
| | 4° C. forever | | | | |
| FREE3 | (I) 94° C. 4 min; 25 | 327 | 9.739 | Fw: 5′- | 15 |
| | cycles of: touchdown | | | aggaagagagTTTTTTTTATATAG | |
| | PCR −0.5° C. per cycle | | | GTATTTGTAAAGGATG-3′ | |
| | −94° C. 20 s; 64° C. 30 s; | | | (SEQ ID NO: 10) | |
| | 72° C. 1 min. | | | Rv: 5′- | 16 |
| | (II) 20 cycles of: 94° C. | | | cagtaatacgactcactatagggagaagg | 11 |
| | for 20 s, 59° C. for 30 s, | | | ctTCTCTAATTTCTTTCTTC | (genomic |
| | 72° C. for 1 min. | | | ACATTCAAAA-3′ | sequence) |
| | (III) 72° C. for 3 min; | | | (SEQ ID NO: 11) | |
| | 4° C. forever | | | | |

TABLE 4
Prominent regulatory motif locations inclusive and
proximal to FREE2 and FREE3 regions

| Transcription | | | | | | |
|---|---|---|---|---|---|---|
| factor sites/ | | | | | | |
| potential | | | Sequence | | | SEQ |
| regulatory | Sequence on the | | homology | Amplicon | | ID |
| motifs | sense strand | Strand | (%) | # | CpG unit location | NO: |
| GATA-1 | GGCGATGGCT | LEADING | 95 | FREE2(A) | CpG15 and CpG16 | 12 |
| HSF2 | TGAATATTCG | LEADING | 96 | FREE2(B) | CpG7 and CpG8/9 | 13 |
| C/EBP | AAGTTTCCAAAGA | LAGGING | 95 | N/A | 3′ of FREE2(B) CpG10 | 14 |
| CdxA | TATTATTATT | LAGGING | 99 | N/A | 3′ of FREE2(B) CpG10 | 15 |
| AML-1a | ACCACA | LAGGING | 100 | N/A | 3′ of FREE2(B) CpG10 | 16 |
| AML-1a | TGTGGTG | LEADING | 100 | N/A | 3′ of FREE2(B) CpG10 | 17 |
| CdxA | TATAAAT | LAGGING | 100 | N/A | 3′ of FREE2(B) CpG10 | 18 |
| CdxA | TATAAAT | LAGGING | 100 | N/A | 3′ of FREE2(B) CpG10 | 19 |
| CdxA | AATAATAT | LEADING | 99 | N/A | 3′ of FREE2(B) CpG10 | 20 |
| HFH-1/HFH-2 | AAATAAACAAT | LAGGING | 97 | N/A | 3′ of FREE2(B) CpG10 | 21 |
| CdxA | CATAAAT | LAGGING | 100 | N/A | 3′ of FREE2(B) CpG10 | 22 |
| SRY | TTTGTTT | LAGGING | 100 | N/A | 3′ of FREE2(B) CpG10 | 23 |
| SRY | TTTGTTT | LAGGING | 100 | N/A | 3′ of FREE2(B) CpG10 | 24 |
| SRY | TTGTTTA | LAGGING | 99 | N/A | 3′ of FREE2(B) CpG10 | 25 |
| S8 | TTTATTTAATTAAGTT | LEADING | 96 | N/A | 3′ of FREE2(B) CpG10 | 26 |
| SRY | AAACAAA | LEADING | 100 | 5′ of | 5′ of FREE3 CpG1 | 27 |
| | | | | FREE3 | | |
| CdxA | TATAATT | LEADING | 99 | FREE3 | CpG1 | 28 |
| Oct-1 | TTTATGCTAATT | LEADING | 99 | FREE3 | Between CpG1 and CpG2 | 29 |

The following materials and methods relate specifically to methylation studies of intron 1 of the FMR1 gene and epigenetic association with cognitive impairment set forth in Example 8.

### Subjects

Participants comprised 50 PM and 20 FM carrier human females and 21 control females. Other participants included 14 PM and 2 FM carrier human females. The standardized assessments of cognitive status for PM and FM participants were performed using Wechsler Adult Intelligence Scale (WAIS-III) IQ tests. The PM carriers were between 26 and 67 years of age. The FM carriers were between 7 and 35 years of age. The controls were between 2 and 38 years of age. Fifteen controls had no clinical history of developmental delay. All controls had CGG allele sizes below 40 repeats.

### Molecular Studies

Three to 10 ml of blood was collected into EDTA tubes for molecular analyses from the participants at the time of phenotypic testing. DNA was extracted as described using manual and automated extraction methods (Godler et al. (2010) Hum Mol Genet 19(8):1618-1632; Khaniani et al. (2008) supra; Loesch et al. (2003) Neuropsychology 17(4):646-657; Tassone et al. (2008) supra).

A fully validated PCR assay was initially used to determine CGG repeat sizes using a fragment analyzer (MegaBace, GE Healthcare), with a higher detection limit of 170 repeats, as described (Khaniani et al. (2008) supra). For samples with CGG repeat sizes within the PM and FM range methylation of the FMR1 CpG island was assessed using a fully validated methyl sensitive Southern blot procedure with appropriate normal and abnormal controls, as described (Tassone et al. (2008) supra; de Vries et al. (1996) supra); EcoRI and NruI digestion was performed on 7 to 9 μg of DNA. Separation of fragments was performed on a 0.8% w/v agarose/Tris acetate EDTA (TAE) gel; DNA was denatured with HCl and NaOH, transferred to a charged nylon membrane. Southern blot analysis used probe StB12.3, labeled with Dig-11-dUTP by PCR (PCR Dig Synthesis kit; Roche Diagnostics) to detect the FMR1 alleles. The FMR1 activation ratios for female samples was calculated based on the ratio of density (optically scanned) of the 2.8 kb band to combined densities of the 2.8 kb and 5.2 kb bands, where the 2.8 kb band represents the proportion of normal active X and the 5.2 kb band represents the proportion of normal inactive (methylated) X (de Vries et al. (1996) supra).

Blood smears for FM carriers were made within 24 hours of blood collection, and FMRP immunoreactivity assessment was conducted on as described (Tassone et al. (1999) Am J Med Genet 84(3):250-261; Willemsen et al. (1995) Lancet 345(8958):1147-1148; Loesch et al. (2002) Am J Med Genet 107(2):136-142). FMRP expression was presented as the percentage of lymphocytes that were positive in staining for FMRP.

Sequenom methylation evaluation tools were used to determine methylation output ratio measurements for FREE2 sites (within exon1 and intron 1) as described (Godler et al. (2010) supra). Briefly, bisulfite treatment was performed on 0.5 ug of genomic DNA at 0.5 ug per sample using XCEED kit from MethylEasy (Human Genetic Signatures, Sydney, Australia). PCR and in vitro transcription of FREE2 was performed as described (Godler et al. (2010) supra). The Squenom Nanodispenser was used to spot the samples onto a SpectroCHIP for subsequent analysis. MassARRAY mass spectrometer (Bruker-Sequenom) was then used to collect mass spectra, which were analyzed using the EpiTYPER 1.0.5 software (Bruker-Sequenom). The calculation of the output methylation ratios for each CpG unit was based on the ratio of the signal intensities for the fragment from a methylated CpG unit/[methylated+unmethylated CpG units] as described (Godler et al. (2010) supra).

Each sample was analyzed in duplicate and would result in two separate methylation output ratios reflecting technical variation resulting from bisulfite conversion, PCR and mass cleave reactions. Only samples that had the methylation output ratio within 35% of the mean of the two technical duplicates were utilized for further analysis. For the assay sensitivity and specificity assessments, methylation positive thresholds were determined for each CpG unit, which were methylation output ratios that would be optimal in distinguishing the low functioning (IQ<70) from high functioning (IQ>70) individuals. This threshold for CpG6/7 was 0.47, while for CpG10-12 it was 0.37.

### Statistical Analysis

The relationships between methylation values and cognitive outcome were assessed using a significance test in a simple linear regression. The analyses were conducted using the publicly available R statistical computing package (R Development Core Team 2007 R: A Language and Environment for Statistical Computing, R Foundation for Statistical Computing, Vienna, Austria, ISBN 3-900051-07-0. URL: http://www.r-project.org/.

### Example 1

### Mapping Methylation of the FMR Genetic Locus Using High Throughput Mass Spectrometry

The structure of the FMR genetic locus is shown in FIG. 1A and comprises the FMR1 promoter, and FMR1 and ASFMR1 genes. A CGG repeat is located within the 5′ (UTR) of the FMR1 gene. ASFMR1 spans the CGG expansion in the antisense direction and is also regulated by another promoter located in the exon 2 of FMR1. The FREE2 located downstream of the CGG expansion. The FREE3 region is located within intron 2 of FMR1 downstream of the second ASFMR1 promoter.

The primers utilized for MALDI-TOF methylation analysis targeted 6 regions at the Xq27.3 locus designated as FREE2(A) [described as amplicon 5 in Godler et al., 2010 supra]; FREE2(B); FREE2(C); FREE2(D); FREE2(E); FREE2(F) and FREE3/ASFMR (color coded) [FIG. 1B]. Individual CPG sites within each region are numbered accordingly. Prominent transcription factor binding sites and methylation sensitive restriction enzyme recognition sites are indicated in capital font, and are listed/identified in Table 3. Numerous HpaII/MspI sites (CCGG) are located throughout the FREE2 A, B and C region.

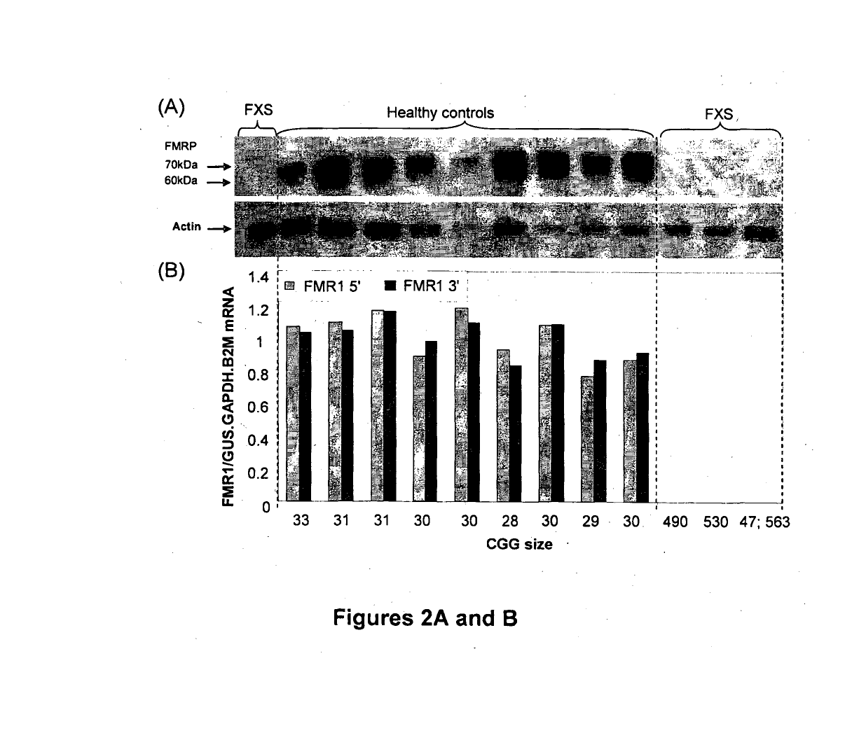

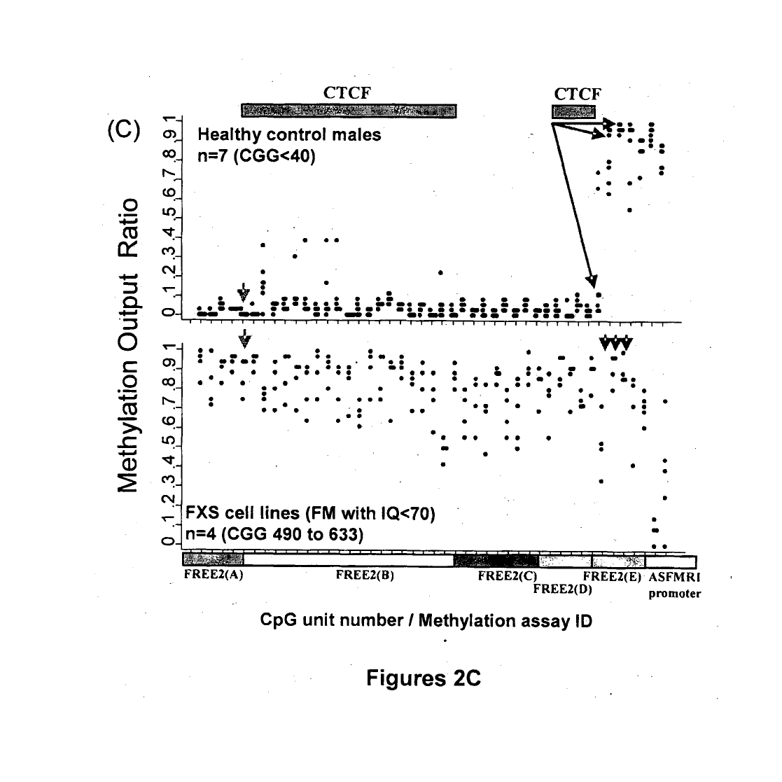

Regions identified as biologically significant showed consistent differences in methylation between healthy controls and FXS samples (FIGS. 2A through C). These include HpaII/MspI sites throughout FREE2 A, B, C, D, E and F regions including but not restricted to the FREE2B CCGG sites located at CpGs 6, 9, 13 and between CpGs 25 and 26; as well as FREE2 (C) CCGG site located at CpG1. These would be sensitive to HpaII methylation specific digestion, which can be followed by PCR or other restriction enzyme based methods to assay differential methylation between healthy controls and FXS samples, and potentially carriers of smaller expansion alleles.

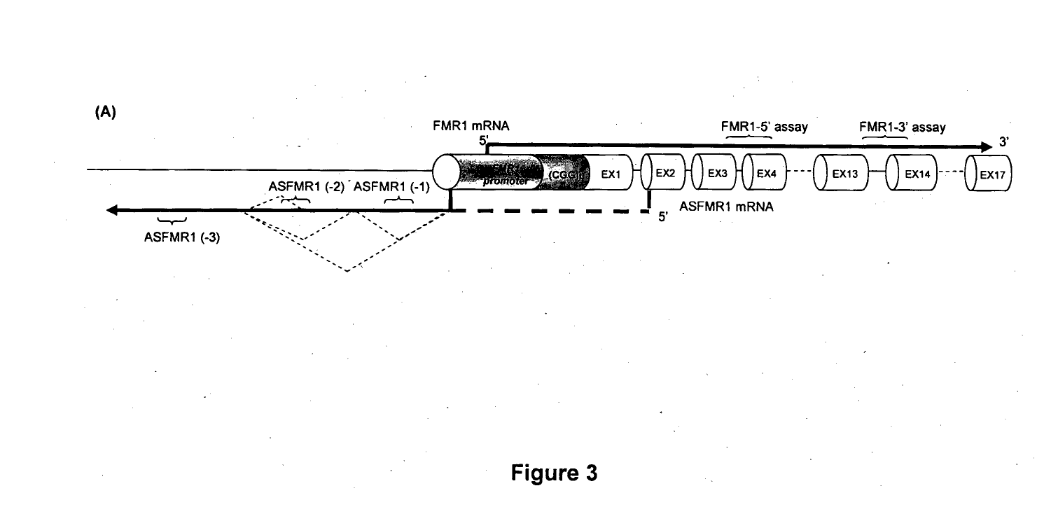

Other regions identified as biologically significant that showed consistent differences in methylation between healthy controls and FXS samples (FIG. 1B and FIG. 2A through C and Table 4) included: (I) GATA-1 site (FREE2B between CpG 15 and 16); (II) HSF2 site (FREE2C between CpG 7, 8 and 9); (III) an SRY site located upstream of FREE3; (IV) a CdxA/TATA box site located at CpG1 of FREE3; (V) an Oct-1 site located between CpG sites 1 and 2 within FREE3. Differential methylation of any of these sites in diseased individuals compared to controls may have an affect of relevant transcription factor binding and/or further epigenetic modification; which would in turn affect transcription of FMR1, ASFMR1 and/or FMR4. Or may result or reflect aberrant non coding RNA expression and/or RNA:DNA interactions or stability of RNA:DNA hybrids (FIGS. 3 and 4).

### Example 2

### Epigenetic Boundary is Located within FREE1, and its Methylation State is Related to Cognitive Impairment

In PM females, significant associations have been identified between cognitive involvement and methylation of the 5′ epigenetic boundary within the FREE1 region. This 5′ boundary is characterized by a downstream CCCTC-binding factor (CTCF) binding site (FIG. 1A). CTCF is a chromatin boundary factor which has been associated with trinucleotide repeat disorders. Methylation shifts from the regions upstream of FREE1 to the FREE1 region in FXS-affected individuals, but not in controls. Naumann et al. (2009) Am J Hum Genet 85(5):606-616 also show that nuclear proteins bind to the 5′ boundary and the FREE 1 region and that the differential methylation of these sequences may affect the level of FMR1 expression in carriers of expanded alleles. Highly significant associations are observed between hypermethylation of FREE1 sequences on this 5′ boundary and the deficit of FMRP in FXS males and females, as well as strong correlations between this hypermethylation and cognitive involvement in PM females as demonstrated by with Wechsler Adult Intelligence Scale (WAIS) IQ tests in PM females: Full Scale IQ (FSIQ) and verbal IQ (VIQ) yielded similar dependencies (R=−0.52, p=0.024, n=18) and Performance IQ (PIQ) yielded R=−0.57, p=0.01, n=18.

### Example 3

### Defining Novel 3′ Epigenetic Boundary and the FMR1 Intron 1 Epigenetic Predictors of FMR1/FMRP and Cognitive Impairment

Data show that FREE2 MALDI-TOF MS methylation analysis of FMR1 intron 1 sequences is superior to methylation-sensitive Southern blot CpG island methylation analysis in blood as a predictor of cognitive impairment, as assessed using WAIS IQ tests, in female carriers of expanded FMR1 alleles. Furthermore, on the basis of methylation of most informative intronic CpG sites, low-functioning FM females can be separated from high-functioning (VIQ<70) FM, PM, and control females with very high specificity and sensitivity approaching 100% [some of these data are now published by the inventor in Godler et al. (2012) Clin. Chem]. Increased methylation of these same intronic sites showed highly significant relationships with decreased WAIS subscores and indexes reflective of impaired verbal abilities. This is particularly important in the context of a significant relationship between deficits in verbal skills and severity of ASD in individuals with large expansions in the FMR1 gene.

Sites designated ACpG 10-12 are identified in the proximal region between FREE(A) and FREE2(B) to be most informative for the severity of cognitive impairment from the comparison of methylation within FREE2(A) Which contains a total of 12 CpG sites (see Godler et al. (2012) supra). ‘These biomarker sites are located at the 5’ end of a large CTCF binding site. Data indicate that there are more of these informative sites well within the FMR1 intron 1. Furthermore, inside the intron there is a novel 3′ epigenetic boundary, with 57 novel biomarker sites, in proximity of the 3′epigenetic boundary and a second smaller CTCF binding site. Data showed the methylation pattern variation was developed graphically in males within the body of the FMR1 gene, 5′ of the CGG expansion between (A) healthy controls CGG<40 (n=20), (B) premutation (n=38), (C) FXS individuals with developmental delay (n=71), (D) premutation/full mutation mosaics (n=24), (E) pure FM methylation mosaics (F) FM unmethylated ‘high functioning’ males (IQ>70)(n=4). Methylation output ratio (Y axis) of 45 CpG units encompassing 69 distinct CpG sites (X axis), were analyzed within the 9.762 kb region 5′ CGG expansion, inclusive of intron I, exon 2 and intron II (sequence numbering from GenBank L29074 L38501) using six SEQUENOM mass spectrometry assays, color coded (see FIG. 1 for sequence location). Red dots were used overlay onto plots (B) to (F) to represent the upper and lower normal methylation range (2 standard deviations from the mean methylation of healthy controls). On the X axis (B)CpG3 and (B)CpG30 units had fragments of the same mass. (B)CpG11 and (B) CpG16 also had fragments of the same mass. SEQUENOM mass. Methylation for these fragments of the same mass; which explains why values are identical for units represented in the same color. Green arrow indicates methylation of a CpG unit 10-12 was noted which was previously described to be significantly associated with the type and severity of cognitive impairment in female carrier of expanded FMR1 alleles. Methylation CpG unit was identified which clearly separates the FM individuals into two distinct groups. Methylation CpG units were identified at the novel 3′ epigenetic boundary.

Additional data determined graphically were of the methylation pattern variation in males within the body of the FMR1 gene, 5′ of the CGG expansion between (A) healthy controls CGG<40 (n=75), (B) premutation (n=141), (C) FM with variable phenotype (n=130), (D) premutation/full mutation mosaics. Methylation output ratio (Y axis) of 45 CpG units encompassing 69 distinct CpG sites (X axis), were analyzed within the 9.762 kb region 5′ CGG expansion, inclusive of intron I, exon 2 and intron II (sequence numbering from GenBank L29074 L38501) using six SEQUENOM mass spectrometry assays, color coded (see FIG. 1 for sequence location). Red dots were used to overlay onto plots (B) to (D) to represent the upper and lower normal methylation range (2 standard deviations from the mean methylation of healthy controls). On the X axis (B)CpG3 and (B)CpG30 units had fragments of the same mass. (B)CpG11 and (B) CpG16 also had fragments of the same mass. SEQUENOM mass spectrometry approach could only provide mean methylation for these fragments of the same mass. Methylation of a CpG unit 10-12 was noted which previously described to be significantly associated with the type and severity of cognitive impairment in female carrier of expanded FMR1 alleles. Methylation CpG units were identified which clearly separate the FM individuals into two distinct groups.

Furthermore, in the FXS group throughout the hypermethylated intron 1, distinct regions are identified which clearly separate the FM males into two groups. These regions correspond to exact locations of CTCF binding sites from ChIP-seq. Interestingly, in the ‘high functioning’ UFM males (IQ>70) that express FMR1 and produce FMRP (40% to 60% of normal levels), the 3′ epigenetic boundary is at the same location as in control males. Together, these data indicate that the 3′ epigenetic boundary, and intron 1 hypomethylation (particularly at CTCF binding sites) is conserved between tissues of healthy controls, PM and ‘high functioning’ UFM individuals, while in FXS affected individuals the 3′ boundary is lost.

Similar to the 5′ boundary located within FREE1, the 3′ epigenetic boundary within FREE2 acts as an insulator, protecting the FMR1 promoter from being hypermethylated in controls, PM carriers, and ‘high functioning’ UFM males. In FM/FXS males with cognitive impairment with no FMR1 expression, the 3′ epigenetic boundary disappears. Interestingly, in the blood the phenomenon of FXS specific methylation does not apply to the CpG sites within the ASFMR1 promoter, as the ASFMR1 promoter is equally hypermethylated in control, PM, low and high functioning FM individuals. However, in FXS lymphoblasts, the ASFMR1 promoter is hypomethylated, while in lymphoblast controls it is hypermethylated, suggesting that differential expression of ASFMR1 contributes to the FXS phenotype in a cell type specific manner.

Mechanistically this is explained by CGG dependent hypermethylation in proximal regions to the boundary (particularly FREE2D) due to chromatin looping, which inhibits CTCF binding responsible for blocking DNA methylation and insulating the gene within the heterochromatin. Loss of CTCF binding at the 3′ boundary is proposed to result in further spreading of hypermethylation into the FMR1 promoter and silencing of FMR1 and/or ASFMR1 expression and FMRP production.

### Example 4

### Differential Methylation at the 3′ Epigenetic Boundary in PM Carriers Compared to Controls

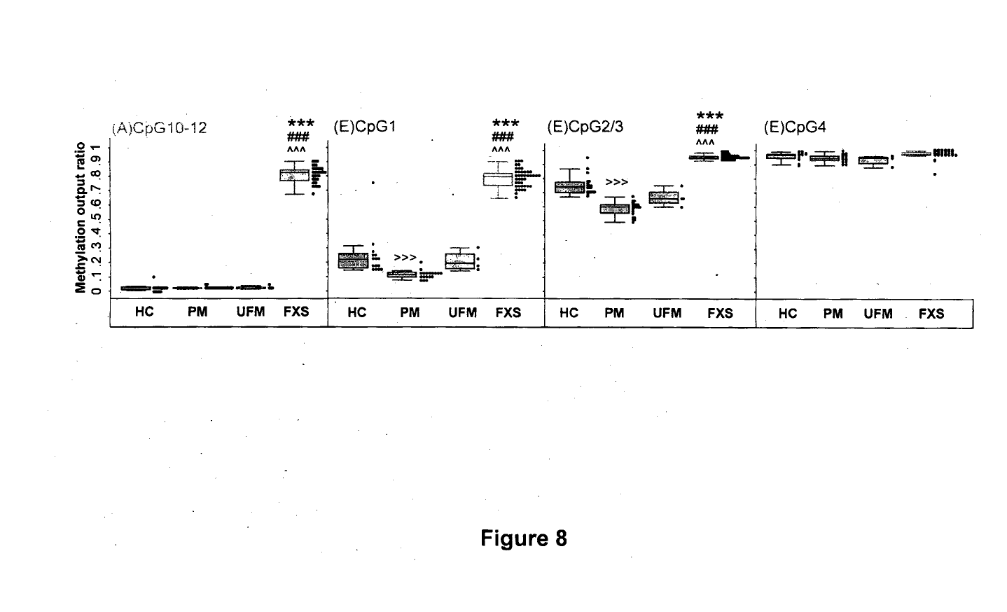

Another observation of interest is that whilst the majority of the intron is hypomethylated in controls and PM males there is a highly significant (p<0.001) decrease in methylation at the 3′ epigenetic boundary (FIG. 8) in the PM compared to all other groups. A comparison of methylation, at the exon 1/intron 1 border and the 3′ epigenetic boundary in control, PM, showed that ‘high functioning’ (IQ>70) FM males were hypomethylated while FM males with cognitive impairment (IQ<70) methylation was significantly increased [P<0.001]. This further supports the proposition of long range epigenetic modification of the 3′ boundary by the CGG repetitive sequence. This PM specific boundary hypomethylation, may be related to levels of FMR1/ASFMR1/FMRP and the learning and behavioral problems including co-morbid autism; and other PM and RNA toxicity related disorders.

### Example 5

### Methylation at Different Ages and Impact on Usefulness in Prognosis

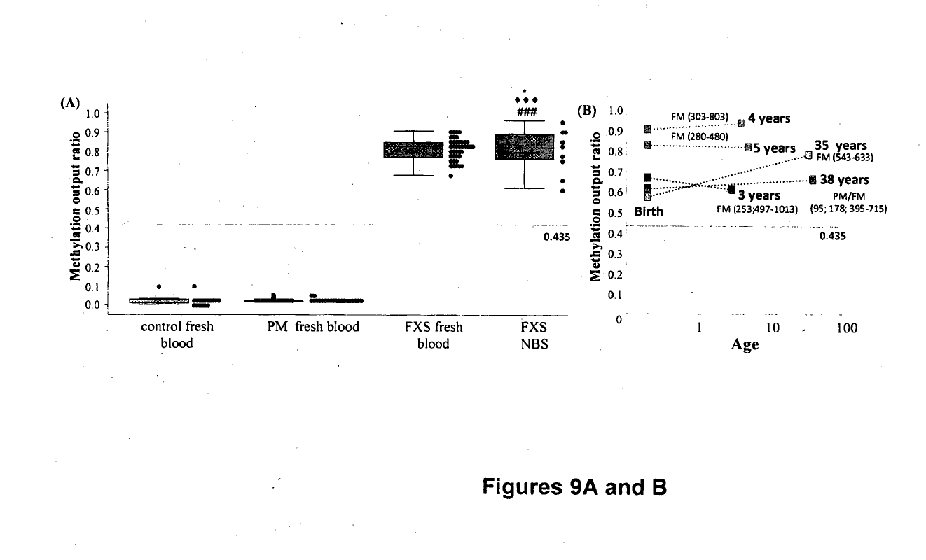

Data show that methylation assessment of the biomarkers, can be done at any time of life and that this will produce similar result as at birth (FIG. 9). More specifically, it was found that for most promising biomarkers to date namely FREE2 CpG 10-12: (i) there was no significant relationship between the age and methylation in 154 females, comprising controls, PM and FM individuals with age range 1.5-67 years; (ii) methylation values for both NBS and fresh blood for 20 FXS males were significantly higher than methylation values for control and PM and healthy controls, and the positive, cognitive impairment threshold of 0.435 determined in our previous epi-genotype-phenotype studies; (iii) in five FXS males the methylation values were almost identical for the repeated methylation measures taken at birth (NBS material) and at 3, 4, 5, 35 and 38 years of age (dried blood spot material) [FIG. 9]. Methylation values for both NBS and fresh blood for these FXS males were significantly higher than methylation values for control and PM, and the positive, cognitive impairment threshold of 0.435.

### Example 6

### Determining the Impact of Technical Variation on Quantitative Analysis of Methylation and Evidence for Disease Specific Methylation within Intron I and Intron II of FMR1

DNA from lymphoblasts of healthy controls with 30 CGG repeats, normal levels of FMR1 mRNA and FMRP, and DNA from lymphoblasts of FXS patient with 530 CGG, silenced FMR1 transcription and absence of FMRP were mixed at ratios of 1:0; 2:1; 1:1; 1:2; 0:1 corresponding to 0, 33.3, 50, 66.6, 100% FXS DNA in the sample. The spiked DNA samples were bisulfite converted in duplicate reactions. Each reaction was amplified with primer sets (forward and reverse primers) as listed by NOs: ID NOs: which corresponded to 3 SEQUENOM mass spectrometry assays (A: FREE2(B); B: FREE2(C); C: FREE3). The spiked DNA samples were analyzed using MALDI-TOF methylation analysis at three sequential regions at the Xq27.3 locus (see FIGS. 1A and B for locations). The methylated vs unmethylated ratios at each analysable CpG unit were expressed as output methylation ratios on Y axis, with FXS DNA input % expressed on the X axis (each point represents mean of duplicate PCRs from a single bisulfite converted DNA mixture). Methylation output ratios for CpG sites within FREE2B and FREE2C amplicons (A and B) were positively correlated with increasing FXS DNA input %; while FREE3 Methylation output ratios were negatively correlated with increasing FXS DNA input % with high Pierson's correlation. This clearly demonstrates that the FREE2 region comprising a large portion of FMR1 intron 1 is hypermethylated in FXS sample while FREE3 region within intron 2 of FMR1 is hypomethylated in the FXS sample. This methylation pattern is reversed in the healthy control sample, and supports the differential methylation patterns within FREE2 and FREE3 related to the disease state as shown in FIGS. 2A through C.

### Example 7

### Evidence for Expression of ASFMR1 in FXS and Disease Specific RNA:DNA Interactions

Standard curve and amplification real-time PCR plots (of assays described in FIG. 3) show that in the FXS cell lines with fully methylated FMR1 promoter and silenced FMR1 and FMRP, ASFMR1 is expressed. RNA was extracted from 3 FXS cell lines whose methylation profiles are presented in FIG. 2; Sample 849 was taken from the male 490 CGG repeat line; Sample 8.62 was taken from the male 530 CGG repeat line; Sample 865 was taken from the female 563 and 47 CGG repeat line. Each RNA sample was split in two, with one half subjected to Rnase A treatment prior to ASFMR1 (−3) relative standard curve analysis. The ASFMR1 (−3) real-time PCR analysis was performed in quadruplicate reactions. The difference in Ct values between Rnase A treated and untreated samples represents the level of ASFMR1 expression.

Standard curve and amplification real-time PCR plots also indicate that in the FXS cell lines, ASFMR1RNA forms RNA:DNA complexes. FXS RNA samples were treated with TURBO Dnase and RQ1 Dnase. These Dnase treatments caused complete loss of real-time-PCR signal for the ASFMR1 (−3) assay. Because Dnase can only degrade RNA molecules if they form complexes with DNA, loss of ASFMR1 after Dnase treatment suggests that ASFMR1RNA forms RNA:DNA complexes in FXS samples, with fully methylated FMR1 promoter and silenced FMR1 expression.

Expression of different FMR1 and ASFMR1 transcripts (detailed in FIG. 3) was detected in RNA samples from lymphoblast lines of 6 male controls, two FXS males (samples 849 and 862) and one FXS female (865) [FIGS. 4A through C]. The control and FXS RNA samples were either treated with TURBO Dnase (A), RQ1 Dnase (B), Rnase A (C), or were untreated. Addition of TURBO Dnase or RQ1 DNAse buffers to RNA samples without Dnase were included as additional controls in (FIGS. 4A through C). The FMR1 and ASFMR1 transcripts were quantified using real-time RT-PCR relative standard curve method, ‘normalized to mRNA levels of three internal control genes, GUS, GAPDH and B2M. FMR15’ and 3′ assays showed no signal for the FXS RNA samples, while similar levels were detected in all control samples (FIGS. 4A, B and C). TURBO and RQ1 DNAse treatment cased ˜50% decrease in the FMR1 levels in most of the control samples; while Rnase A treatment caused complete loss of FMR1 and ASFMR1 signals. While decrease of ASMFR1 (−1), (−2) and (−3) levels was also observed in all control samples caused by TURBO and RQ1 DNAse treatment, in FXS samples (with analogous to control ASFMR1 levels in the untreated samples) TURBO and RQ1 DNAse treatment resulted in complete loss of signal for all three ASFMR1 assays. Because Dnase can only degrade RNA molecules if they form complexes with DNA, This suggests that ASFMR1 RNA forms RNA:DNA complexes more readily in FXS samples than in controls. Increase in RNA:DNA interaction of ASFMR1 in FXS may lead to methylated FMR1 promoter and adjacent regions (FIGS. 1A and B) and silencing FMR1 expression leading to loss of FMRP and the resulting FXS clinical phenotype.

### Example 8

### FMR1 Intron 1 Methylation in Blood Predicts Cognitive Impairment in Female Carriers of Expanded FMR1 Alleles

FMR1 Intron 1 Methylation in Blood is Significantly Correlated with Cognitive Scores

The aim of this Example was to identify those FREE2 CpG sites within intron 1 of the FMR1 gene which can be most effectively used to detect low functioning FM human females amongst control, PM and FM human females. The data on full scale IQ (FIQ), verbal IQ (VIQ) and performance IQ (PIQ), as well as whole blood DNA were available in this cohort, and DNA was re-tested in this study using the Sequenom EpiTYPER system and Southern blot tools to determine methylation output ratio and activation ratio, respectively. For the molecular analyses the cohort was separated into four groups: controls (<40 repeats) (n=21); PM carriers (n=63) with FIQ, VIQ and PIQ greater than 70; high functioning FM carriers with FIQ>70 (n=12), VIQ>70 (n=13) and PIQ>70 (n=11); and low functioning F M human females FIQ<70 (n=10), VIQ<70 (n=9) and PIQ<70 (n=11). An IQ of 70 was considered as a cutoff for mental retardation following the criteria used in the earlier relevant studies (de Vries et al., 1996 supra; Taylor et al., 1994 supra).

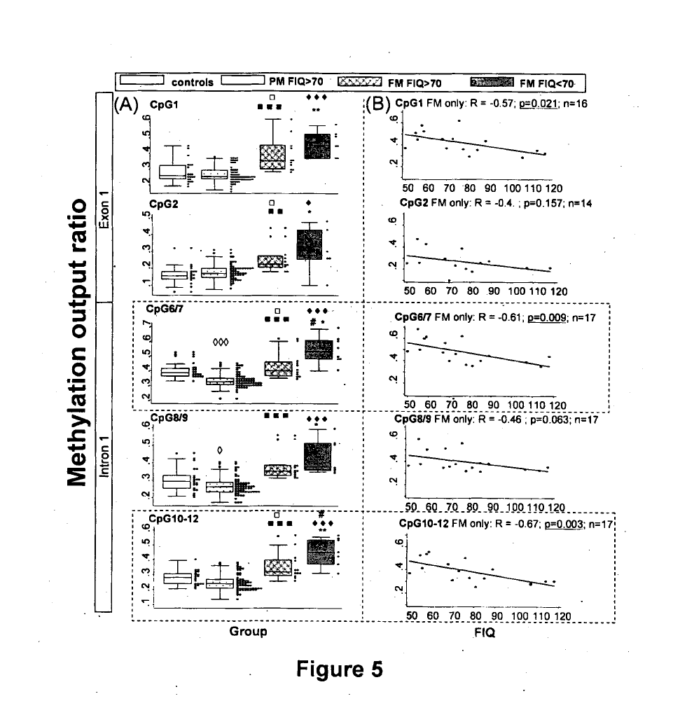

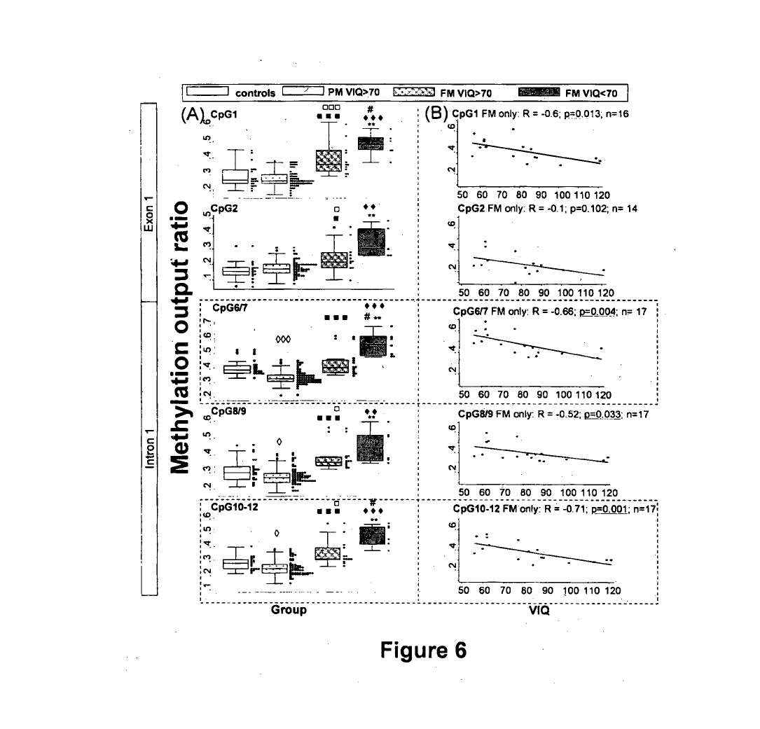

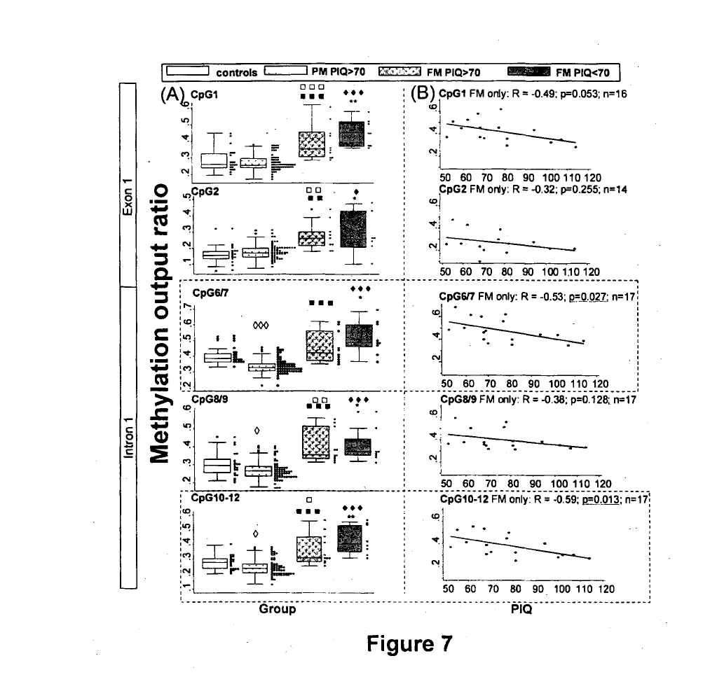

It was found that the median percentage of methylation of FREE2 intronic and exonic units were significantly higher in FM (p<0.01) compared to PM carriers and controls (FIGS. 5, 6 and 7). FREE2 intronic CpG units 6/7 and 10-12 consistently showed significantly elevated median percentage of methylation in FM human females with FIQ and VIQ<70 compared to FM carriers with FIQ and VIQ>70 (FIGS. 6A and 7A). If the FM group was separated based on PIQ>70 (FIG. 7A) the median percentage of methylation was not significantly different in the low functioning compared to the high functioning FM human females. Of the exonic units, only CpG1 showed significantly elevated median percentage of methylation in FM human females with VIQ<70 compared to FM carriers with VIQ>70 (FIG. 6A). These differences could not be accounted for the age of the participants as there was no significant correlation between age and the methylation output ratio for any of the FREE2 units in either control, PM or FM groups.

The sensitivity was further determined, which represents the proportion of the low functioning females (IQ<70) identified as having a positive methylation test result, and specificity, which represented the proportion of borderline to normal IQ (IQ>70) identified as having a negative methylation test result. For the intronic CpG units 6/7 and 10-12 the sensitivity was 100% for detection of low functioning FM carriers (Table 5). The specificity for CpG unit 6/7 was 95%, while for the CpG unit 10-12 it was 94%.

TABLE 5

| | VIQ < 70 | | | VIQ < 70 | |
|---|---|---|---|---|---|
| (A) CpG 6/7 | Present | Absent | (B) CpG 10-12 | Present | Absent |
| Positive | 7 | 5 | Positive | 6 | 6 |
| (>0.47) | | | (>0.37) | | |
| Negative | 0 | 89 | Negative | 0 | 91 |
| (<0.47) | | | (<0.37) | | |
| Total | 7 | 94 | | 6 | 97 |
| Sensitivity | 100% | | | 100% | |
| Specificity | | 95% | | | 94% |

Two outliers from the control cohort for CpG unit 6/7 above the positive threshold of 0.47, and one outlier from the control cohort for CpG unit 10-12 above the positive threshold of 0.37, represented females with normal size CGG alleles, but with clinical history of developmental delay. Two outliers that had methylation output ratio for CpG unit 6/7 above the positive threshold of 0.47 represented FM human females, where methylation output ratios were 0.6 and 0.62 with VIQ values of 95 and 77, respectively. Four other FM outliers that had methylation output ratios for CpG unit 10-12 above the positive threshold of 0.37 had methylation output ratios of 0.39, 0.4, 0.5 and 0.56 with VIQ values of 82, 86, 77 and 95, respectively.

In the FM only cohort, FREE2 methylation showed significant correlation with FIQ, VIQ and to a lesser extent with PIQ (FIGS. 5B, 6B and 7B). Exonic CpG1 showed correlation of borderline significance with FIQ, VIQ, but not PIQ, whereas intronic CpG units 6/7 and 10-12 showed much stronger correlation with all these measures. Intronic CpG unit 8/9 also showed significant correlation with VIQ, but not with FIQ or PIQ. Furthermore, methylation of intronic CpG units were significantly correlated with VIQ indices (Verbal Comprehension and Working Memory) and subtest scores (Vocabulary, Similarities, Information, Comprehension, Arithmetic and Digit span) [Table 6]. Interestingly, even in the high functioning group of FM human females (VIQ>70) the methylation output ratio of FMR1 intron units was related to the impairment of the Arithmetic skills. Furthermore, of all the cognitive sub-scores, Arithmetic which is largely dependent on working memory and attention skills, was also the assessment that showed the strongest correlation with methylation of exon CpG1 and 2 units (p<0.001) [Table 6]. Information and Picture Comprehension were the other subscores that correlated with methylation of exon CpG1 and 2 units, but the relationships were of borderline significance (p<0.05) [Table 6].

TABLE 6

| | Verbal | | | | | | | | | | Working | | | |
|---|---|---|---|---|---|---|---|---|---|---|---|---|---|---|
| | Comprehension | | | | | | | | | | Memory | | | |
| | Index | | Vocabulary | | Similarities | | Information | | Comprehension | | Index | | Arithmetic | |
| Variable | n | P | n | p | n | p | n | p | n | p | N | P | n | p |
| CpG1 | 13 | 0.161 | 14 | 0.124 | 15 | 0.199 | 15 | 0.04 | 15 | 0.077 | 9 | 0.036 | 15 | 0.001 |
| CpG2 | 12 | 0.187 | 13 | 0.154 | 14 | 0.566 | 14 | 0.037 | 14 | 0.147 | 7 | 0.211 | 14 | 0.005 |
| CpG6-7 | 14 | 0.01 | 15 | 0.016 | 16 | 0.06 | 16 | 0.0006 | 16 | 0.039 | 10 | 0.02 | 16 | 0.001 |
| CpG8-9 | 15 | 0.058 | 16 | 0.77 | 17 | 0.2 | 17 | 0.041 | 17 | 0.06 | 10 | 0.1 | 17 | 0.002 |
| CpG10-12 | 14 | 0.015 | 15 | 0.016 | 16 | 0.042 | 16 | 0.002 | 16 | 0.02 | 9 | 0.018 | 16 | <0.0001 |

| | | | | | | | | | | | | | Southern | |
|---|---|---|---|---|---|---|---|---|---|---|---|---|---|---|
| | | | Perceptual | | | | | | | | | | blot | |
| | | | Organization | | Block | | Picture | | Picture | | FMRP % | | Activation | |
| | Digit Span | | Index | | Design | | Completion | | Arrangement | | positive | | ratio | |
| Variable | n | P | n | p | n | p | n | p | n | p | n | p | n | P |
| CpG1 | 14 | 0.163 | 13 | 0.075 | 15 | 0.091 | 15 | 0.042 | 13 | 0.141 | 16 | 0.352 | 16 | 0.091 |
| CpG2 | 13 | 0.181 | 12 | 0.327 | 14 | 0.095 | 14 | 0.261 | 12 | 0.558 | 14 | 0.059 | 14 | 0.019 |
| CpG6-7 | 15 | 0.042 | 14 | 0.056 | 16 | 0.031 | 16 | 0.029 | 14 | 0.079 | 17 | 0.1 | 17 | 0.033 |
| CpG8-9 | 16 | 0.214 | 15 | 0.201 | 17 | 0.22 | 17 | 0.051 | 15 | 0.276 | 17 | 0.067 | 17 | 0.024 |
| CpG10-12 | 15 | 0.125 | 14 | 0.027 | 16 | 0.02 | 16 | 0.019 | 14 | 0.032 | 17 | 0.215 | 17 | 0.069 |

FMRP Immunostaining and Southern Blot FMR1 Activation Ratio in Blood are not Correlated with Cognitive Scores

In the FM cohort, the proportion of FMRP positive cells in blood did not show significant correlation with methylation of any of the FREE2 units in this small sample set. (Table 6). On the other hand, the Southern blot activation ratio showed significant correlations with exonic CpG unit 2 and intronic CpG units 6/7 and 8/9 (Table 6). However, in contrast with methylation output for the intronic units, neither FMRP immunostaining nor Southern blot activation ratio could effectively separate the low functioning individuals based on FIQ, VIQ and PIQ less than 70. Although FMRP immunostaining and Southern blot activation ratio showed highly significant correlation in blood (R=0.8 p>0.0001; n=18), neither measure showed significant correlation with any of the IQ scores or cognitive sub-scores which showed strong correlation with methylation of intronic CpG units 6/7 and 10-12 (Table 6).

It is clear from the results that, of all molecular measures examined, methylation of FMR1 intronic CpG units 6/7 and 10-12 (and, to a lesser extent, unit 8/9), is the most significant predictor of cognitive impairment in FM human females. Methylation of the intronic CpG units also showed correlation with Southern blot FMR1 activation ratio, but correlation with FMRP expression was not significant (p=0.06). Furthermore, neither Southern blot FMR1 activation ratio nor FMRP expression in blood showed significant correlation with any of the cognitive measures in this small sample.

Although Southern blot analysis combined with PCR has been the current ‘gold standard’ in FXS diagnostics, the estimated relationships of these results with cognitive status are inconsistent. Kaufmann et al. (1999) supra reported that the Southern blot activation ratio is significantly associated with the full scale IQ (FIQ) in FM human females. de Vries et al. (1996) supra confirmed these findings, and also reported that PIQ, but not VIQ, was significantly correlated with the activation ratio. While Taylor et al. (1994) supra did not find any correlations between these cognitive measures and activation ratio. The findings of Taylor et al. (1994) supra showed a lack of correlations of Southern blot outcomes with cognitive status in female blood. However in the same samples, there is a highly significant correlation between methylation of intronic units 6/7 and 10-12 assessed using the MALDI-TOF MS and FIQ, VIQ and to a lesser extent PIQ. Since FIQ is the sum of VIQ and PIQ, the significance of the observed relationship between FIQ and intronic methylation can be largely contributed to the high VIQ-methylation correlations.

Furthermore, on the basis of methylation of these highly informative intronic units 6/7 and 10-12 low functioning FM human females can be separated from high functioning FM, PM, and control females with specificity and sensitivity approaching 100%, if the affected status is determined by the VIQ (Wechsler (1997) ed. The Wechsler Adult Intelligence Scale-Third Edition: Administration and Scoring Manual. 3 ed.: Orlando: The Psychological Corporation) score. Therefore, hypermethylation of these intronic units is a powerful blood diagnostic marker of cognitive status in FM human females which, clearly, showed a much higher sensitivity assessed in the small cohort than Southern blot activation ratio or FMRP expression in blood. It is also important to emphasize that the positive methylation thresholds of 0.47 for CpG6/7 and 0.37 for CpG10-12 as determined in this example could be effectively used to separate low functioning from high functioning FM human females. Diagnostic validity of these thresholds importance will be further emphasized if they are confirmed in a larger, independent cohort.

Since the VIQ is merely a combination of scores representing different aspects of verbal skills and memory, consideration was given to the set of IQ subtests of the VIQ in correlation analysis with methylation outputs and FMRP levels in FM human females. It was found that only the following subtest scores included in the VIQ (Arithmetic, Information, Vocabulary, Comprehension, Similarities and Digit Span) were significantly correlated with methylation of intronic units 6/7 and 10-12, in contrast with Southern blot methylation and FMRP expression, which did not show significant correlation with these measures.

Arithmetic skills, which largely rely on the working memory and attention, stood out as the subtest score showing the highest correlations with intronic methylation, and the only clinical measure that showed a highly significance significant relationship with methylation of the exonic units. In some earlier studies Arithmetic skills were found impaired in FM human females even in the absence of other cognitive impairments (Lachiewicz et al. (2006) Am J Med Genet A. 140(7):665-672; Wisniewski et al. (1985) Ann Neruol 18(6):665-669). Consistent with these data, it was found that even in high-functioning FM human females with VIQ>70, the FREE2 analysis can identify individuals with specific impairments in Arithmetic. None of the PCR or MLPA tests previously developed for FMR1 methylation analysis (Hornstra et al. (1993) supra; Boyd et al. (2006) supra; Nygren et al. (2008) supra; Zhou et al. (2006) supra; Dahl et al. (2007) supra; Dahl and Guldberg (2007) supra; Dahl and Guldberg (2007) supra; Weisenberger et al. (2005) supra; Weinhausel and Haas (2001) supra; Coffee et al. (2009) supra; Stoger et al. (1997) supra; Rosales-Reynoso et al. (2007) supra; Zhou et al. (2004) supra) showed a comparable degree of association of both severity of the general cognitive impairment and individual aspects of the impairment with the methylation status as shown for intronic/exonic FREE2 sites in this study. Thus, use of FREE2 methylation analysis in FXS molecular diagnostics minimizes the number of FM human females which are misclassified as not affected based on overall assessment of cognitive ability and/or behavioral deficiency such as IQ, as these females may still have deficiencies in specific cognitive skills, notably working memory and attention. It also appears from the data that methylation of the FREE2 sites does not significantly change with the age within the range of 2-67 years, suggesting that methylation of these sites also has a prognostic value.

The additional advantage of using differential methylation of the FREE2 sequences on the FMR1 exon 1-inton 1 boundary is that this methylation may be stable between different tissues, so that it may affect gene activity through FMR1 splicing equally in blood and brain. This concept, which has been indicated by whole genome methylation studies (Maunakea et al. (2010) Nature 466(7303):253-257; Laurent et al. (2010) Genome Res 20(3):320-331) is also consistent with the results showing the close relationship of the level of hypermethylation of FREE2 sequences with the severity of cognitive impairment. Functional studies of the regulation of FMR1 activity are essential to confirm or refute this hypothesis. Furthermore, results presented in this study for FXS may also highlight the importance of epigenetic modification of intronic-exonic boundary sequences and intragenic methylation in general on clinical outcome in other triplet-repeat nucleotide expansion disorders.

The data show that FREE2 MALDI-TOF MS methylation analysis of FMR1 intron 1 sequences is superior to methylation sensitive Southern blot and FMRP immunostaining in blood as a predictor of cognitive impairment in female carriers of expanded FMR1 alleles. Because previously developed PCR based tests for FMR1 CpG island methylation analysis (Coffee et al. (2009) supra) are only clinically meaningful in males, the demonstrated ability to identify affected females, as well as males with high sensitivity, places MALDI-TOF MS analysis of intron 1 methylation in a unique position as the preferred tool for newborn screening. Considering its high-throughput and specificity for pathogenic FM alleles, and minimal-DNA-requirement, further validation and implementation of this test into diagnostic settings may offer an ethically acceptable, simple, accurate and inexpensive test available for FXS mass population screening as well as targeted screening in both males and females.

### Example 9

### Determination of Informative CpG Biomarker Sites within the FREE Regions for the Severity of Specific Cognitive Impairments/Autistic Behaviours and for Abnormal FMR1/ASFMR1 mRNA and FMRP Levels in FMR1 Expansion Carriers

Patient Cohort:

A total of 409 archival blood DNA samples from carriers of expanded FMR1 alleles and, healthy controls, with parallel ADOS and WISC IQ scores and sub-score data are assessed. These include 227 males (71 FM, 16 PM/FM mosaics, 70 PM males and 70 controls) and 182 females (40 FM, 116 PM and 20 controls).

Cognitive and behavioral assessments are also performed on an additional 120 FMR1 expansion carriers (60 FM and 60 PM) and 30 age matched controls. Blood and saliva are collected from these 150 individuals. In addition, blood and saliva from 40 controls are assessed.

In total for the epi-genotype-phenotype studies, there is access to DNA from 433 FMR1 expansion carriers (171 FM, 246 PM, and 16 PM/FM mosaics) and 166 controls. For the analysis of the relationships between the epi-genotype and FMR1/ASFMR1 mRNA and FMRP levels, RNA and prepare protein lysates from blood samples of the freshly recruited 120 carriers (60 FM, 60 PM) and 70 age matched controls.

Neuropsychological Assessments:

The individuals whose archival data and DNA samples are assessed using the Autism Diagnostic Observation Schedule-Generic (ADOS-G) (Loesch et al. (2007) Neurosci Behav Rev 31(3):315-326) and Wechsler intelligence test appropriate for chronological age: WPPSI-III for ages less than 6, WISC-III for ages between 6 and 16 years and WAIS-III for ages greater than 16 years. The cognitive status of these individuals is described in Loesch et al. (2007) supra, and this information is used for the epi-genotype-phenotype assessments. Additional PM and FM individuals recruited in this study undergo the same clinical assessments.

Blood Processing:

10 to 15 ml of blood is collected in EDTA-treated tubes from freshly recruited FMR1 expansion carriers (60 FM, 60 PM) and 30 age matched controls. DNA for methylation analyses is extracted from 5 ml of whole blood using NucleoSpin (Registered Trade Mark) Tissue genomic DNA extraction kit, as per manufacturer's instructions (MACHEREY-NAGEL GmbH & Co. KG, Düren, Germany). Ten ml of blood is used for peripheral blood mononuclear cell (PBMC) isolation using Ficoll gradient separation. The isolated PBMCs be split into two equal fractions for: (i) RNA extraction (RNeasy kit; Qiagen) for gene expression analyses; (ii) cell lysate for Western blot FMRP analysis.

RNA Extractions and Reverse Transcription Real-Time PCR:

The 7900HT Fast Real Time PCR (Applied Biosystems) is used to quantify FMR1-5′, FMR1-3′, ASFMR1, and three internal control genes using the relative standard curve method. All of the above assays are single-plexed, using PCR conditions described in Godler et al. (2009) BMC Clincial Pathol 9(1):5.

Methylation Analysis of FREE1 and FREE2 Sequences:

Sequenom EpiTYPER system is used to analyze 7 amplicons detailed in FIG. 1A in blood DNA from the total sample of 433 FMR1 expansion carriers (171 FM, 246 PM, and 16 PM/FM mosaics) and 166 controls.

Western Blot FMRP Analysis:

FMRP quantification are performed in PBMCs on 20 μg of total protein lysate, using appropriate FXS negative controls and standard curve samples as previously described (Kaufmann et al. (1999) supra).

Statistical Analysis:

Nonparametric regression is used to determine whether there is a non-linear relationship between each predictor of FREE1 and FREE2 methylation levels in PM and FM individuals, and cognitive/behavioral and molecular outcome measures in the combined sample of 433 expansion carriers; (ii) FMR1/ASFMR1 mRNA and FMRP levels in blood samples of the freshly recruited 120 carriers. A best subset of CpG variables that are independently associated with cognitive measures, FMR1/ASFMR1 expression and FMRP in blood are identified using stepwise forward/backward multiple logistic regression model using the Bayesian Information Criterion (BIC) and validated (Godler et al. (2012) supra). Finally, Anova or nonparametric Kruskal-Wallis rank test are used to compare the differences in the levels of methylation for specific CpG units within FREE1 and FREE2 regions, and the neuropsychological and molecular measures between the PM, FM and age matched control groups.

Power calculation for the methylation of the FREE1 5′ epigenetic boundary and (A)CpG10-12 FREE2 biomarker linear regression (at 5% significance level) with all cognitive/behavioral assessment in PM and PM females, based on the parameters from preliminary results (n=18), yielded power of 0.994 to 1.00 for the sample size proposed for in the current study. Power calculation for the methylation of the 3′ epigenetic boundary (E)CpG2.3 FREE2, using t-test (at 5% level of significance) for testing population means between controls, PM and FM males, based on the parameters from preliminary results (controls n=19; PM n=21; FM n=43), yielded power of 1.00 for the current sample size.

Outcome:

If the site specific methylation that separates FM and PM groups at CTCF binding regions and/or the 3′ epigenetic boundary is significantly correlated with abnormal FMR1/ASFMR1 mRNA and FMRP levels and severity of cognitive impairments/autistic behaviours, it provides key mechanistic evidence for the role of intron 1 methylation in regulation of transcription and translation at the FMR1 locus, with implications for development of novel diagnostic and prognostic markers in carriers of PM and FM alleles.

### Example 10

### Determination in Lymphoblasts of FMR1 Expansion Carriers the Functional Significance of Abnormal Methylation at CTCF Binding Sites on CTCF Binding, Preservation of the Epigenetic Boundaries and Prevention of Methylation Spreading into the FMR1 Promoter, and the Affect of these Events on FMR1/ASFMR1/FMRP Production

Lymphoblast Cell Lines:

There are 83 lymphoblast cell lines from patients recruited for FXS/FMR1 studies. Of these 30 are control cell lines (CGG<40); 40 PM (CGGs 56 to 170); 11 FM/FXS (CGGs 200 to 715); 2 ‘high functioning’ (IQ>70) UFM males (CGGs 230 to 650). 25 of these have been already characterized for FMR1 expression, FMRP, and FREE1/2 methylation.

Determining Inter-Group Ranges:

FMR1/ASFMR1 and FMRP analyses are performed, as well as methylation analysis of 7 amplicons (FIG. 1A) in all 83 cell lines, to determine control, PM and FM/FXS ranges for each molecular measure in lymphoblasts. It is determined if there are differences in CTCF binding at the 5′ and 3′ epigenetic boundaries and proximal regions between the control, PM, UFM and FM/FXS cell lines, and whether this differential binding correlates with changes in ASMFR1/FMR1 mRNA and FMRP. CTCF binding is assessed using ChIP/real-time PCR. Rear-time PCR is performed using primers targeting FREE1, FREE2(B) and FREE2(D) amplicons corresponding to CTCF binding sites listed in ChIP-seq.

Effect of Altered Methylation on CTCF Binding at the 5′ and 3′ Epigenetic Boundaries and Proximal Regions:

(i) De-methylation experiments are performed using 3 and 7 day exposures to 5-azadeoxycytidine on 11 FM/FXS cell lines, which have been found partially re-activates FMR1 expression and increases ASFMR1 mRNA levels in our cell lines. The degree of de-methylation using 7 amplicons described in FIG. 1A, and whether region specific de-methylation is associated with increased binding of CTCF to the 5′ and 3′ epigenetic boundaries and proximal regions in FXS cell lines.

Targeted methylation is performed of single CpG sites using: (i) transfection of 16-bp phosphorothioated oligonucleotide (Proligo, Boulder, Colo.) with 5′-methylcytosines (mC) in CpG dinucleotide; (ii) DNA Methyltransferase coupled to a triple helix forming oligonucleotide in control, PM and UFM cell lines. Using these two targeted approaches CpG sites are in vitro methylated within the 5′ and 3′ intragenic epigenetic boundaries and CTCF binding proximal regions that show highest correlation with cognitive and molecular measures in Example 9. Methylation increase is determined at specific CTCF binding sites affects CTCF binding to these regions using ChIP/real-time PCR.

RNAi-Depletion of CTCF:

Knockdown of CTCF is performed using modified siRNA protocols from Thermo Scientific Dharmacon (ON-TARGETplus siRNA Reagents, Thermo Scientific Dharmacon). The effectiveness of the knockdown is monitored using CTCF real time PCR assay. It is then determine whether CTCF knockdown affects methylation of 5′ and 3′ epigenetic boundaries and proximal regions, as well as FMR1/ASFMR1 mRNA and FMRP levels in 5 control, 5 PM and 5 FM/FXS and 2 UFM cell lines.

Statistical Analysis:

As per Example 9, nonparametric regression is used to determine whether there is a non-linear relationship between each predictor of FREE1 and FREE2 methylation and all the other molecular outcome measures, including CTCF binding at the FMR1 locus in lymphoblasts. Nonparametric Kruskal-Wallis rank test is used to compare the differences in the molecular measures between groups.

Outcome:

If the site specific separation deep within FMR1 intron 1 is related to: (i) differential CTCF binding in FXS affected compared to UFM males, PM, and controls; (ii) FMR1/ASMR1 expression and the severity of the phenotype in FM and PM carriers; it provides an avenue for epigenetic treatments targeted at re-activation of FMR1/ASFMR1 transcription in FXS, or reduction of RNA toxicity observed in PM individuals.

### Example 11

### Determination in Saliva the Most Informative CpG Biomarker Sites within the FREE Regions for the Severity of Specific Cognitive Impairments/Autistic Behaviours in the Recruited FMR1 Expansion Carriers

Patient Cohort:

The freshly recruited 120 carriers (60 FM, 60 PM) and 70 age matched controls (see Example 9) is used for the blood/saliva methylation comparison. Neuropsychological assessments in these individuals are performed as in Example 9.

Laboratory Protocols:

Saliva samples are collected using the Oragene® DNA Self-Collection Kit and isolated as per manufacturer's instructions (DNA Genotek Inc., Ottawa, Canada). Blood is collected, and methylation analyses are performed in blood and saliva DNA as detailed in Example 9.

Statistical Analysis:

The sensitivity is considered to be a measure of the probability of correctly identifying the presence of specific cognitive and behavioral deficits as determined using neuropsychological assessments, and the specificity—a measure of the probability of correctly identifying a person not affected. The individuals would be considered as affected with: (i) ASD if the ADOS-G score is >17; (ii) cognitive impairment if IQ test score is <70; or cognitive indexes or cognitive sub-scores are <7.5. Comparisons between the median methylation for each FREE1 and FREE2 unit for blood and saliva DNA between PM and FM groups and healthy controls will be also conducted using the nonparametric Mann-Whitney two-sample test. Furthermore, nonparametric regression will be used to determine whether there is a non-linear relationship between each predictor of FREE1 and FREE2 methylation levels in PM and FM groups in saliva and blood, and cognitive/behavioral outcome for the subsample of 120 carriers.

Outcome:

If methylation of FREE1 and/or FREE2 biomarker sites in saliva DNA provides equal or higher sensitivity, specificity and correlation with severity of specific cognitive and behavioral deficits; and equal or greater differences between affected carriers and controls, it supports the use of non-invasively obtained saliva DNA based methylation testing of FMR1 expansion carriers. This outcome has clear diagnostic and prognostic implication for early detection of cognitive and behavioral deficits as saliva is more suitable than blood for testing in young developmentally delayed or ASD affected children.

### Example 12

### Determination in Blood Spot Material Taken at Birth of PM and FM Individuals, the CpG Sites with Highest Positive Predictive Values for Abnormal FREE Methylation in Venous Blood or Saliva, and for the Severity of Cognitive and Behavioral Impairments at Greater than 5 Years of Age

Newborn Guthrie Spot Samples:

The newborn blood spots have been collected between 1971 and 2012, by a newborn screening program after all newborn screening has been completed. Newborn blood spots are already available for 26 FM individuals recruited by CIC through FXS cascade testing. To complete the dataset, blood spots of additional 34 FM and 60 PM tested in blood and saliva in Example 9, are retrieved from repositories for methylation testing. The methylation results for these 120 expansion carriers (60 FM, 60 PM) in whole blood and saliva taken at the time of consent (greater than 5 years of age, detailed in Example 9) are compared to their results in newborn blood spots. The blood spot results will be also compared to neuropsychological assessments in these individuals as detailed in Example 9.

Laboratory Protocols:

Dried blood spot punches are processed as previously described (Coffee et al. (2009) supra). The methylation analysis of 7 amplicons of FREE 1 and FREE2 sequences in the newborn blood spots, venous blood and saliva DNA are performed as detailed in Example 9.

Statistical Analysis:

The positive predictive values are determined through a retrospective analysis of 60 FM and 60 PM newborn bloodspots using FREE MALDI-TOF MS. The positive predictive values for FREE methylation analysis are calculated as the probability of methylation of specific CpG sites within FREE1 and/or FREE2 regions to provide a positive test result as determined using: (i) methylation analysis in venous blood and saliva DNA at greater than 5 years of age; (ii) neuropsychological assessments in affected subjects greater than 5 years of age. The positive methylation thresholds for each clinical measure are determined using the receiver operating characteristic (ROC) curve analysis and the ability of the methylation value at each CpG site to classify the affected′ and not affected classes for each clinical measure are determined as described.

Outcome:

If findings for (A)CpG10-12 are confirmed in a larger sample of symptomatic and asymptomatic FM and PM individuals, and if other novel CpG sites are shown to be equal or better predictors at birth for the severity and type of the phenotype later in life, this will support the use of the biomarker tests at birth or early infancy as a potential prognostic markers in FMR1 expansion children too young to undergo formal neuropsychological testing.

Those skilled in the art will appreciate that the disclosure described herein is susceptible to variations and modifications other than those specifically described. It is to be understood that the disclosure contemplates all such variations and modifications. The disclosure also enables all of the steps; features, compositions and compounds referred to or indicated in this specification, individually or collectively, and any and all combinations of any two or more of the steps or features or compositions or compounds.

### BIBLIOGRAPHY

Allingham-Hawkins et al. (1999) Am J Med Genet 83(4):322-325
Anido et al. (2007) I Genet Couns. 16(1):97-104
Bailey et al. (2009) J Pediatr Psychol 34(6):648-661
Bodega et al. (2006) Hum Reprod 21(4):952-957
Bonner and Laskey (1974) Eur. J. Biochem. 46: 83
Boyd et al. (2006) Anal Biochem 354(2):266-273
Boyle and Kaufmann (2010) Am J Med Genet C Semin Med Genet 154C(4):469-476
Chiurazzi et al. (1998) Hum Mol Genet 7(1):109113
Coffee et al. (2009) Am J Hum Genet 85(4):503-514
Coffee (2010) Genet Med 12(7):411-412
Coulam (1982) Fertil Steril 38(6):645-655
Dahl and Guldberg (2007) Clin Chem 53(11):1877-1878
Dalh and Guldberg (2007) Nucleic Acids Res 35(21):e144
Dahl et al. (2007) Clin Chem 53(4):790-793 de Vries et al. (1996) Am J Hum Genet. 58:1025-1032
Devys et al. (1992) Am J Med Genet 83(4):208-216
Fahy et al. (1991) PCR Methods Appl. 1(1):25-33
Filipovic-Sadic et al. (2010) Clin Chem 56:399-408
Fleige and Pfaffl (2006) Mol Aspects Med 27(2-3):126-139
Francis et al. (2000) Mol Diagn 5(3):221-225
Fu et al. (1991) Cell 67(6):1047-1058
Gitan et al. (2002) Genome Res. 12(1):158-164
Godler et al. (2009) BMC Clincial Pathol 9(1):5
Godler et al. (2012) Clin. Chem
Godler et al. (2010) Hum Mol Genet, [Epub ahead of print] doi: 10.1093/hmg/ddq1037
Godler et al. (2010) Genet Med 12(9):595
Godler et al. (2010) Hum Mol Genet 19(8):1618-1632
Greco et al. (2002) Brain 125(Pt 8):1760-1771
Hagerman et al. (1994) Am J Med Genet 51(4):298-308
Hagerman et al. (2001) Neurology 57(1):127-130
Hantash et al. (2010) Genet Med 12(3):162-173
Hornstra et al. (1993) Hum Mol Genet 2(10):1659-1665
Irizarry et al. (2009) Nature Genetics 41(2):178-186
Irwin et al (2000) Cereb Cortex 10(10):1038-1044
Jacquemont et al. (2004) Am J Ment Retard 109(2):154-164
Jacquemont et al. (2005) J Med Genet 42(2):e14
Jin and Warren (2000) Hum. Mol. Genet 9(6):901-908
Jin et al. (2003) Neuron 39(5):739-747
Kaufmann et al. (1999) Am J Med Genet 83(4):286-295
Kemper and Bailey (2009) Acad Pediatr 9(2):114-117
Kenneson et al. (2001) Hum Mol. Genet 10(14):1449-1454
Khalil et al. (2008) PLoS ONE 3(1):e1486
Khaniani et al. (2008) Mol Cytogenet 1(1):5
Lachiewicz et al. (2006) Am J Med Genet A. 140(7):665-672
Ladd et al. (2007) Hum Mol Genet 16(24):3174-3187
Laurent et al. (2010) Genome Res 20(3):320-331
Loesch et al. (2002) Am J Med Genet 107(2):136-142
Loesch et al: (2003)Am J Med Genet A 118A(2):127-134
Loesch et al. (2003) Neuropsychology 7(4):646-657
Loesch et al. (2005) Clin Genet 67(5):412-417
Loesch et al. (2007) J Med Genet 44(3):200-204
Loesch et al. (2007) Neurosci Behav Rev 31(3):315-326
Loesch et al. (2011) Genet Med: [in press]
Lyon et al. (2010) J Mol Diagn 12(4):505-511
Maunakea et al. (2010) Nature 466(7303):253-257
Marmur and Doty (1962) J. Mol. Biol. 5: 109
Mitchell et al. (2005) Clin Genet 67(1):38-46
Naumann et al. (2009) Am J Hum Genet 85(5):606-616
Nolin et al. (2003) Am J Hum Genet 72(2):454-464
Nygren et al. (2005) Nucleic Acids Res. 33(14):e128
Nygren et al. (2008) J Mol Diagn 10(6):496-501
Orr and Zoghbi (2007) Ann Rev Neurosci 30:575-621
Pieretti et al. (1991) Cell 66(4):817-822
Pietrobono et al. (2002) Nucleic Acids Res 30(14):3278-3285
Rein et al. (1998) Nucleic Acids Res. 26:2255
Rosales-Reynoso et al. (2007) Genet Test 11(2):153-159
Stoger et al. (1997) Hum Mol Genet 6(11):1791-1801
Sullivan et al. (2005) Hum Reprod 20(2):402-412
Tassone et al. (1999) Am J Med Genet 84(3):250-261
Tassone et al. (2008)J Mol. Diagn 10:43-49
Taylor et al. (1994) Jama 271 (7):507-514
Terracciano et al. (2005) Am J Med Genet C Semin Med Genet 137C(1):32-37
Tost et al. (2003) Nucleic Acids Res 31(9):e50
Turner et al. (1996) Am J Med Genet 64(1):196-197′
Verkerk et al. (1991) Cell 65(5):905-914
Wechsler (1997) ed. The Wechsler Adult Intelligence Scale-Third Edition: Administration and Scoring Manual. 3 ed.: Orlando: The Psychological Corporation
Weinhausel and Haas (2001) Hum Genet 108(6):450-458
Weisenberger et al. (2005) Nucleic Acids Res 33(21):6823-6836
Willemsen et al. (1995) Lancet 345(8958):1147-1148
Wisniewski et al. (1985) Ann Neruol 18(6):665-669
Wojdacz et al. (2007) Nucleic Acids Res. 35(6):e41
Yegnasubramanian et al. (2006) Nucleic Acids Res. 34(3):e19
Yu et al. (1991) Science 252(5010):1179-1181
Zhou et al. (2004) J Med Genet 41(4):e45
Zhou et al. (2006) Clin Chem 52(8):1492-1500

1. A method for identifying a pathological condition in a human subject, said method comprising screening for a change relative to a control in the extent of epigenetic modification within a region of:
(i) the FMR1 gene selected from the group consisting of:
(a) Fragile X-related Epigenetic Element 3 (I) [FREE3 (I)] comprising the nucleotide sequence set forth in SEQ ID NO: 1 or a homolog thereof or portion or part thereof defined by having at least 80% nucleotide sequence identity to SEQ ID NO: 1 or which hybridizes to SEQ ID NO: 1 or its complementary form under medium stringency conditions;
(b) intron 2, comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions; and
(c) an intron, intron/exon boundary and/or splicing region downstream of intron 2 or a homolog thereof or a portion or fragment thereof;
(d) a site within the FREE2 portion of intron 1 in combination with a full mutation (FM); or
(ii) the FMR genetic locus selected from the group consisting of:
(a) two or more of (1) an intron; (2) an intron/exon boundary; and (3) a splicing region;
(b) an intragenic region in combination with an expansion mutation; and
(c) approximately one 7th (seventh) or greater of an intron including an intron/exon boundary and/or a splicing region; wherein a change in the extent of epigenetic modification relative to a control is indicative of the presence or severity of the pathological condition or propensity to develop same.

2. The method of claim 1 wherein the epigenetic modification is methylation.

3. The method of claim 1 wherein the pathological condition is a neurodevelopmental or neurodegenerative disorder or a nucleotide expansion disorder.

4. The method of claim 3 wherein the pathological condition is selected from the list consisting of Fragile X-associated tremor or ataxia (FXTAS), Fragile XE mental retardation (FRAXE), Friedrich's ataxia (FRDA), a premutation-related disorder including Fragile X-associated primary ovarian insufficiency (FXPOI), Fragile type, folic acid type, rare 12 (FRA12A), autism, mental retardation, Klinefelter's syndrome, RNA toxicity disease, Turner's syndrome, a modified X-chromosome and cognitive impairment.

5. The method of claim 1 wherein the cell is a cultured or uncultured or a sample type including Chorionic Villi Sample (CVS) cell, a lymphoblast cell, a blood cell, a dried adult or newborn blood spot, buccal cell, an amniocyte, an EBV transformed lymphoblast cell line, fibroblast or a cell from a saliva or cheek swab.

6. The method of claim 1 wherein an epigenetic assay is conducted in conjunction with an assay which determines the length of (CGG)n expansion within the FMR genetic locus leading to a (CGG), expansion pathology selected from a Gray Zone (GZ) pathology, a premutation (PM) pathology or a full mutation (FM) pathology.

7. A method for screening for an agent which modulates epigenetic modification of an FMR genetic locus in a mammalian cell including a human cell, said method comprising screening for a change relative to a control in the extent of epigenetic change in:
(i) the FMR1 gene selected from the group consisting of:
(a) Fragile X-related Epigenetic Element 3 [FREE3 (I)] comprising the nucleotide sequence set forth in SEQ ID NO: 1 or a homolog thereof or portions or parts thereof defined by having at least 80% nucleotide sequence identity to SEQ ID NO: 1 or which hybridizes to SEQ ID NO: 1 or its complementary form under medium stringency conditions;
(b) intron 2 comprising the nucleotide sequence set forth in SEQ ID NO:2 or a homolog thereof or portions or parts thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or a portion or fragment thereof;
(d) a site in the FREE2 portion of intron 1 in combination with an FM;
(ii) an FMR genetic locus selected from the group consisting of:
(a) two or more of (1) an intron; (2) an intron/exon boundary; and (3) a splicing region,
(b) an intragenic region in combination with an expansion mutation; and
(c) approximately one seventh or greater of an intron including an intron/exon boundary and/or a splicing region;
in the presence or absence of an agent to be tested, wherein the agent is selected if it induces a change in extent of epigenetic modification.

8. The method of claim 7 wherein the epigenetic modification is methylation.

9.-13. (canceled)

14. A method for identifying in a genome of a mammalian cell including a human cell, a pathological condition associated with methylation or other epigenetic modification within the FMR locus, said method comprising:
(A) extracting genomic DNA from said cell,
(B) subjecting the DNA to an amplification reaction using primers selective of a region of the FMR genetic locus within:
(i) the FMR1 gene selected from the group consisting of:
(a) Fragile X-related Epigenetic Element 3 [FREE3 (I)] comprising the nucleotide sequence set forth in SEQ ID NO: 1 or a homolog thereof or portions or parts thereof defined by having at least 80% nucleotide sequence identity to SEQ ID NO: 1 or which hybridizes to SEQ ID NO: 1 or its complementary form under medium stringency conditions;
(b) intron 2 comprising the nucleotide sequence set forth in SEQ ID NO:2 or a homolog thereof or portions or parts thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or a portion or fragment thereof;
(d) a site in the FREE2 portion of intron 1; or
(ii) the FMR genetic locus selected from the group consisting of:
(a) two or more of (1) an intron; (2) an intron/exon boundary; (3) a splicing region; (4) an intragenic region in combination with an expansion mutation; and (5) approximately one seventh or greater of an intron including an intron/exon boundary and/or a splicing region; and
(C) subjecting the amplified and/or enzyme digested DNA to a methylation or other epigenetic assay to determine the extent of methylation or other epigenetic modification of the DNA,
wherein a change in extent of methylation or other epigenetic modification relative to a control is indicative of the presence of the pathological condition or propensity to develop same.

15. The method of claim 14 wherein the pathological condition is a neurodevelopmental or neurodegenerative condition or a nucleotide expansion disorder.

16. The method of claim 15 wherein the pathological condition is selected from Fragile X-associated tremor or ataxia (FXTAS), Fragile XE mental retardation (FRAXE), Friedrich's ataxia (FRDA), Fragile type, folic acid type, rare 12 (FRA12A), autism, mental retardation, Klinefelter's syndrome, RNA toxicity disease, Turner's syndrome, a modified X-chromosome and cognitive impairment.

17. The method of claim 14 wherein the cell is a cultured or uncultured or a sample type including Chorionic Villi Sample (CVS) cell, a lymphoblast cell, a blood cell, a dried adult or newborn blood spot, buccal cell, an amniocyte, an EBV transformed lymphoblast cell line, fibroblast or a cell from a saliva or cheek swab.

18. The method of claim 14, wherein the methylation or other epigenetic assay is conducted in conjunction with an assay which determines the length of a nucleotide expansion leading to an expansion pathology.

19. (canceled)

20. A kit for the use in a method of claim 1 comprising primers which amplify a region with the FMR genetic locus, said region in:
(i) the FMR1 gene selected from the group consisting of:
(a) Fragile X-related Epigenetic Element 3 [FREE3 (I)] comprising the nucleotide sequence set forth in SEQ ID NO: 1 or a homolog thereof or portions or parts thereof defined by having at least 80% nucleotide sequence identity to SEQ ID NO: 1 or which hybridizes to SEQ ID NO: 1 or its complementary form under medium stringency conditions;
(b) intron 2 comprising the nucleotide sequence set forth in SEQ ID NO: 2 or a homolog thereof or portions or parts thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or a portion or fragment thereof;
(d) a site in the FREE2 portion of intron 1 in combination with an FM;
(ii) an FMR genetic locus selected from two or more of (a) an intron; (b) an intron exon boundary; (c) a splicing region (d) an intragenic region in combination with an expansion mutation; and (e) approximately one seventh or greater of an intron including an intron exon boundary and/or a splicing region.

21. The kit of claim 20 wherein the primers are selected from the group list consisting of SEQ ID NOs: 6 through 11.

22. A computer program product for assessing progression of a pathological condition associated with the FMR locus in a subject, the product comprising:
(1) assigning a value to one or more of:
(a) change in of methylation or other epigenetic modification relative to a control in FREE3 (I) of FMR1 comprising the nucleotide sequence set forth in SEQ ID NO: 1 or a homolog thereof or portion or part thereof defined by having at least 80% nucleotide sequence identity to SEQ ID NO: 1 or which hybridizes to SEQ ID NO: 1 or its complementary form under medium stringency conditions;
(b) change of methylation or other epigenetic modification relative to a control in intron 2 of FMR1 comprising the nucleotide sequence set forth in SEQ ID NO: 2 or a homolog thereof or portion or part thereof defined by having at least 80% nucleotide sequence identity to SEQ ID NO: 2 or which hybridizes to SEQ ID NO: 2 or its complementary form under medium stringency conditions;
(c) change of methylation in an intron, intron/exon boundary and/or splicing region downstream of intron 2 of FMR1 or a homolog thereof or a portion or fragment thereof;
(d) two or more of (i) an intron; (ii) an intron/exon boundary; and (iii) a splicing region within the FMR genetic locus;
(e) approximately one 7th (seventh) or greater of an intron, including an intron/exon boundary and/or a splicing region within the FMR genetic locus;
(f) length of (CGG)n expansion within the FMR genetic locus when considered in combination with (a) and/or (b);
(g) hypermethylation at one or more sites in a FREE2 portion on intron 1 of the FMR1 gene in combination with an FM;
(h) general phenotype or clinical manifestations in subjects with a neurodevelopmental or neurodegenerative condition;
(i) behavioral assessment criteria associated with normal subjects, PM subjects, GZ subjects and FM subjects;
(j) cognitive ability and/or behavioral deficiency;
(k) extent of transcription of genes within the FMR locus with the proviso that if any one of (d) through (f) is determined then one or more of (a) through (c) and/or (g) is also determined;
(2) a conversion system to convert the value to a code; and
(3) a computer readable medium to store the code and compare the code to a knowledge database to determine whether the code corresponds to a pathological condition.

23. A computer for assessing an association between extent of methylation or other epigenetic modification within the FMR locus, the FMR locus and progression of a disease condition wherein the computer comprises:
(1) assigning index values to one or more of:
(a) change in of methylation or other epigenetic modification relative to a control at sites within FREE3 (I) comprising the nucleotide sequence set forth in SEQ ID NO: 1 or a homolog thereof or portion or part thereof defined by having at least 80% nucleotide sequence identity to SEQ ID NO: 1 or which hybridizes to SEQ ID NO: 1 or its complementary form under medium stringency conditions;
(b) change of methylation or other epigenetic modification relative to a control at sites within intron 2 of the FMR1 gene comprising the nucleotide sequence set forth in SEQ ID NO:2 or a homolog thereof or portion or part thereof defined by having at least 80% nucleotide sequence identity to SEQ ID NO:2 or which hybridizes to SEQ ID NO:2 or its complementary form under medium stringency conditions;
(c) change of methylation in an intron, intron/exon boundary and/or splicing region downstream of intron 2 of FMR1 or a homolog thereof or a portion or fragment thereof;
(d) two or more (i) an intron; (ii) an intron/exon boundary; (iii) a splicing region within the FMR locus;
(e) approximately one seventh or greater of an intron including an intron/exon boundary and/or a splicing region within the FMR locus;
(f) length of (CGG)n expansion within the FMR genetic locus when considered in combination with (a) and/or (b);
(g) general phenotype or clinical manifestations in subjects with a neurodevelopmental or neurodegenerative condition;
(h) behavioral assessment criteria associated with normal subjects, PM subjects, GZ subjects and FM subjects;
(i) cognitive ability and/or behavioral deficiency;
(j) change in methylation or other epigenetic modification at a site in the FREE2 portion of intron 1 of the FMR1 gene;
(k) extent of transcription of genes within the FMR locus with the proviso that if any one of (d) through (i) is determined then one or more of (a) through (c) is also determined;
(2) a conversion system to convert index value(s) to a code; and
(3) a computer readable medium to store the code and compare code to a knowledge database to determine whether the code corresponds to a pathological condition.

24. A method of identifying epigenetic profile in a population of subjects indicative of a pathological condition associated with the FMR locus, said method comprising screening for a change relative to a control in a statistically significant number of subjects the extent of methylation or other epigenetic modification within:
(i) the FMR1 gene selected from the group consisting of:
(a) Fragile X-related Epigenetic Element 3 [FREE3 (I)] comprising the nucleotide sequence set forth in SEQ ID NO: 1 or a homolog thereof or portions or parts thereof defined by having at least 80% nucleotide sequence identity to SEQ ID NO: 1 or which hybridizes to SEQ ID NO: 1 or its complementary form under medium stringency conditions;
(b) intron 2 comprising the nucleotide sequence set forth in SEQ ID NO:2 or a homolog thereof or portions or parts thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or a portion or fragment thereof; and
(d) a site in the FREE2 portion of intron 1 in combination with an FM;
(ii) an FMR genetic locus selected from the group consisting of two or more of (a) an intron; (b) an intron/exon boundary; (c) a splicing region; (d) an intragenic region in combination with an expansion mutation; and (e) approximately one 7th (seventh) or greater of an intron including an intron/exon boundary and/or a splicing region;
wherein a change in extent of methylation or other epigenetic modification is indicative of the presence of the pathological condition or a propensity to develop same in the population.

25. A method of allowing a user to determine the status, prognosis and/or treatment response of a subject with respect to an FMR locus-associated pathology, the method including:
(1) receiving data in the form of extent of methylation or other epigenetic modification at a site in:
(i) the FM 1 gene selected from the group consisting of:
(a) Fragile X-related Epigenetic Element 3 [FREE3 (I)] comprising the nucleotide sequence set forth in SEQ ID NO: 1 or a homolog thereof or portions or parts thereof defined by having at least 80% nucleotide sequence identity to SEQ ID NO: 1 or which hybridizes to SEQ ID NO: 1 or its complementary form under medium stringency conditions;
(b) intron 2 comprising the nucleotide sequence set forth in SEQ ID NO:2 or a homolog thereof or portions or parts thereof defined by having at least 80% nucleotide sequence identity to SEQ ID NO:2 or which hybridizes to SEQ ID NO:2 or its complementary form under medium stringency conditions;
(c) an intron, intron/exon boundary and/or splicing region downstream of intron 2 or a homolog thereof or a portion or fragment thereof;
(d) a site in the FREE2 portion of intron 1 in combination with an FM;
(ii) the FMR genetic locus selected from: (a) two or more of (a) an intron; (b) an intron/exon boundary; (c) a splicing region; (d) an intragenic region in combination with an expansion mutation; and (e) approximately one seventh or greater of an intron including an intron/exon boundary and/or a splicing region; wherein the extent of methylation or epigenetic modification provides a correlation to the presence, state, classification or progression of the pathology;
(2) transferring the data from the user via a communications network;
(3) processing the subject data via multivariate or univariate analysis to provide a disease index value;
(4) determining the status of the subject in accordance with the results of the disease index value in comparison with predetermined values; and
(5) transferring an indication of the status of the subject to the user via the communications network.
